(19) United States
(12) Patent Application Publication
(10) Pub. No.: US 2026/0185108 A1
(43) Pub. Date: Jul. 2, 2026

(54) GENETIC ENHANCEMENT OF EXOSOME PRODUCTION

(71) Applicant: The Johns Hopkins University, Baltimore, MD (US)

(72) Inventors: Stephen J. Gould, Baltimore, MD (US); Shang-Jui Tsai, Baltimore, MD (US); Yiwei Ai, Baltimore, MD (US); Chenxu Guo, Baltimore, MD (US)

(21) Appl. No.: 19/130,091

(22) PCT Filed: Mar. 16, 2023
(86) PCT No.: PCT/US2023/015417

§ 371 Date: May 14, 2025

Related U.S. Application Data
(60) Provisional application No. 63/427,013, filed on Nov. 21, 2022.

(51) Int. Cl. C12N 15/67; C07K 14/005; C12N 9/06; C12N 15/85

(52) U.S. Cl. CPC C12N 15/67; C07K 14/005; C12N 9/003; C12N 15/85; C07K 2319/01; C12N 2720/12022; C12N 2740/13022; C12N 2740/16122; C12N 2760/16022; C12N 2760/20222; C12N 2770/20022; C12N 2830/48; C12Y 105/01003

(57) ABSTRACT

Levels of expression of antibiotic resistance genes are increased up to six-fold by inserting a proteasome-targeting tag into transgenes expressed in eukaryotic cells. Various selectable marker proteins are combined with different destabilization domains, leading to up to 70% increase in transgene expression. The increase in expression varies highly depending on the engineered construct and the lines cells used. Increase in expression drives exosome loading of cargo proteins in some aspects. By increasing expression and by editing trafficking signals of cargo proteins, proteins that normally locate to the ER can be trafficked to exosomes. This disclosure discloses efficient exosome delivery of a wide variety of engineered proteins, including modified antigen proteins of SARS-CoV-2 and influenza, and other proteins such as a modified alpha galactosidase A, an extracellular domain of vascular endothelial growth factor fused to a constant region of a human immunoglobulin heavy chain, and modified trastuzumab heavy and light chains.

### CROSS-REFERENCE TO RELATED APPLICATIONS

This application claims benefit of priority under 35 U.S.C. § 119(e) of U.S. Provisional Application No. 63/427,013 filed Nov. 21, 2022. The disclosure of the prior application is considered part of and is herein incorporated by reference in the disclosure of this application in its entirety.

### INCORPORATION OF SEQUENCE LISTING

The material in the accompanying sequence listing is hereby incorporated by reference into this application. The accompanying sequence listing xml file, name JHU4440-2 Sequence Listing ST26.xml, was created on Mar. 16, 2023 and is 14 kb.

### FIELD OF THE INVENTION

The invention relates generally to compositions and methods for enhancement of transgene expression in cell lines and more specifically to compositions and methods for increasing protein expression, exosome targeting of cargo proteins, production and purification of engineered exosomes and increasing the yield of exosomes.

### BACKGROUND INFORMATION

In the four decades since the invention of mammalian cell transgenesis, biomedical research has become highly dependent on the creation, analysis, and use of transgenic cell lines. During this time, numerous improvements have been made to nearly every aspect of vector design, including choice of transcriptional control regions, introns, polyadenylation (pAn) sites, mRNA export sequences, translation initiation-promoting sequences, codon utilization, and mode of transgene delivery. As a result, there is now a wealth of information on how to create transgenic mammalian cells, and especially for the production of recombinant proteins of interest.

The choice of antibiotic resistance (AR) gene can have a major impact on transgene expression. Each combination of antibiotic and AR gene/protein establishes a unique threshold of transgene expression below which no cell can survive, suggesting an inverse relationship between (i) the activity and/or stability of each dominant selectable marker protein, and (ii) the average level of transgene expression across a population of antibiotic-resistant cell clones. This hypothesis is relevant to mammalian cell transgenesis in general, but there are several reasons to believe it may be particularly relevant for the production of recombinantly engineered exosomes (REEs).

Exosomes are small secreted organelles (about 30-200 nm diameter) that have the same topology as the cell, are highly enriched in a subset of proteins, lipids, and RNAs, and can transmit signals and molecules through an intercellular vesicle trafficking pathway. Furthermore, exosomes appear to be generated by a stochastic process that operates across a spectrum of plasma and endosome membranes, leading to pronounced compositional heterogeneity of individual exosomes. As a result, failure to maintain high transgene expression in nearly 100% of the cells during the production of exosomes will result in the release of large quantities of unmodified exosomes (UMEs). UMEs are difficult if not impossible to separate from REEs, complicating the production and analysis of REEs. In this context, the field of exosome engineering is likely to benefit particularly from the invention of new AR genes that allow for the rapid selection of polyclonal cell lines in which all surviving cells express high levels of linked proteins of interest.

To create more restrictive AR genes, they were tagged with destabilization domains, or degrons, which target proteins to the proteasome. Degrons from the estrogen receptor (ER50) and E. coli DHFR (ecDHFR) are particularly intriguing, as they can be stabilized in a dose-dependent manner by small molecules (4-hydroxytamoxifen in the case of ER50 and trimethoprim in the case of ecDHFR). Through extensive experimentation, it was found that degron-tagging the five commonly used AR proteins with the ER50 or ecDHFR degrons led to higher levels of linked transgene expression. Improved versions of all five AR genes/proteins were created, culminating in the creation of ER50BleoR, which selects for the highest transgene expression of any selectable marker, and also drives the budding of a recombinant exosomal cargo protein.

Exosomes are small secreted vesicles of ˜30-150 nm diameter that are produced by all human cells, are abundant in all biofluids, are enriched in exosome marker proteins, and can functionally deliver proteins and RNAs to and into cells. These properties make them an attractive vehicle for delivering vaccine antigens and therapeutic proteins, RNAs, and drugs into patients. This idea is also supported by the fact that exosomes are the only bionormal nanovesicle, are normally exchanged in large quantities during breast feeding, sex, and other human behaviors, and are delivered between individuals in large quantities by tissue transplantation, blood transfusion and biofluid injection, all of which have been performed for decades without any evidence of exosome-associated adverse effects. Moreover, toxicology studies have failed to identify any adverse effects from exosome injections, even when the exosomes were of xenogeneic origin (i.e., human exosomes into mice) delivered at doses of 10¹⁰ per injection or more.

### SUMMARY OF THE INVENTION

The present invention is based on the finding that increased transgene expression can be induced by including a degron domain upstream of a selectable marker and downstream of an exosome cargo protein, by expression of the selectable marker downstream of glutamine synthetase, or use of certain homologs of known selectable marker genes that select for particularly high levels of linked transgene expression.

In some embodiments, the disclosure provided herein relates to an isolated polynucleotide sequence encoding a polypeptide including a selectable marker (SM) protein, a degron domain (DD) and an exosome cargo (EC) protein in operable linkage. In certain aspects, the polynucleotide encodes a fusion protein. Another aspect of the disclosure is directed to an isolated polynucleotide, including from a 5′ end to a 3′ end, an EC protein, a DD, and an SM protein. In further aspects, the isolated polynucleotide also includes a first linker between the EC and the DD, and a second linker between the DD and the SM. Additionally, in some aspects, the second linker domain is cleavable or self-cleavable. In various aspects, the SM protein is zeocin resistance protein (BleoR), blasticidin resistance protein (BsdR), G418 resistance protein (NeoR), puromycin resistance protein (PuroR), hygromycin resistance protein (HygR), or a combination thereof. In some aspects, the SM protein is BleoR. In further aspects, the SM protein is PuroR. In additional aspects, the DD is ER50 derived from a human estrogen receptor, or ecDHFR derived from E. coli DHFR. In various aspects, the second linker is a self-cleavable viral 2a peptide. Furthermore, in various aspects described herein, one or more of the coding sequences are operably linked to a regulatory control element. According to some aspects of the disclosure provided herein, the regulatory control element includes a CMV promoter. In various aspects, expression of one or more of the coding sequences in a cell increases the amount of exosomes produced by the cell. In some aspects, expression of one or more of the coding sequences in a cell increases the amount of EC protein within exosomes produced by the cell. In further aspects, the amount of exosomes produced by the cell increases by about at least 500% as compared to a cell that does not include the isolated polynucleotide. In additional aspects, the amount of EC protein within the exosomes is at least about 20-fold higher than an amount of EC protein in exosomes produced by a cell including the isolated polynucleotide lacking a sequence encoding a DD.

In certain aspects of the present disclosure, the disclosure relates to an isolated polynucleotide including a sequence encoding a modified antigen, wherein the sequence encoding the modified antigen includes the isolated polynucleotide. In some aspects, the methods described herein relate to an isolated cell including the isolated polynucleotide.

In some embodiments, the disclosure provided herein relates to a method of producing exosomes, including introducing an isolated polynucleotide into a cell in a first culture media; b) contacting the cells of a) with an antibiotic in a second culture media including said antibiotic, thereby selecting antibiotic resistant cells; c) optionally contacting the cells of a) with culture media that does not include a compound for cell growth; c) expanding the antibiotic resistant cells of b) in a third culture media; d) culturing the expanded antibiotic resistant cells of c) in a fourth culture media; and e) harvesting exosomes from the fourth culture media, thereby producing exosomes. In some aspects of the method described herein, the antibiotic is zeocin. In further aspects of the method described herein, the antibiotic is puromycin. In additional aspects of the methods described herein, the compound essential for cell growth is glutamine.

In some aspects, the disclosure provided herein relates to a pharmaceutical composition including an exosome produced by the methods described herein.

In some embodiments, the methods described herein relate to a method for producing an extracellular vesicle (EV) in a culture media including: (i) inserting an isolated polynucleotide encoding a coding region for an exosome cargo protein (EC) into an expression vector configured to drive recombinant EC expression; (ii) transfecting the expression vector into a cell suitable for producing EVs, thereby generating a transgenic cell; (iii) contacting the transgenic cells with an antibiotic, thereby producing a transgenic cell that expresses a high level of the recombinant EC; (iv) expanding the cell of (iii) in culture media to produce a conditioned culture media; and (iv) collecting EVs from the conditioned culture media. According to some aspects of this disclosure, the coding region includes from a 5′ to a 3′ end: a) a first inverted tandem repeat (ITR-1) flanking b) a region including: a promoter, an exosome cargo protein (EC), a linker peptide (LP), an antibiotic resistance protein (AR), a woodchuck hepatitis virus post-transcriptional regulatory element (WPRE) and a polyadenylation site (pAn); c) a second ITR (ITR-r); d) a Rous sarcoma virus long-terminal repeat (RSV); e) a Sleeping Beauty transposase SB100x; and f) a polyadenylation site (pAn). In some aspects, the AR is selected from the group consisting of zeocin resistance protein (BleoR), blasticidin resistance protein (BsdR), G418 resistance protein (NeoR), puromycin resistance protein (PuroR), hygromycin resistance protein (HygR), and a combination thereof. In other aspects, the AR is linked to a degron domain (DD). In additional aspects, the DD is ER50 derived from the human estrogen receptor, or ecDHFR derived from E. coli DHFR. In some aspects, the EC is CD63/Y235A. In some aspects, high-level expression of CD63/Y235A leads to about 5-fold increase in EV production yield.

Some embodiments provided herein relate to a method of producing an extracellular vesicle (EV) in a culture media including: (i) inserting an isolated polynucleotide encoding a coding region for an exosome cargo protein (EC) into an expression vector configured to drive recombinant EC expression; (ii) transfecting the expression vector into a cell line suitable for producing EVs, thereby generating a transgenic cell; (iii) contacting the transgenic cell with an antibiotic, thereby producing a transgenic cell that expresses a high level of the recombinant EC; (iv) contacting the transgenic cell with a culture media that does not include an essential compound for cell growth; (v) expanding the cell of (iv) in culture media to produce a conditioned culture media; and (vi) collecting EVs from the conditioned culture media. In some aspects of the methods provided herein, the coding region includes from a 5′ to a 3′ end: a) a first inverted tandem repeat (ITR-1) flanking b) a region including: a selectable marker system (SMS), a promoter, an exosome cargo protein (EC), a woodchuck hepatitis virus post-transcriptional regulatory element (WPRE) and a polyadenylation site (pAn); c) a second ITR (ITR-r); d) a Rous sarcoma virus long-terminal repeat (RSV); e) a Sleeping Beauty transposase SB100x; and f) a polyadenylation site (pAn). In some aspects of the methods provided herein, the SMS encodes a polypeptide including a glutamine synthase (GS) protein, a porcine teschovirus 2a peptide linker and an antibiotic resistance (AR) protein. In additional aspects, the SMS further includes a promoter. In various aspects, the EC includes a modified antigen. In some aspects of the methods described herein, the EVs are exosomes or microvesicles. In various aspects of the methods provided herein, the cell suitable for producing EVs is a 293F-derived cell. In further aspects of the methods described herein, about 50% of the EVs include a modified SARS-CoV-2 Spike protein.

In additional aspects of the methods described herein, the disclosure provided herein relates to a pharmaceutical composition including an exosome produced by the methods described herein.

In various embodiments of the disclosure provided herein, the disclosure relates to an expression vector wherein the coding region includes from a 5′ to a 3′ end: a) a first inverted tandem repeat (ITR-1) flanking b) a region including: a promoter, an exosome cargo protein (EC), a linker peptide (LP), an antibiotic resistance protein (AR), a woodchuck hepatitis virus post-transcriptional regulatory element (WPRE) and a polyadenylation site (pAn); c) a second ITR (ITR-r) d) a Rous sarcoma virus long-terminal repeat (RSV); e) a Sleeping Beauty transposase SB100x; and f) a polyadenylation site (pAn). In some aspects, the EC is CD63/Y235A. In further aspects, the EC includes a modified antigen. In additional aspects, the AR is selected from the group consisting of zeocin resistance protein (BleoR), blasticidin resistance protein (BsdR), G418 resistance protein (NeoR), puromycin resistance protein (PuroR), hygromycin resistance protein (HygR), and a combination thereof. In other aspects, the AR is linked to a degron domain (DD). In additional aspects, wherein the DD is ER50 derived from a human estrogen receptor, or ecDHFR derived from E. coli DHFR.

In some embodiments of the disclosure provided herein, the invention relates to an isolated polynucleotide sequence encoding a modified antigen, wherein the modified antigen includes a modified lysosome sorting peptide, a linker protein, and a modified COPI-binding CTT peptide. In some aspects, the modified lysosome sorting peptide disrupts delivery of the modified antigen to lysosomes and increases delivery of the modified antigen to a plasma membrane. Additionally, in some aspects, the modified antigen has increased immunogenicity compared to a naturally occurring antigen. In further aspects, the linker protein is the porcine teschovirus 2a peptide. In additional aspects, the modified antigen is a SARS-CoV-2 Spike protein.

In various aspects of the disclosure provided herein, the disclosure relates to a modified SARS-CoV-2 Spike protein wherein the modified lysosome sorting peptide includes a diproline substitution. In some aspects, the diproline substitution includes 986KV987-to-986PP987. In further aspects, the diproline substitution increases cell surface Spike protein expression by about 500%. In some aspects, the modified COPI-binding CTT peptide includes a diacidic ER export signal (ERES). In additional aspects, the SARS-CoV-2 Spike protein is a SARS-CoV-2 Spike protein from a SARS-CoV-2 delta (S^{delta}) virus. Various aspects of the disclosure provided herein relate to a modified SARS-CoV-2 Spike protein, wherein the modified SARS-CoV-2 Spike protein includes a mutation selected from the group consisting of T19R, G142D, D157-158, L452R, T478K, D614G, P681R, and D950N. Additionally provided are aspects wherein the modified SARS-CoV-2 Spike protein further includes a furin cleavage site mutation. In some aspects, the furin cleavage site eliminates biogenic processing of full-length Spike into S1 and S2 components. In further aspects, the furin cleavage site mutation includes 682RRAR685-to-682GSAG685. In some aspects, the modified SARS-CoV-2 Spike protein includes mutations present in a virulent strain of the SARS-CoV-2 virus (S^{delta}) a furin cleavage site mutation (CSM), diproline substitutions (2P), and a deleted COPI-binding CTT peptide replaced with a CTT peptide carrying a diacidic ER export signal (ΔC-ERES). In additional aspects, the modified SARS-CoV-2 spike protein is expressed on a cell surface about 500% more than an unmodified SARS-CoV-2 Spike protein.

Various embodiments of this disclosure are directed to an isolated polynucleotide including a sequence encoding a metabolic selectable marker (MSM), a linker peptide (LP), and an antibiotic resistance protein (AR). In some aspects, the MSM is a doxycycline-regulated Tet-on sequence rtTAv16 transcription factor or a glutamine synthetase. In further aspects, the MSM is glutamine synthetase. In additional aspects, the LP is a viral p2a peptide. In various aspects, the LP is a porcine teschovirus 2a peptide. Additionally, in some aspects, the AR is selected from the group consisting of BleoR, PuroR, PuroR2, BsdR, NeoR, and HygR. In further aspects, the AR is BleoR, PuroR, or PuroR2.

Various embodiments provided herein relate to an isolated polynucleotide sequence encoding a modified antigen including a non-Spike membrane-proximal external region (MPER), a transmembrane domain (TMD) and a carboxy-terminal tail (CTT). In some aspects, the MPER is a murine leukemia virus envelope glycoprotein (MLV MPER), a membrane-proximal external region of the human immunodeficiency virus type 1 (HIV MPER), or a vesicular stomatitis virus glycoprotein (VSVG). In further aspects, the MPER is MLV MPER. In additional aspects, the TMD is a type-1 exosomal membrane protein from an immunoglobulin superfamily. In some aspects, the TMD is IgSF2, IgSF3, or IgSF8. Further, in some aspects provided herein, the TMD is IgSF3 type-1 exosomal membrane protein (IgSF3 TMD). In various aspects, the CTT includes a diacidic ER export signal (ERES) and a Carajas virus G protein (CTT5). In additional aspects, the CTT includes an ERES and a fusion of CTT5 with a Golgi export signal of reovirus p14 (CTT6). In various aspects of the disclosure provided herein, the MPER is MLV, the TMD is IgSF3, and the CTT is CTT6. In certain aspects of the disclosure provided herein, the MPER is MLV, the TMD is IgFS8, and the CTT is CTT6. In some aspects the modified antigen is a SARS-CoV-2 Spike protein.

In further aspects of the disclosure provided herein, the SARS-CoV-2 Spike protein is a SARS-Cov2-Spike protein from a SARS-CoV-2 delta (Sdelta) virus. In additional aspects of the disclosure provided herein, the modified antigen is an extracellular domain of influenza hemagglutinin (HA). In further aspects, the modified antigen is an extracellular domain of vascular endothelial growth factor (VEGFR) and a constant region of the human immunoglobulin heavy chain (IgG Fc). In some aspects, the modified antigen is a modified alpha galactosidase A (GLA).

In some aspects, the disclosure provided herein relates to an exosome-based vaccine, wherein the vaccine includes an exosome including the modified antigen.

In some embodiments, the present disclosure relates to an expression vector wherein the coding region includes from a 5′ to a 3′ end: a) a first inverted tandem repeat (ITR-1) flanking b) a region including: a selectable marker system (SMS), a promoter, an exosome cargo protein (EC), a woodchuck hepatitis virus post-transcriptional regulatory element (WPRE) and a polyadenylation site (pAn); c) a second ITR (ITR-r) d) a Rous sarcoma virus long-terminal repeat (RSV); e) a Sleeping Beauty transposase SB100x; and f) a polyadenylation site (pAn). In some aspects, the SMS encodes a polypeptide including a glutamine synthase (GS) protein, a porcine teschovirus 2a peptide linker and an antibiotic resistance (AR) protein. In additional aspects, the SMS further includes a promoter. In further aspects, the EC is a modified SARS-CoV-2 Spike protein. In some aspects, the EC is a modified alpha galactosidase A (GLA). Also, in some aspects provided herein, the EC is an extracellular domain of vascular endothelial growth factor (VEGFR) and a constant region of a human immunoglobulin heavy chain (IgG Fc). According to various aspects in this disclosure, the EC is a heavy chain of trastuzumab and a light chain of trastuzumab.

In some embodiments, the methods described herein relate to a method of obtaining exosomes produced by a cell, including: a) introducing an isolated polynucleotide including TSPAN7/Y246A and a heterologous peptide into a cell in a first culture media; b) culturing the cells of a) in a second culture media; and c) harvesting an increased number of exosomes from the third culture media, wherein the number of exosomes is increased relative to a cell not including the polynucleotide sequence.

In some embodiments, the methods described herein relate to a method of delivering a heterologous peptide to an exosome, including introducing an isolated polynucleotide including TSPAN7/Y246A and a heterologous peptide into a cell and culturing the cell in a culture media, thus delivering the heterologous peptide to an exosome.

In some aspects, the methods described herein relate to a method, wherein the amount of exosomes produced by the cell is increased relative to a cell not having the polynucleotide.

In some embodiments, the methods described herein relate to a method for producing extracellular vesicles (“EVs”), including: (i) inserting the coding region for an exosome carrier protein (“ECP”) into an expression vector that is configured to drive the recombinant ECP expression; (ii) transfecting the expression vector into a cell line suitable for producing EVs; (iii) selecting and growing a transgenic cell line that expresses a high level of the recombinant ECP in culture media; and (iv) collecting EVs from the conditioned tissue culture media.

In some aspects of the methods described herein, the transgenic cell line that expresses a high level of the recombinant ECP in the step (iii) is a transgenic cell line that expresses the highest level of the recombinant ECP. In some aspects of the method described herein, the ECP is CD63/Y235A, CD9, or TSPAN7. In some aspects of the method described herein, the high-level expression of CD63/Y235A leads to approximately 5-fold increase in the EV production yield. In some aspects of the method described herein, the high-level expression of CD9 leads to approximately 10-fold increase in the EV production yield. In some aspects of the method described herein, the high-level expression of TSPAN7 leads to approximately 20-fold increase in the EV production yield. In some aspects of the method described herein, the EVs are exosomes or microvesicles. In some aspects of the method described herein, the cell line suitable for producing EVs is a 293F-derived cell.

In some embodiments, the present disclosure relates to an expression vector for producing EVs, including the coding region for an ECP. In some aspects, the present disclosure relates to an expression vector, wherein the ECP is CD63/Y235A, CD9, or TSPAN7. In some aspects, the present disclosure relates to an expression vector, wherein the EVs are exosomes or microvesicles.

In some embodiments, the present disclosure relates to a cell line for producing EVs, including the expression vector. In some aspects, the present disclosure relates to a cell line, wherein the EVs are exosomes or microvesicles.

### BRIEF DESCRIPTION OF THE DRAWINGS

FIG. 1 illustrates the antibiotic resistance (AR) gene test vectors. Fifteen distinct Sleeping Beauty transposon-containing vectors were created, each carrying a single transposon-carried gene in which the cytomegalovirus (CMV) enhancer/promoter was positioned to drive the expression of a bicistronic open reading frame (ORF) encoding (i) mCherry, (ii) a viral 2a peptide, and (iii) an AR protein. These AR proteins included untagged, ER50 degron-tagged, or ecDHFR degron-tagged forms of the five AR proteins BsdR, BleoR, PuroR, HygR, and NeoR. Following transfection of these vectors into 293F cells, expression of the Sleeping Beauty transposase SB100X is driven from the Rous sarcoma virus (RSV) long-terminal repeat, leading to mobilization of all sequences including and between the inverted tandem repeats (ITRs), from the vector and into one or more sites in the host cell genome, leading subsequently to expression of mCherry in a manner dependent upon integration site and integrant copy number, both of which can vary dramatically within the population of transgenic cells. WPRE is a woodchuck hepatitis virus post-transcriptional regulatory element that increases gene expression, and pAn is a polyadenylation site.

FIGS. 2A-2C illustrate that degron-tagging BsdR genes results in approximately fivefold higher expression of the linked recombinant protein mCherry. Flow cytometry measurements of mCherry expression levels (fluorescence brightness, arbitrary units) in polyclonal cell lines that were generated following transfection with transposons carrying the untagged BsdR ORF (FIG. 2A), ER50BsdR ORF (FIG. 2B), ecDHFRBsdR ORF (FIG. 2C) are also illustrated. Data are from three technical replicates of each cell line. Approximately 20,000 cells were assayed in each replicate; grey shows the background fluorescence of 293F cells, and the median, mean, and coefficient of variation (CV) from each replicate are shown to the right. Light purple shows mCherry expression in the BsdR cell line data, medium purple shows the ER50BsdR cell line data, and dark purple shows the ecDHFRBsdR cell line data. Data from 293F cells are shown in the box at the bottom right of the figure. ecDHFR is Escherichia coli dihydrofolate reductase; ER50 is estrogen receptor 50.

FIGS. 3A-3B are graphs illustrating the observation that ER50BleoR selects for twofold higher levels of linked mCherry expression. Flow cytometry histograms of mCherry expression levels (fluorescence brightness, arbitrary units) in the polyclonal cell lines selected using transposons carrying the untagged BleoR ORF (FIG. 3A), and the ER50BleoR ORF (FIG. 3B). Data are shown for three technical replicates of each cell line, involving 20,000 independent cell fluorescence measurements for each replicate, with gray showing the background fluorescence of 293F cells, and the median, mean, and CV from each replicate are shown to the right. Light chartreuse shows the BleoR cell line data, whereas dark chartreuse shows the ER50BleoR cell line data. Data from 293F cells are shown in the box at the bottom right of the figure. ER50 is estrogen receptor 50.

FIGS. 4A-4C illustrate how degron-tagging PuroR increases linked mCherry expression by 70%. Flow cytometry measurements of mCherry expression levels (fluorescence brightness, arbitrary units) in polyclonal cell lines selected via the untagged PuroR ORF (FIG. 4A), the ER50BleoR ORF (FIG. 4B), and the ecDHFRPuroR ORF (FIG. 4C). Data are shown for three technical replicates of each cell line, involving 20,000 independent cell fluorescence measurements for each replicate, with gray showing the background fluorescence of 293F cells, and the median, mean, and CV from each replicate are shown to the right. Light gray shows the PuroR-selected cell line data, medium gray shows the ER50PuroR-selected cell line data, and dark gray shows the ecDHFRPuroR-selected cell line data. Data from 293F cells are shown in the box at the bottom right of the figure. ecDHFR is Escherichia coli dihydrofolate reductase; ER50 is estrogen receptor 50.

FIGS. 5A-5C illustrate that degron tagging has only minimal effects on HygR-selected transgene expression. Flow cytometry measurements of mCherry expression levels (fluorescence brightness, arbitrary units) in the polyclonal cell lines generated following transfection with transposons carrying transgenes expressing the untagged HygR ORF (FIG. 5A), the ER50HygR ORF (FIG. 5B), and the ecDHFRHygR ORF (FIG. 5C). Data are shown for three technical replicates of each cell line, involving approximately 20,000 independent cell fluorescence measurements for each replicate, with grey showing the background fluorescence of 293F cells, and the median, mean, and CV from each replicate are shown to the right. Light cyan shows the HygR-selected cell line data, medium cyan shows the ER50HygR-selected cell line data, dark cyan shows the ecDHFRHygR-selected cell line data. Data from 293F cells are shown in the box at the bottom right of the figure. ecDHFR is Escherichia coli dihydrofolate reductase; ER50 is estrogen receptor 50.

FIGS. 6A-6C illustrate that degron tagging has only minimal effects on NeoR-selected transgene expression. Flow cytometry measurements of mCherry expression levels (fluorescence brightness, arbitrary units) in the polyclonal cell lines selected using transposons carrying the untagged NeoR ORF (FIG. 6A), the ER50NeoR ORF (FIG. 6B), and the ecDHFRNeoR ORF (FIG. 6C). Data are shown for three technical replicates of each cell line, involving 20,000 independent cell fluorescence measurements for each replicate, with gray showing the background fluorescence of 293F cells, and median, mean, and CV from each replicate are shown to the right. Light lavender shows the average data from the NeoR-selected cell line, medium lavender shows the average data from the ER50NeoR-selected cell line, and dark lavender shows the average data from the ecDHFRNeoR-selected cell line. Data from 293F cells are shown in the box at the bottom right of the figure. ecDHFR is Escherichia coli dihydrofolate reductase; ER50 is estrogen receptor 50.

FIG. 7A-7G illustrate experimental results showing that ER50BleoR selects for higher expression and improved exosome engineering. FIG. 7A illustrates line diagrams of Sleeping Beauty transposon vectors YA22 and YA24, which drive the expression of CD63/Y235A linked to the BleoR and ER50BleoR antibiotic resistance proteins, respectively. It should be noted that these bicistronic ORFs were expressed from the spleen focus-forming virus (SFFV) long terminal repeat (LTR), which appears to drive slightly higher transgene expression from integrated transgenes than the cytomegalovirus (CMV) enhancer/promoter elements. FIG. 7B-7D are images of immunofluorescence micrographs showing anti-CD63 fluorescent antibody staining of 293F cells (FIG. 7B), the zeocin-resistant 293F-derived cell line YA22 (FIG. 7C), and the zeocin-resistant 293F-derived cell line YA24 (FIG. 7D). Top panels are brightfield images, and bottom panels are anti-CD63 immunofluorescent images collected at the same exposure time for all three samples. The bar represents 150 μm. FIG. 7E illustrates immunoblots of cell lysates interrogated using (upper panel) a monoclonal antibody specific for CD63 and (lower panel) an antibody specific for HSP90. In an effort to accurately convey the difference in CD63 expression levels between 293F, F/YA22, and F/YA24 cells, an overexposed image of the immunoblot in this figure, though a nonsaturated exposure was used for subsequent quantification. FIG. 7F illustrates immunoblots of equal proportions of exosomes collected from the same triplicate cultures as in FIG. 7E, demonstrating that high-level expression of CD63/Y235A results in elevated levels of exosome-associated CD63 proteins. FIG. 7G is an image of a bar graph showing the amount of CD63 in cell and exosome lysates, with bar height denoting the average, error bars representing the standard error of the mean, asterisks denoting p value significance (**<0.005, ***<0.0005, ****<0.00005), and individual data points shown as points. Differences between the F/YA22 and F/YA24 samples were 2.1× for cell-associated CD63 and 3.5-fold for exosome-associated CD63. Numerical values were obtained by quantification of nonsaturated exposures of each immunoblot. ER50 is estrogen receptor 50; HSP90 is heat shock protein 90.

FIGS. 8A-8F illustrate binding of CP05-Cy5 to 293F, F/CD63^{-/-}, and F/YA24 cells. FIG. 8A is a diagram of the genomic DNA sequence in the vicinity of the Cas9/gRNA target site (SEQ ID NO: 1). Shaded sequence corresponds to the 3′ end of exon5, whereas the unshaded sequence corresponds to the 5′ end of intron 5. Underlined sequence denotes the guide RNA (gRNA) target site. FIG. 8B illustrates the DNA sequence of alleles 1 (SEQ ID NO: 2) and 2 (SEQ ID NO: 3) in the F/CD63^{-/-} cell line, resulting from Cas9/gRNA-mediated gene editing. FIG. 8C illustrates CD63 mRNA abundance in 293F and F/CD63^{-/-} cells, as determined by quantitative PCR (qRT-PCR). FIG. 8D illustrates flow cytometry histograms of (purple) F/CD63^{-/-} cells, (red) 293F cells, and (green) F/YA24 cells, each stained with the same FITC-labeled anti-CD63 monoclonal antibody. FIG. 8E and FIG. 8F illustrate flow cytometry measurements of CP05-Cy5 fluorescence staining of (purple) F/CD63^{-/-} cells, (red) 293F cells, and (green) F/YA24 cells stained with the CP05-Cy5 peptide at a concentration of 0.34 μM of CP05-Cy5 peptide (FIG. 8D) and at a concentration of 3.4 μM of CP05-Cy5 peptide (FIG. 8E).

FIG. 9 is an illustrative schematic representation of how choice of AR gene affects transgene expression. Gray bar represents the range of transgene expression within the entire population of transgenic cells in a transfected cell population, prior to addition of a selective antibiotic. Black, blue, and orange bars represent the range of transgene expression in polyclonal antibiotic-resistant cell lines selecting using AR proteins that have high, moderate, or low activity/stability, respectively. Black, blue, and orange arrows denote the threshold of transgene expression below which no cell can survive. Hatched bars represent the population of transgenic cells that will perish after the addition of selective antibiotic.

FIG. 10 is an illustrative schematic representation of the shared, stochastic model of exosome (exo) biogenesis. The secretion of exosome cargo proteins is a cargo-driven process that occurs (i) primarily at the plasma membrane and is (ii) inhibited by cargo protein endocytosis, driven by clathrin-mediated and actin-dependent endocytosis, such as the adapter protein complex (AP-2). However, (iii) inhibiting the endocytosis of a cargo protein will induce its exosomal secretion from the plasma membrane (PM), and (i′) syntenin induces CD63 budding by blocking its AP-2-mediated endocytosis. Once endocytosed, exosome cargoes will (iv) continue to drive extracellular vesicle (EV) budding, leading to the formation of intraluminal vesicles (ILVs) and their accumulation in the lumen of endolysosomal compartments. Although (v) most ILVs are delivered to lysosomes and degraded, it's clear that (vi) some ILVs are released as exosomes by endolysosomal exocytosis. Boxes with dashed outlines denote PM-localized cargoes that bud primarily from the PM yet (translucent dashed boxes) are nevertheless endocytosed at a low rate and loaded into ILVs. Non-dashed boxes denote endocytosed cargoes that carry an endocytosis signal (grey line), bud into ILVs, bud from the PM at a low rate when their endocytosis is efficient, yet bud primarily from the PM when their endocytosis is blocked.

FIG. 11 illustrates that Spike is not an exosomal protein, through anti-Spike and anti-CD9 immunoblots of cell and exosome (exo) lysates from 293F cells and 293F cells expressing wild type Spike (S^{W1}) or Spike with the D614G mutation (S^{D614G})

FIGS. 12A-12F illustrate that diproline (2P) and diacidic ER export signal (ERES) substitutions increase the cell surface expression of Spike. FIGS. 12A-12D illustrate anti-Spike flow cytometry scatter plots of surface-stained 293F cells (FIG. 12A) and 293F cells (FIGS. 12B-12C) transfected with plasmids designed to express Spike carrying the D614G mutation (S-D614G, FIG. 12B), Spike-D614G carrying the Spike D614G mutation and diproline substitutions (S-D614G-2P, FIG. 12C), and Spike carrying the D614G mutation, diproline substitutions (2P), and the ERES C-terminal tail substitutions (S-D614G-2P-ΔC-ERES, FIG. 12D). FIG. 12E is an illustration of a bar graph showing the integrated cell surface Spike expression of S-D614G, S-D614G-2P, and S-D614G-2P-ΔC-ERES. FIG. 12F is an illustration of an anti-Spike and anti-Hsp90 immunoblot of whole cell lysates of (left three lanes) control 293F cells, (center left three lanes) 293F cells expressing S-D614G, (center right three lines) 293F cells expressing S-D614G-2P, and (right three lanes)S-D614G-2P-ΔC-ERES. HSP90 is heat shock protein 90, used as a positive control,

FIGS. 13A-13G illustrate characterizations of S^{delta}-CSM-2P-ΔC-ERES display exosomes, where all of the selected Spike mutations present in the delta strain of the SARS-CoV-2 (T19R, G142D, D157-158, L452R, T478K, D614G, P681R, and D950N) are combined with a furin cleavage site mutation (CSM), a diproline (2P) substitution, and a C-terminal diacidic ER export signal (ΔC-ERES) substitution. FIG. 13A illustrates an immunoblot of S^{delta}-CSM-2P-ΔC-ERES display exosomes produced in three parallel trials, probed with antibodies to detect Spike (top panel) and CD9 proteins (bottom panel). FIGS. 13B-13G illustrate negative stain electron micrographs of 293F exosomes (FIGS. 13B and 13C) and Spike protein display exosomes (S^{delta}-DEX, FIGS. 13D-13G).

FIG. 14A-14D illustrate characterizations of Spike loading into exosomes by different type-1 anchor elements (T1A), comprising three different non-Spike membrane-proximal external regions (MPER), four different transmembrane domains (TMD), and six different carboxy-terminal tails (CTT). FIG. 14A illustrates a schematic of an exosome display protein (Spike) expressed N-terminal to a type-1 anchor comprised of a membrane proximal extracellular region (MPER), a transmembrane domain (TMD) and a C-terminal tail (CTT). FIG. 14B illustrates anti-Spike and anti-CD9 immunoblots of exosomes collected from cells expressing S^{delta}-2P-TMD3-ΔC-CTT5 proteins carrying three different MPER sequences: MPER2 (human immunodeficiency virus type 1 envelope (HIV ENV), Zwick et al., 2005), MPER3 (murine leukemia virus envelope (MLV ENV) Salamango et al., 2016), and MPER4 (vesicular stomatitis virus glycoprotein (VSVG), Rose et al., 1980). FIG. 14C illustrates anti-Spike and anti-CD9 immunoblots of exosomes collected from cells expressing S^{delta}-2P-ΔC-CTT5 proteins carrying four different TMD sequences: the transmembrane domain from unmodified Spike protein, and type-1 exosomal membrane proteins from the immunoglobulin superfamily IgSF2, IgSF3, and IgSF8. FIG. 14D Anti-Spike and anti-CD9 immunoblots of exosomes collected from cells expressing S^{delta}-2P-ΔC-ERES proteins carrying six different CTT sequences, including Carajas virus G protein (CTT5), and the fusion of CTT % with the Golgi export signal of reovirus p14 (CTT6).

FIG. 15A-15B illustrate a schematic diagram of a modified Tet-on expression plasmid and a modified Sleeping Beauty-based transgene delivery vector. FIG. 15A illustrates the single transgene of plasmid pJM1464, which drives the expression of a bicistronic ORF encoding the doxycycline-regulated rtTAv16 transcription factor linked to the viral p2a peptide and to the PuroR2 gene that encodes a novel puromycin acetyltransferase. The ORF is expressed from the spleen focus-forming virus (SFFV) long terminal repeat. The plasmid includes a woodchuck hepatitis virus post-transcriptional regulatory element (WPRE), and a polyadenylation site (pAn). FIG. 15B illustrates a three-gene, one plasmid Sleeping Beauty transposon vector modified from the pITRSB vector, in which the transposase SB100x is encoded by a gene flanking a transposon that carries a selectable marker gene, GS-2a-BleoR, driven by the elongation factor loa short (EFS) promoter, and a doxycycline-regulated gene in which the tetracycline responsive element 3G promoter (TRE3G) is driving expression of an ORF encoding the protein of interest. The rtAv16-p2a-PuroR transgene of plasmid pJM1464 (FIG. 15A) was replaced with a new marker gene encoding the selectable marker gene human glutamine synthetase (Glu Syn), the porcine teschovirus 2a peptide (p2a), and the antibiotic resistance gene BleoR. The vector includes inverted tandem repeats (ITR-I and ITR-r), a woodchuck hepatitis virus post-transcriptional regulatory element (WPRE), and polyadenylation sites (pAn).

FIGS. 16A-16B illustrate the observation that FtetP cells and the GS-2a-BleoR selectable marker gene increase the expression and exosomal secretion of Spike. FIG. 16A illustrates immunoblots of cell lysates interrogated using (upper panel) a monoclonal antibody specific for Spike and (lower panel) an antibody specific for HSP90. FIG. 16B illustrates immunoblots of exosome lysates interrogated using (upper panel) a monoclonal antibody specific for Spike and (lower panel) and antibody specific for CD9. FIGS. 16A and 16B illustrate immunoblots of FtetZ cells expressing Spike linked to the PuroR gene (FtetZ/PuroR, left three lanes, FIGS. 16A and 16B) and FtetP cells expressing Spike linked to the GS-2a-BleoR gene (FtetP/GS-2a-BleoR, right three lanes, FIGS. 16A and 16B). n=3.

FIGS. 17A-17B illustrate superior loading of Spike into the exosome membrane using T1EMAv1 and T1EMAv2 exosome membrane anchors. T1EMAv1 combines the murine leukemia virus envelope glycoprotein (MLV MPER), the IgSF3 type-1 exosomal membrane protein (IgSF3 TMD), and the Golgi export signal of reovirus p14 (CTT6), while T1EMAv2 combines the MLV MPER, IgFS8 TMD, and CTT6. Anti-Spike, anti-Hsp90, and anti-CD9 immunoblots of cell lysates (FIG. 17A) and exosome lysates (FIG. 17B) collected from FtetP cells expressing S^{delta}-CSM-2P-ΔC-ERES (-ΔC-ERES, left three lines), S^{delta}-CSM-2P-T1EMAv1 (T1Av1, middle three cell lines), and S^{delta}-CSM-2P-T1EMAv2 (T1Av2, right three cell lines).

FIGS. 18A-18B illustrate that the T1EMAv2 anchor peptide directs more hemagglutinin (HA) into the exosome membrane than the PTGFRN anchor peptide. FIG. 18A and FIG. 18B illustrate the results of immunoblots of cell lysates (FIG. 18A) and exosome lysates (FIG. 18B) generated by FtetP cells expressing HA-PTGFRN, HA-T1EMAv1, and HA-T1EMAv2.

FIGS. 19A-19C illustrate experimental results showing that the T1EMAv2 anchor peptide loads the extracellular domain of vascular endothelial growth factor receptor fused to the constant region of the human immunoglobulin heavy chain (Fc domain of human IgG) (VEGFR-Fc), the lysosomal enzyme alpha galactosidase A (GLA), and the heavy chain of the HER2-inhibiting monoclonal antibody Trastuzumab (Tz-mAb) into the exosome membrane. FIGS. 19A-19C illustrate several immunoblots of exosome and cell lysates generated by FtetP cells expressing VEGFR-Fc-T1EMAv2 (FIG. 19A), GLA-T1EMAv2 (GLA, FIG. 19B), and Trastuzumab-HC-T1EMAv2 co-expressed with a third gene encoding an unaltered form of the trastuzumab light chain (TZ-mAB, FIG. 19C), probed with antibodies specific for human IgG (FIGS. 19A and 19C), and GLA (FIG. 19B).

FIGS. 20A-20H depict elevated exosome production by genetically modified 293F cells. (FIG. 20A) Bar graph showing average exosome yield by 239F (1×), F/YA24 (4.6×), F/YA73 (9×), and F/YA71 (20×) cell lines (n=3), as determined by nanoparticle tracking analysis (NTA). (FIGS. 20B-20D) Transmission electron micrographs of exosomes purified from 293F, F/YA73, and F/YA71 cells. (FIGS. 20E-20H) NTA histograms of exosomes purified from the cell lines 239F (135 nm average diameter), F/YA24 (105 nm average diameter), F/YA73 (120 nm average diameter), and F/YA71 (102 nm average diameter).

FIG. 21 depicts the structure of a modified type-1 exosome membrane protein. The protein includes a signal sequence, a region of interest that encodes a peptide, protein, or antigen, and a trans-membrane protein T1EMA. T1EMAv1, T1EMAv2, and T1EMAv3 have distinct DNA sequences, with T1EMAv3 including an optimized MPER, an optimized TMD, and an optimized CTT.

FIG. 22A illustrates exosome yield due to expression of various protein constructs. Expression of CD63-Y235A leads to high yield of exosomes, expression of CD9 leads to an even higher yield, and high level expression of TSPAN7 leads to about a 20-fold increase in the numbers of exosomes produced per cell, or per mL of cell culture, above the numbers of exosomes produced per cell when TSPAN7 is not expressed (293F). Expression of TSPAN7/Y246A leads to an even higher increase in the numbers of exosomes produced per cell, to about 30-fold above the numbers of exosomes produced per cell when TSPAN7/Y246A is not expressed.

FIG. 22B depicts the topology of the transmembrane protein TSPAN7, with its N-terminus (N) and C-terminus (C) in the inside of a membrane; for example, on the cytosolic side of a membrane, or in an exosome lumen. The first (EC1) and second (EC2) extracellular loops are on the outside of a membrane; for example, on the extracellular side of a membrane, or in the extracellular space of an exosome.

FIGS. 22C and 22D depict a TSPAN7/Y236A platform for loading peptides, proteins, and antigens into the lumen of exosomes. In FIG. 22C, the TSPA7Y246A construct is located adjacent to a desired peptide, protein, or antigen. The desired peptide, protein, or antigen is located at the N-terminal or at the C-terminal of the transmembrane protein TSPAN7/Y246A, resulting in the intracellular location of the desired peptide. In FIG. 22D, the desired peptide, protein, or antigen is inserted into the first (FIG. 22B, EC1) or second (FIG. 22B, EC2) extracellular loop of TSPAN7/Y246A, resulting in the extracellular display of the desired peptide.

### DETAILED DESCRIPTION OF THE INVENTION

The production of engineered exosomes of defined content function and yield requires tools and technologies that allow for the selective loading of proteins on the exosome surface or in the exosome membrane. The present disclosure describes polypeptide sequences capable of accomplishing both at a high level of efficiency. Specifically, type-1 exosome membrane anchors (T1EMAv1, T1EMAv2) that load heterologous proteins into the exosome membrane so that the heterologous protein located on the outer surface of the exosomes, and a polytopic membrane protein anchor (TSPAN7/Y246A) that can loads proteins into the loumen of exosomes are described herein.

The invention is based on the finding that tagging genes with a destabilization domain can select for higher transgene expression in eukaryotic cell lines. This enables higher selectivity of antibiotic resistance genes following transfection of vectors into cells. It also allows higher expression of exosome-related proteins, which has applications in cell engineering. Additionally, the present disclosure provides that high-level expression of recombinantly-expressed, high-efficiency exosome carrier proteins (“ECPs”) leads to significant increases in exosome production yield (number of exosomes/mL of conditioned media collected from exosome-producing cell line).

Exosomes are the only bionormal nanovesicle and therefore hold high potential as safe and non-toxic vesicles for delivery of vaccines and therapeutics. To help realize this potential, the present disclosure includes a roadmap for exosome engineering based on observations that exosome biogenesis is a cargo-driven process that occurs primarily at the plasma membrane. This disclosure validates a method for the production of SARS-CoV-2 Spike display exosomes. Specifically, the present disclosure shows that the loading of type-1 membrane proteins into the exosome membrane is facilitated by a multi-step process that involves (i) minimizing its trafficking to intracellular compartments, (ii) fusion to synthetic type-1 anchor peptides that optimize protein stability and transport them to the plasma membrane, and (iii) maximizing its expression by use of an optimized cell transgenesis and recombinant protein expression technology. Taken together, these results demonstrate that mechanism-based exosome engineering allows for improved production of exosome display vesicles of high clinical potential, a conclusion further supported by the production of engineered exosomes decorated with influenza hemagglutinin (HA), alpha galactosidase A, and the receptor for vascular endothelial growth factor.

Intracellular membrane trafficking directs the movement of cellular cargo, including proteins, using membrane-bound transport vesicles. Protein transport is mediated in part by signal peptides that direct proteins to and from the Golgi complex, the endoplasmic reticulum (ER), the plasma membrane (PM), lysosomes, endosomes, secretory granules, and other vesicles and membranes. Signal secretion peptides can cause a protein to be secreted by the cell, displayed on the plasma membrane, or displayed on extracellular vehicles such as exosomes. Examples of signal peptides include diacidic ER export signal peptides (ERES), also found in vesicular stomatitis virus glycoprotein (VSVG). Peptides in other viral proteins also act as signal peptides, such as the membrane-proximal external region of the human immunodeficiency virus type 1 (HIV ENV), and the murine leukemia virus envelope glycoprotein (MLV ENV).

Protein engineering designed to direct proteins from the ER to the PM can be optimized if competing localization signals are disrupted. For example, type-1 membrane proteins are proteins that have a single membrane-spanning region, a C-terminal tail (CTT) in the cytosolic cellular region, and an N-terminus in the extracellular or luminal region. Type-1 membrane proteins are often retained in the ER and potentially degraded by the ER-associated protein degradation pathway due to oligomerization of monomers. Disruption of this process through protein engineering, such as with the methods presented herein, can optimize transport of modified proteins to the PM and to exosomes or other extracellular vesicles.

To further direct membrane traffic, the proteins COPI and COPII coat intracellular vesicles and help direct vesicles from the Golgi to the ER (COPI), or from the ER to the Golgi (COPII). COPI-binding CTT regions recruit COPI proteins to the vesicles carrying those proteins, and direct recycling of such proteins from the Golgi to the ER. On the other hand, COPII-binding CTT regions recruit COPII proteins to vesicles and direct export from the ER to the Golgi. Syntenin clathrin, and actin are also involved in protein trafficking, especially endocytosis and the production of exosomes.

As used herein, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, “and/or” refers to and encompasses any and all possible combinations of one or more of the associated listed items, as well as the lack of combinations when interpreted in the alternative (or).

The terms “about” and “approximate”, as used herein when referring to a measurable value such as an amount, dose, time, temperature, activity, level, number, frequency, percentage, dimension, size, amount, weight, position, length and the like, is meant to encompass variations of ±15%, ±10%, ±5%, ±1%, ±0.5%, or even ±0.1% of the specified amount, dose, time, temperature, activity, level, number, frequency, percentage, dimension, size, amount, weight, position, length and the like. In instances in which the terms “about” and “approximate” are used in connection with the location or position of regions within a reference polypeptide, these terms encompass variations of ± up to 20 amino acid residues, up to 15 amino acid residues, ± up to 10 amino acid residues, ± up to 5 amino acid residues, up to 4 amino acid residues, ± up to 3 amino acid residues, ± up to 2 amino acid residues, or even ±1 amino acid residue.

The term “clone” refers to a group of identical cells that share a common ancestry, e.g., they are derived from the same cell.

The term “derived from” as in “A is derived from B” means that A is obtained from B in such a manner that A is not identical to B. For example, if a destabilization domain (DD) is “derived from” the Escherichia coli dihydrofolate reductase (ecDHFR), that means the degradation domain of the ecDHFR is obtained from ecDHFR, thereby providing ecDHFR(DD). Accordingly, the term “ecDHFRBsdR” refers to BsdR which has been derivatized by appending the degradation domain of the ecDHFR to the N-terminus of BsdR. The term “polycistronic” (e.g., “bicistronic”) refers to a nucleic acid molecule, e.g., mRNA, which, upon translation, produces a plurality of polypeptides. A plurality of polypeptides can be produced by, for example, inclusion of a plurality of open reading frames, or a single reading frame comprising a self-cleaving peptide, such as viral 2A peptide.

The term “destabilization domain” (DD) refers to a protein, polypeptide or amino acid sequence that modulates the stability of a protein when operably connected to, linked to, or fused to (e.g., as a fusion component of), the protein. For example, the term “destabilization domain” (DD), refers to a protein domain that is unstable and degraded in the absence of ligand, but whose stability is rescued by binding to a high affinity cell-permeable ligand. Destabilization domains (DDs) can be fused or linked to a target protein and can convey its destabilizing property to the protein of interest, causing protein degradation. DDs render the attached protein of interest unstable in the absence of a DD-binding ligand such that the protein is rapidly degraded by the ubiquitin-proteasome system of the cell. However, when a specific small molecule ligand binds its intended DD as a ligand binding partner, the instability is reversed and some level of protein function is restored. The conditional nature of DD stability allows a rapid and non-perturbing switch from stable protein to unstable substrate for degradation. Such a destabilization domain may or may not require the interaction of another protein for modulating stability of the protein. Non-limiting examples of DDs include structurally unstable protein domains derived from Escherichia coli dihydrofolate reductase (DHFR), as described in Iwamoto M, Björklund T, Lundberg C, Kirik D, Wandless T J. Chem Biol. 2010; 17:981-988, and the human estrogen receptor (ER50), as described in Miyazaki Y, Imoto H, Chen L C, Wandless T J. J Am Chem Soc. 2012; 134:3942-3945, as well as Maji B, Moore C L, Zetsche B, Volz S E, Zhang F, Shoulders M D, Choudhary A. Multidimensional chemical control of CRISPR-Cas9. Nat Chem Biol. 2017 Jan; 13(1):9-11, the contents of which are each incorporated herein by reference in their entirety.

A destabilization domain can include KEN, Cyclin A, UFD domain/substrate, ubiquitin, PEST sequences, destruction boxes and hydrophobic stretches of amino acids. Exemplary destabilization domains include ubiquitin and homologs thereof, particularly those comprising mutations that prevent, or significantly reduce, the cleavage of ubiquitin multimers by a-NH-ubiquitin protein endoproteases.

As used herein, the term “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

The term “expression control sequence” refers to a nucleotide sequence that regulates transcription and/or translation of a nucleotide sequence operatively linked thereto. Expression control sequences include, but are not limited to, promoters, enhancers, repressors (transcription regulatory sequences) and ribosome binding sites (translation regulatory sequences).

As used herein the terms “antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. “Antibody,” as used herein, encompasses any polypeptide comprising an antigen-binding site regardless of the source, species of origin, method of production, and characteristics. Antibodies include natural or artificial, mono- or polyvalent antibodies including, but not limited to, polyclonal, monoclonal, multispecific, human, humanized or chimeric antibodies, single chain antibodies, and antibody fragments. “Antibody fragments” include a portion of an intact antibody, preferably the antigen binding or variable region of the intact antibody. Examples of antibody fragments include Fab, Fab′ and F(ab′)2, Fc fragments or Fc-fusion products, single-chain Fvs (scFv), disulfide-linked Fvs (sdfv) and fragments including either a VL or VH domain; diabodies, tribodies and the like (Zapata et al. Protein Eng. 8(10):1057-1062 [1995]). The term “antibody,” as used herein, refers to immunoglobulin molecules and immunologically active portions of immunoglobulin molecules, i.e., molecules that contain an antigen binding site that immunospecifically binds an antigen.

Experimentally, antibodies can be cleaved with the proteolytic enzyme papain, which causes each of the heavy chains to break, producing three separate antibody fragments. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding but is important in later processes involved in ridding the body of the antigen.

Antigen: An “antigen” is a molecule or entity to which an antibody binds. In some embodiments, an antigen is or comprises a polypeptide or portion thereof. In some embodiments, an antigen is a portion of an infectious agent that is recognized by antibodies. In some embodiments, an antigen is an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In general, an antigen may be or include any chemical entity such as, for example, a small molecule, a nucleic acid, a polypeptide, a carbohydrate, a lipid, a polymer other than a biologic polymer (e.g., other than a nucleic acid or amino acid polymer) etc. In some embodiments, an antigen is or comprises a polypeptide. In some embodiments, an antigen is or comprises a glycan.

As used herein, the term “nucleic acid” or“oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid is isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be employed for introduction into, i.e., transfection of, cells, in particular, in the form of RNA which is prepared by in vitro transcription from a DNA template. The RNA can moreover be modified before application by stabilizing sequences, capping, and polyadenylation.

Generally, nucleic acid is extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; U.S. Pub. 2010/0285578; and U.S. Pub. 2002/0190663.

Isolated nucleic acid sequences, or alternatively, “codon-optimized” sequences of nucleic acid sequences of interest, modified to provide the sequences with preferred optimized characteristics, are provided. Such characteristics may include transcription, translation, post-translational modification, stability of the encoded protein, etc. When two or more nucleic acid sequences are included in a single vector or construct, they are in operable linkage, such that the sequences are properly encoded and expressed. In some embodiments, an expression vector can include nucleic acid, a polynucleotide sequence of which can encode a selectable marker (SM) protein that is operably linked to a protein of interest (POI) (e.g., an antibiotic resistance gene). In some embodiments, a nucleic acid is operably linked to an expression control sequence (e.g., a promoter). As used herein, a nucleotide sequence is “operably linked” with an expression control sequence when the expression control sequence functions in a cell to regulate transcription of the nucleotide sequence. This includes promoting transcription of the nucleotide sequence through an interaction between a polymerase and a promoter.

The term “open reading frame” (ORF) refers to a nucleotide sequence, typically positioned between a start codon and a stop codon, that has the ability to be translated into a polypeptide.

As used herein, a nucleotide sequence is “operably linked” with an expression control sequence when the expression control sequence functions in a cell to regulate transcription of the nucleotide sequence. This includes promoting transcription of the nucleotide sequence through an interaction between a polymerase and a promoter.

The terms “peptide”, “polypeptide” and “protein” are used interchangeably herein, and refer to any chain of at least two amino acids linked by a covalent chemical bound. As used herein, a peptide can refer to the complete amino acid sequence coding for an entire protein or to a portion thereof. A “protein coding sequence,” or a sequence that “encodes” a particular polypeptide or peptide, is a nucleic acid sequence that is transcribed, in the case of DNA, and is translated, in the case of mRNA, into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

The term “extracellular vesicle” (EV) refers to lipid bilayer-delimited particles that are naturally released from cells. EVs range in diameter from around 20-30 nanometers to about 10 microns or more. EVs can include proteins, nucleic acids, lipids and metabolites from the cells that produced them. EVs include primarily exosomes, which are about 30 to about 150 nm, have the same topology and are enriched in exosome marker proteins, and also larger EVs such as microvesicles that are greater than 200 nm, but usually less than 500 nm. Preparations of small EVs and exosomes and large EVs and microvesicles are often contaminated by extracellular particles (EPs). EPs lack a membrane bilayer but can be of similar size to EVs. Common EPs include lipoprotein particles, exomeres, non-enveloped virus particles, and aggregated protein particles released by necrotic cells.

The contents of exosomes depend, in part, on the character of the cells that produce them. Cells can be genetically modified to configure exosomes produced by them. Fang et al., (PLOS, June 2007 vol. 5:1267-1283) describe methods of engineering proteins to preferentially target them toward exosomes. It was observed that (1) addition of both monoclonal mouse IgG to CD43 and polyclonal anti-mouse IgG antibodies were sufficient to induce the sorting of CD43 to exosomes, (2) addition of a plasma membrane anchor was sufficient to target a protein to exosomes, (3) a synthetic cargo comprised of a plasma membrane anchor and two heterologous oligomerization domains (Acyl-LZ-DsRED) was sorted to exosomes, (4) highly oligomeric, plasma membrane-associated retroviral Gag proteins (from EIAV, HTLV-1, RSV, MLV, MPMV, and HERV-K) were all sorted to ELDs and exosomes, and (5) a pair of heterologous oligomerization domains was necessary and sufficient to target HIV Gag to ELDs and exosomes. Elements, such as these, that traffic proteins to EVs, are referred to as “EV-trafficking elements.” Accordingly, any protein of interest can be modified in this way to traffic the protein towards exosomes.

Extracellular vesicles can be referred to herein as “delivery vehicles.” An extracellular vesicle can carry a cargo, which can be a protein of interest (POI) or a nucleic acid of interest (NAOI). The cargo molecule can be present within the lumen of the delivery vehicle or on its surface. The protein of interest can be a protein that is naturally produced by a cell that generates a delivery vehicle, or it can be a recombinant protein, including a non-naturally occurring protein, such as a fusion protein. The POI can be a viral protein, e.g., capable of eliciting an immune response. Nucleic acids include, without limitation, DNA and RNA. RNA can be mRNA. When delivered to a target cell, mRNA may be expressed as protein and presented on the cell surface to elicit an immune response. Nucleic acids are typically incorporated into EVs by contacting the EVs and the nucleic acid in the presence of a chemical lipofection reagent. The chemical lipofection reagent can be a polycationic lipid. In some embodiments, the chemical lipofection reagent is an mRNA lipofection reagent, or an mRNA transfection reagent, e.g., Lipofectamine® MessengerMAX™, Lipofectamine® 2000, Lipofectamine® 3000.

Exosomes are defined herein as all small, secreted vesicles of approximately 20-150 nm that are released by mammalian cells and made either by budding into endosomes or by budding from the plasma membrane of a cell. Exosomes can range in size from approximately 20-150 nm in diameter. In some cases, they have a characteristic buoyant density of approximately 1.1-1.2 g/mL, and a characteristic lipid composition. Their lipid membrane is typically rich in cholesterol and contains sphingomyelin, ceramide, lipid rafts and exposed phosphatidylserine. Exosomes express certain marker proteins, such as integrins and cell adhesion molecules, but generally lack markers of lysosomes, mitochondria, or caveolae. In some embodiments, the exosomes contain cell-derived components, such as but not limited to, proteins, DNA, and RNA (e.g., microRNA and noncoding RNA). In some embodiments, exosomes are obtained from cells obtained from a source that is allogeneic, autologous, xenogeneic, or syngeneic with respect to the recipient of the exosomes.

Exosomes are collected, concentrated and/or purified using methods known in the art. For example, differential centrifugation has become a leading technique wherein secreted exosomes are isolated from the supernatants of cultured cells. This approach allows for separation of exosomes from larger extracellular vesicles and from most non-particulate contaminants by exploiting their size. Exosomes are prepared as described in a wide array of papers, including but not limited to, Fordjour et al., “A shared pathway of exosome biogenesis operates at plasma and endosome membranes”, bioRxiv, preprint posted Feb. 11, 2019, at https://www.biorxiv.org/content/10.1101/545228v1; Booth et al., “Exosomes and HIV Gag bud from endosome-like domains of the T cell plasma membrane”, J Cell Biol., 172:923-935 (2006); and, Fang et al., “Higher-order oligomerization targets plasma membrane proteins and HIV gag to exosomes”, PLoS Biol., 5:e158 (2007). Exosomes using a commercial kit such as, but not limited to the ExoSpin™ Exosome Purification Kit, Invitrogen® Total Exosome Purification Kit, PureExo® Exosome Isolation Kit, and ExoCap™ Exosome Isolation kit. Methods for isolating exosome from stem cells are found in, e.g., Tan et al., Journal of Extracellular Vesicles, 2:22614 (2013); Ono et al., Sci Signal, 7(332):ra63 (2014) and U.S. Application Publication Nos. 2012/0093885 and 2014/0004601. Methods for isolating exosome from cardiosphere-derived cells are found in, e.g., Ibrahim et al., Exosomes as critical agents of cardiac regeneration triggered by cell therapy, Stem Cell Reports, 2014. Specific methodologies include ultracentrifugation, density gradient, HPLC, adherence to substrate based on affinity, or filtration based on size exclusion.

Size exclusion allows for their separation from biochemically similar but biophysically different microvesicles, which possess larger diameters of up to 1,000 nm. Differences in flotation velocity further allows for separation of differentially sized exosomes. In general, exosome sizes will possess a diameter ranging from 30-200 nm, including sizes of 40-100 nm. Further purification may rely on specific properties of the particular exosomes of interest. This includes, e.g., use of immunoadsorption with a protein of interest to select specific vesicles with exoplasmic or outward orientations.

Among current methods, e.g., differential centrifugation, discontinuous density gradients, immunoaffinity, ultrafiltration and high-performance liquid chromatography (HPLC), differential ultracentrifugation is the most commonly used for exosome isolation. This technique utilizes increasing centrifugal force from 2000×g to 10,000×g to separate the medium- and larger-sized particles and cell debris from the exosome pellet at 100,000×g. Centrifugation alone allows for significant separation/collection of exosomes from a conditioned medium, although it is insufficient to remove various protein aggregates, genetic materials, particulates from media and cell debris that are common contaminants. Enhanced specificity of exosome purification may deploy sequential centrifugation in combination with ultrafiltration, or equilibrium density gradient centrifugation in a sucrose density gradient, to provide for the greater purity of the exosome preparation (flotation density 1.1-1.2 g/mL) or application of a discrete sugar cushion in preparation.

Importantly, ultrafiltration is used to purify exosomes without compromising their biological activity. Membranes with different pore sizes—such as 100 kDa molecular weight cutoff (MWCO) and gel filtration to eliminate smaller particles—have been used to avoid the use of a nonneutral pH or non-physiological salt concentration. Currently available tangential flow filtration (TFF) systems are scalable (to >10,000 L), allowing one to not only purify, but to concentrate the exosome fractions, and such approaches are less time consuming than differential centrifugation. HPLC can also be used to purify exosomes to more uniformly sized particle preparations and to preserve their biological activity as the preparation is maintained at a physiological pH and salt concentration. Other chemical methods exploit differential solubility of exosomes for precipitation techniques, such as addition of volume-excluding polymers (e.g., polyethylene glycols (PEGs)), possibly combined with additional rounds of centrifugation or filtration. For example, a precipitation reagent, ExoQuick®, is added to conditioned cell media to quickly and rapidly precipitate a population of exosomes, although re-suspension of pellets prepared via this technique may be difficult. Flow field-flow fractionation (F1FFF) is an elution-based technique that is used to separate and characterize macromolecules (e.g., proteins) and nano- to micro-sized particles (e.g., organelles and cells), which has been successfully applied to fractionate exosomes from culture media.

Beyond these techniques relying on general biochemical and biophysical features, focused techniques may be applied to isolate specific exosomes of interest. These include relying on antibody immunoaffinity to certain exosome-associated antigens. As described, exosomes further express the extracellular domain of membrane-bound receptors at the surface of the membrane. This presents a ripe opportunity for isolating and segregating exosomes in connections with their parental cellular origin, based on a shared antigenic profile. Conjugation to magnetic beads, chromatography matrices, plates or microfluidic devices allows isolation of specific exosome populations of interest, which may be related to their production from a parent cell of interest, or to their associated cellular regulatory state. Other affinity-capture methods use lectins, which bind to specific saccharide residues on the exosome surface.

The term “selectable marker” (SM) refers to a gene introduced into a cell that confers a trait suitable for artificial selection. Non-limiting examples of selectable markers include antibiotic resistance markers such as BleoR (zeocin resistance), PuroR (puromycin resistance), PuroR2 (puromycin resistance), HygR (hygromycin resistance), NeoR (G418 resistance), and BsdR (blasticidin resistance). Additional selectable markers can include a protein sharing 50%, 55%, 60%, 65% or more amino acid sequence identity to a selectable marker protein. Such markers are useful for selecting stable transformants in culture.

SARS-CoV-2 is a member of a large family of viruses called coronaviruses. These viruses can infect people and some animals. SARS-CoV-2 was first known to infect people in 2019. The virus spreads from person to person through droplets released when an infected person coughs, sneezes, or talks. It may also be spread by touching a surface with the virus on it and then touching one's mouth, nose, or eyes, but this is less common. Research is being done to treat COVID-19 and to prevent infection with SARS-CoV-2. Also called severe acute respiratory syndrome coronavirus 2. Each SARS-CoV-2 virion has four structural proteins, known as the S (spike), E (envelope), M (membrane), and N (nucleocapsid) proteins; the N protein holds the RNA genome, and the S, E, and M proteins together create the viral envelope. Coronavirus S proteins are glycoproteins and also type I membrane proteins (membranes containing a single transmembrane domain oriented on the extracellular side). They are divided into two functional parts (S1 and S2). In SARS-CoV-2, the spike protein is the protein responsible for allowing the virus to attach to and fuse with the membrane of a host cell; specifically, its S1 subunit catalyzes attachment, the S2 subunit fusion. SARS-CoV-2 has sufficient affinity to the receptor angiotensin converting enzyme 2 (ACE2) on human cells to use them as a mechanism of cell entry. ACE2 acts as the receptor for SARS-CoV-2, with SARS-CoV-2 having a higher affinity to human ACE2 than the original SARS virus.

The term “coronavirus” or “CoV” refers to any virus of the coronavirus family, including but not limited to SARS-CoV-2, MERS-CoV, and SARS-CoV. SARS-CoV-2 refers to the newly-emerged coronavirus which was identified as the cause of a serious outbreak starting in Wuhan, China, and which is rapidly spreading to other areas of the globe. SARS-CoV-2 has also been known as 2019-nCoV and Wuhan coronavirus. It binds via the viral spike protein to human host cell receptor angiotensin-converting enzyme 2 (ACE2). The spike protein also binds to and is cleaved by TMPRSS2, which activates the spike protein for membrane fusion of the virus.

The term “CoV-S”, also called “S” or “S protein” refers to the spike protein of a coronavirus, and can refer to specific S proteins such as SARS-CoV-2-S, MERS-CoV S, and SARS-CoV S. The SARS-CoV-2-Spike protein is a 1273 amino acid type I membrane glycoprotein which assembles into trimers that constitute the spikes or peplomers on the surface of the enveloped coronavirus particle. The protein has two essential functions, host receptor binding and membrane fusion, which are attributed to the N-terminal (S1) and C-terminal (S2) halves of the S protein. CoV-S binds to its cognate receptor via a receptor binding domain (RBD) present in the S1 subunit. The term “CoV-S” includes protein variants of CoV spike protein isolated from different CoV isolates as well as recombinant CoV spike protein or a fragment thereof. The term also encompasses CoV spike protein or a fragment thereof coupled to, for example, a histidine tag, mouse or human Fc, or a signal sequence such as ROR1.

The term “coronavirus infection” or “CoV infection,” as used herein, refers to infection with a coronavirus such as SARS-CoV-2, MERS-CoV, or SARS-CoV. The term includes coronavirus respiratory tract infections, often in the lower respiratory tract. Symptoms can include high fever, dry cough, shortness of breath, pneumonia, gastro-intestinal symptoms such as diarrhea, organ failure (kidney failure and renal dysfunction), septic shock, and death in severe cases.

As used herein, the term “receptor-binding domain” is a short immunogenic fragment from a virus that binds to a specific endogenous receptor sequence to gain entry into host cells. Specifically, these refer to a part of the ‘spike’ glycoprotein (S-domain) which is needed to interact with endogenous receptors to facilitate membrane fusion and delivery to the cytoplasm. Typically, the S-domain is also the site of neutralizing antibodies.

The term “transgene” refers to a gene in a cell or organism that is not native to the at cell or organism, typically incorporated naturally or by any of a number of genetic engineering techniques.

The term “transposable element” or “transposon”, as used herein, refers to DNA sequences that are excised from one location in a genome and inserted into another location of the same or a different genome. Through genetic engineering, transposable elements enable insertion of novel DNA sequences into bacteria, viruses, plasmids, plants, animals, etc. Some transposable elements require reverse transcription for insertion (retrotransposons), and some do not (DNA transposons such as those used in the “Sleeping Beauty” vector system).

The term “vector”, “expression vector”, or “plasmid DNA” is used herein to refer to a recombinant nucleic acid construct that is manipulated by human intervention. A recombinant nucleic acid construct can contain two or more nucleotide sequences that are linked in a manner such that the product is not found in a cell in nature. In particular, the two or more nucleotide sequences are operatively linked, such as one or more genes encoding one or more proteins of interest, one or more protein tags, functional domains and the like. The expression vector of the invention can include regulatory elements controlling transcription generally derived from mammalian, microbial, viral or insect genes, such as an origin of replication, to confer to the vector the ability to replicate in a host, and a selection gene may additionally be incorporated, encoding, for example, a selectable marker (SM) protein to facilitate recognition of transformants. Those of skill in the art can select a suitable regulatory region to be included in such a vector, depending on the host cell used to express the gene(s). For example, the expression vector can include one or more promoters, operably linked to the nucleic acid of interest, or a gene of interest (GOI) capable of facilitating transcription of genes in operable linkage with the promoter. Several types of promoters are well known in the art and suitable for use with the present invention. The promoter can be constitutive or inducible.

Additional regulatory elements that may be useful in vectors include, but are not limited to, polyadenylation sequences, translation control sequences (e.g., an internal ribosome entry segment, IRES), enhancers, introns, and the like. Such elements may not be necessary, although they may increase expression by affecting transcription, stability of the mRNA, translational efficiency, and the like. Such elements can be included in a nucleic acid construct as desired to obtain optimal expression of the nucleic acids in the cell(s). Sufficient expression, however, may sometimes be obtained without such additional elements. Vectors also can include other elements. For example, a vector can include a nucleic acid that encodes a signal peptide such that the encoded polypeptide is directed to a particular cellular location (e.g., a signal secretion sequence to cause the protein to be secreted by the cell), or it can include a nucleic acid that encodes a selectable marker. Non-limiting examples of selectable markers include doxycycline, puromycin, adenosine deaminase (ADA), aminoglycoside phosphotransferase (neo, G418, APH), dihydrofolate reductase (DHFR), hygromycin-B-phosphotransferase, thymidine kinase (TK), and xanthin-guanine phosphoribosyltransferase (XGPRT). Such markers are useful for selecting stable transformants in culture. Non-limiting examples of vectors suitable for use for the expression of high levels of recombinant proteins of interest include those selected from baculovirus, phage, plasmid, phagemid, cosmid, fosmid, bacterial artificial chromosome, viral DNA, Pl-based artificial chromosome, yeast plasmid, transposon, and yeast artificial chromosome. For example, the viral DNA vector can be selected from vaccinia, adenovirus, foul pox virus, pseudorabies and a derivative of SV40. Non-limiting examples of suitable bacterial vectors include pQE70TM, pQE60TM, pQE-9TM, pBLUESCRIPTTM SK, pBLUESCRIPTTM KS, pTRC99aTM, pKK223-3TM, pDR540TM, PACTM and pRIT2TTM. Non-limiting examples of suitable eukaryotic vectors include pWLNEOTM, pXTITM, pSG5TM, pSVK3TM, pBPVTM, pMSGTM, and pSVLSV40TM. Non-limiting examples of suitable eukaryotic vectors include pWLNEOTM, pXTITM, pSG5TM, pSVK3TM, pBPVTM, pMSGTM, and pSVLSV40TM. One type of vector is a genomic integrated vector which can become integrated into the chromosomal DNA of the host cell. Another type of vector is an episomal vector, e.g., a nucleic acid capable of extra-chromosomal replication. Viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Viral vectors can be modified so the native tropism and pathogenicity of the virus are altered or removed. The genome of a virus also can be modified to increase its infectivity and to accommodate packaging of the nucleic acid encoding the polypeptide of interest.

Other non-limiting examples of vectors, suitable for use for the expression of high levels of recombinant proteins of interest include those selected from baculovirus, phage, plasmid, phagemid, cosmid, fosmid, bacterial artificial chromosome, viral DNA, Pl-based artificial chromosome, yeast plasmid, transposon, and yeast artificial chromosome. For example, the viral DNA vector is selected from vaccinia, adenovirus, foul pox virus, pseudorabies and a derivative of SV40. Suitable bacterial vectors for use in practice of the invention methods include pCG473, pCG512, pCG546, pCG550, pCG552, pJM1463, pJM1464, pQE70TM, pQE60TM, pQE-9TM, pBLUESCRIPTTM SK, pBLUESCRIPTTM KS, pTRC99aTM, pKK223-3TM, pDR540TM, PACTM and pRIT2TTM. Suitable eukaryotic vectors for use in practice of the invention methods include pWLNEOTM, pXTITM, pSG5TM, pSVK3TM, pBPVTM, pMSGTM, and pSVLSV40TM. Suitable eukaryotic vectors for use in practice of the invention methods include pWLNEOTM, pXTITM, pSG5TM, pSVK3TM, pBPVTM, pMSGTM, and pSVLSV40TM. One type of vector is a genomic integrated vector, or “integrated vector,” which is integrated into the chromosomal DNA of the host cell. Another type of vector is an episomal vector, e.g., a nucleic acid capable of extra-chromosomal replication. Viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Viral vectors are modified so that the native tropism and pathogenicity of the virus has been altered or removed. The genome of a virus also is modified to increase its infectivity and to accommodate packaging of the nucleic acid encoding the polypeptide of interest.

The nucleic acid construct (or the vector) of the present invention may be introduced into a host cell to be altered thus allowing expression of the protein within the cell. A variety of host cells are known in the art and suitable for proteins expression and extracellular vesicles production. Examples of typical cell used for transfection include, but are not limited to, a bacterial cell, a eukaryotic cell, a yeast cell, an insect cell, or a plant cell. For example, Human embryonic kidney 293 (HEK293), E. coli, Bacillus, Streptomyces, Pichia pastoris, Salmonella typhimurium, Drosophila S2, Spodoptera SJ9, CHO, COS (e.g., COS-7), 3T3-F442A, HeLa, HUVEC, HUAEC, NIH 3T3, Jurkat, FtetZ/CG473, FtetZ/293F, 293, 293F, F/YA24, 293H, HEK293, or 293F.

The nucleic acid construct of the present invention, included into a vector, may be introduced into a cell to be altered, thus allowing expression of the chimeric protein within the cell. A variety of methods are known in the art and are suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as LIPOFECTAMINE™, DOJINDO HILYMAX™, FUGENE™, JETPEI™, EFFECTENE™ and DREAMFECT™.

The terms “treat”, “therapeutic”, “prophylactic” and “prevent” are not intended to be absolute terms. Treatment, prevention and prophylaxis can refer to any delay in onset, amelioration of symptoms, improvement in patient survival, increase in survival time or rate, etc. Treatment, prevention, and prophylaxis is complete or partial. The term “prophylactic” means not only “prevent”, but also minimize illness and disease. For example, a “prophylactic” agent is administered to a subject, e.g., a human subject, to prevent infection, or to minimize the extent of illness and disease caused by such infection. The effect of treatment is compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques.

A treatment is considered “effective,” as used herein, if one or more of the signs or symptoms of a condition described herein are altered in a beneficial manner, other clinically accepted symptoms are improved, or even ameliorated, or a desired response is induced e.g., by at least 2%, 3%, 4%, 5%, 10%, or more, following treatment according to the methods described herein. Efficacy is assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (e.g., progression of the disease is halted). Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent is determined by assessing physical indicators of a condition or desired response. One skilled in the art can monitor efficacy of administration and/or treatment by measuring any one of such parameters, or any combination of parameters.

The term “effective amount” as used herein refers to the amount of a composition or an agent needed to alleviate at least one or more symptom of the disease or disorder and relates to a sufficient amount of therapeutic composition to provide the desired effect. The term “therapeutically effective amount” refers to an amount of a composition or therapeutic agent that is sufficient to provide a particular effect when administered to a typical subject. An effective amount as used herein, in various contexts, can include an amount sufficient to delay the development of a symptom of the disease, alter the course of a symptom disease (for example but not limited to, slowing the progression of a symptom of the disease), or reverse a symptom of the disease. For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control. The therapeutically effective amount may be administered in one or more doses of the therapeutic agent. The therapeutically effective amount may be administered in a single administration, or over a period of time in a plurality of doses.

“Immunogenically effective amount,” as used herein, means an amount of a composition sufficient to induce a desired immune effect or immune response in a subject in need thereof. In some embodiments, an immunogenically effective amount means an amount sufficient to induce an immune response in a subject in need thereof. In some embodiments, an immunogenically effective amount means an amount sufficient to produce immunity in a subject in need thereof, e.g., provide a protective effect against a disease such as viral infection. An immunogenically effective amount can vary depending upon a variety of factors, such as the physical condition of the subject, age, weight, health, etc.; the particular application, whether inducing immune response or providing protective immunity; the specific recombinant vector administered; the immunogen encoded by the recombinant vector administered; the specific antigenic polypeptide administered; and the particular disease, e.g., viral infection, for which immunity is desired. An immunogenically effective amount can readily be determined by one of ordinary skill in the art in view of the present disclosure.

“Administering” as used herein can include any suitable routes of administering a therapeutic agent or composition as disclosed herein. Suitable routes of administration include, without limitation, oral, parenteral, intravenous, intramuscular, subcutaneous, transdermal, airway (aerosol), pulmonary, cutaneous, injection or topical administration. Administration is local or systemic.

As used herein, the term “pharmaceutically acceptable” refers to a carrier that is compatible with the other ingredients of the formulation and not deleterious to the recipient thereof. The term is used synonymously with “physiologically acceptable” and “pharmacologically acceptable”. A pharmaceutical composition will generally comprise agents for buffering and preservation in storage, and can include buffers and carriers for appropriate delivery, depending on the route of administration. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “dose” and “dosage” are used interchangeably herein. A dose refers to the amount of active ingredient given to an individual at each administration. For the present invention, the dose can refer to the concentration of the extracellular vesicles or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable moiety (if present). One of skill in the art will recognize that the dose is modified depending on the above factors or based on therapeutic progress. The term “dosage form” refers to the particular format of the pharmaceutical, and depends on the route of administration. For example, a dosage form is in a liquid, e.g., a saline solution for injection. In the present invention, an illustrative dosage range of exosomes is from about 1×10⁶ to 1×10¹¹, about 6×10⁷ to 1×10¹¹, or for example 3×10¹⁰.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient is an individual that is seeking treatment, monitoring, adjustment or modification of an existing therapeutic regimen, etc.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The term “or” is, unless indicated otherwise, non-exclusive, i.e., encompassing both “and” and “or.” The term “any of between a modifier and a sequence means that the modifier modifies each member of the sequence. So, for example, the phrase “at least any of 1, 2 or 3” means “at least 1, at least 2 or at least 3”. The phrase “at least one” includes “a plurality”.

Definitions of common terms in cell biology and molecular biology is found in “The Merck Manual of Diagnosis and Therapy”, 19th Edition, published by Merck Research Laboratories, 2006 (ISBN 0-91 1910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

In some embodiments, the isolated polynucleotide comprises a sequence encoding a non-naturally occurring selectable marker (SM) protein, a degron domain (DD) and an exosome cargo (EC) protein. In some aspects, the SM protein is BsdR, BleoR, PuroR, HygR, NeoR. In some aspects, the DD is ER50 or ecDHFr, but it could be any appropriate destabilization domain. In some aspects, the EC protein is CD63. In other aspects, the EC protein is CD63/Y235A, but the EC protein could be any appropriate exosome cargo protein, such as CD63/Y235A, CD9, CD63, CD81, or any other suitable EC.

In some embodiments, the sequence encoding the DD and the sequence encoding the SM protein are separated by a sequence encoding a first linker domain, and the sequence encoding the DD and the sequence encoding the EC protein are separated by a sequence encoding a second linker domain. In some aspects, the second linker domain is cleavable or self-cleavable. In some aspects, the second linker is the self-cleavable viral 2a peptide. In other aspects, one or more of the coding sequences are operable linked to a regulatory control element which promotes expression of the coding sequences in a cell. The regulatory control element can be a CMV promoter.

In some embodiments, the amount of exosomes produced by the cell can increase by about 500%, about 600%, about 700%, about 800%, about 900%, or about 1000% or more as compared to a cell in the absence of the isolated polypeptide.

In other embodiments, the amount of EC protein within the exosomes is at least 20-fold higher, 30-fold higher, 40-fold higher, 50-fold higher, 60-fold higher, 70-fold higher, or 80-fold higher than in exosomes produced by a cell comprising the isolated polynucleotide lacking a sequence encoding a DD.

In various embodiments, the invention includes a method of producing exosomes. In some aspects, the method includes introducing an isolated polynucleotide into one or more cells in a first culture media, then selecting for cells which are resistant tozeocin, blasticidin, G418, puromycin, hygromycin or a combination thereof in a second culture media including zeocin, blasticidin, G418, puromycin, hygromycin, or a combination thereof. In further aspects. The method includes expanding the resistant cells in a third culture media, culturing the expanded population of resistant cells in a fourth culture media, and harvesting exosomes from the fourth culture media, thereby producing exosomes. The harvesting can be accomplished using methods well known in the art.

In another embodiment, the invention includes a pharmaceutical composition including an exosome produced by any of the methods described herein. The pharmaceutical composition can be used to treat viral or bacterial infections or to minimize the extent of illness and disease caused by such infection.

In some embodiments, the invention includes a method for producing extracellular vesicles (EVs) comprising: (i) inserting the coding region for an exosome cargo protein (EC) into an expression vector configured to drive recombinant EC expression; (ii) transfecting the expression vector into a cell line suitable for producing EVs; (iii) selecting and growing a transgenic cell line that expresses a high level of the recombinant EC in culture media; and (iv) collecting EVs from the conditioned tissue culture media. In some aspects, the EC is CD63/Y235A, CD9, CD63, CD81, or any other suitable EC. In another aspect, the high-level expression of the EC leads to approximately fourfold, fivefold, sixfold, sevenfold, eightfold, ninefold, or tenfold increase in the EV production yield.

In various embodiments, the method includes EVs, where the EVs are exosomes or microvesicles.

In some embodiments, the cell line suitable for producing EVs is human embryonic kidney 293 (HEK293), E. coli, Bacillus, Streptomyces, Pichia pastoris, Salmonella typhimurium, Drosophila S2, Spodoptera SJ9, CHO, COS (e.g., COS-7), 3T3-F442A, HeLa, HUVEC, HUAEC, NIH 3T3, Jurkat, FtetZ/CG473, FtetZ/293F, 293, 293F, F/YA24, 293H, HEK293, or 293Fin. In other aspects, the cell line suitable for producing EVs is a 293F-derived cell line.

In related embodiments, the 293F-derived cell line includes a transposon-containing vector which carries a transposon-carried gene in which the CMV enhancer/promoter is positioned to drive the expression of a bicistronic ORF encoding an AR protein, where the AR protein is an untagged, an ER50 degron-tagged, or an ecDHFR degron-tagged AR protein. For example, the AR protein is one or more of BsdR, ER50BsdR, ecDHFRBsdR, BleoR, ER50BleoR, ecDHFRBleoR, PuroR, ER50PuroR, ecDHFRPuroR, HygR, ER50HygR, ecDHFRHygR, NeoR, ER50NeoR, or ecDHFRNeoR, where the tag is indicated by the prefix ER50, ecDHFR, or by no prefix (untagged). In various aspects, the cell line suitable for producing EVs is F/YA22 or F/YA24. In a particular aspect, the cell line suitable for producing EVs is CD63/Y235A.

In some embodiments, the invention includes an expression vector for producing EVs, including the coding region for an EC. In some aspects, the expression vector includes a sequence encoding a non-naturally occurring selectable marker (SM) protein, a degron domain (DD) and an exosome cargo (EC) protein. In some aspects, the SM protein is BsdR, BleoR, PuroR, HygR, NeoR. In some aspects, the DD is ER50 or ecDHFr, but it could be any appropriate destabilization domain. In some aspects, the EC is CD63/Y235A, CD9, CD63, CD81, or any other suitable EC. In a particular aspect, the EC protein is CD63. In other aspects, the EC protein is CD63/Y235A, but the EC protein could be any appropriate exosome cargo protein.

Various embodiments provided herein relate to an isolated polynucleotide sequence encoding a modified protein expressed in fusion to TSPAN7/Y246A. These fusions are comprised of human TSPAN7 carrying a point mutation that inactivates its AP-2-binding site (Y246A) and modified peptides, proteins, or antigens expressed that may be appended to the N-terminus of TSPAN7/Y246A, the C-terminus of TSPAN7/Y246A, or inserted in the first or second extracellular loop of TSPAN7/Y246A. When fused to the N-terminus or C-terminus of TSPAN7/Y246A, these peptides, proteins and antigens will be located in the lumen of exosomes. When inserted in the first or second extracellular loop of TSPAN7/Y246A, these peptides, proteins and antigens will be located on the outer surface of exosomes.

In some embodiments, the present disclosure relates to an expression vector wherein the coding region includes from a 5′ to a 3′ end: a) a first inverted tandem repeat (ITR-1) flanking b) a region including: a selectable marker system (SMS), a promoter, an exosome cargo protein (EC), a woodchuck hepatitis virus post-transcriptional regulatory element (WPRE) and a polyadenylation site (pAn); c) a second ITR (ITR-r) d) a Rous sarcoma virus long-terminal repeat (RSV); e) a Sleeping Beauty transposase SB100x; and f) a polyadenylation site (pAn). In some aspects, the SMS encodes a polypeptide including a glutamine synthase (GS) protein, a porcine teschovirus 2a peptide linker and an antibiotic resistance (AR) protein. In additional aspects, the SMS further includes a promoter. In further aspects, the EC is a peptide, protein, or antigen expressed in fusion to TSPAN7/Y246A.

In some embodiments, the present invention relates to two empirically-derived pieces of information:
1) Determination of exosome cargo proteins (ECPs) that are highly efficient ECPs, defined as proteins that display the highest relative budding when expressed as recombinant proteins from introduced transgenes (relative budding=[amount of protein in exosomes]/[amount of protein in exosomes+amount of protein in cells]). Several such ECPs were identified by measuring the relative budding of numerous ECPs.
2) Determination of how to select for high and homogeneous expression of recombinant ECPs. This information was obtained by the work summarized in PCT/US2021/022200 published as WO/2021/183946, which describes an improved recombinant protein expression technology, including a new selectable marker gene (ER50BleoR) that was key to driving high-level expression of ECPs. The foregoing reference is incorporated by reference herein in its entirety.

The present disclosure relates to high-level expression of CD63/Y235A that leads to a 500% increase in exosome yield. Additionally, high-level expression of CD9 leads to a 1000% increase in exosome yield, and high-level expression of TSPAN7 leads to a 2000% increase in exosome yield. The exosomes produced by the corresponding cell lines are similar in size and shape to exosomes produced by normal control cells, as determined by (i) electron microscopic analysis of purified exosomes and (ii) nanoparticle tracking analysis (NTA).

This disclosure also provides a method of collecting exosomes from culture media. First, the coding region for an exosome carrier protein (ECP) is inserted into an expression vector that is designed to drive the very highest level of recombinant ECP expression. Next, the vector is transfected into the desired exosome-producing cell line. Then, the transgenic cell lines that express the very highest level of the recombinant ECP are selected. Finally, the transgenic line that expresses the highest level of the recombinant ECP is cultured in culture media, and exosomes are collected from the culture media. Cell lines recommended for these methods include 293F-derived cell lines that produce exosomes at higher yield than the parental cell lines. The increased yield can be 5-fold, 10-fold, 20-fold, or more.

### EXAMPLES

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### Material and Methods

### Plasmids

Plasmids used in this study were based on the Sleeping Beauty transposon-carrying vector pITRSB. Each carries a single gene designed to express a polycistronic ORF in which (i) the coding sequence of the protein of interest is followed by (ii) codons for the porcine teschovirus 2a peptide and then (iii) the coding sequence of the antibiotic-resistance protein, either untagged or carrying an N-terminal destabilization domain. Plasmid maps were assembled using SnapGene software, all coding sequences were confirmed by DNA sequence analysis, and plasmids were prepared using commercial alkaline lysis and purification kits (Promega). Plasmids were maintained and amplified in DH10B Escherichia coli cells grown in ampicillin-containing Luria broth media.

### Cell Culture

293F cells (catalog no.: A14528; Thermo Fisher Scientific) were grown in complete media (chemically defined media [CM], Dulbecco's modified Eagle's medium supplemented with 10% fetal bovine serum and 1% penicillin/streptomycin) at 37 C and 5% CO₂. For stable clone selection, 293F cells were transfected with plasmid DNAs using Lipofectamine 3000 (Thermo Fisher Scientific), incubated for 48 h in CM, and then split into selective media (CM containing the appropriate amount of cognate antibiotics [400 μg/ml G418, 20 μg/ml blasticidin, 400 μg/ml hygromycin B, 3 μg/ml puromycin, or 200 μg/ml zeocin]). Antibiotic-resistant clones were fed every 3 to 4 days in selective media until distinct drug-resistant clones were large enough to be seen by eye, typically 2 weeks. The thousands of antibioticresistant SCCs that arose from each transfection were then pooled to create a single polyclonal cell line from each transfection, which were then expanded for an additional 2 weeks under antibiotic selection. Cells were then processed for flow cytometry, fluorescence microscopy, exosome collection, and immunoblot.

### Flow Cytometry

For measurement of mCherry fluorescence, cells were washed in Hank's buffered saline solution (HBSS; Thermo Fisher Scientific), released from tissue culture plates using trypsin/EDTA solution, and resuspended at a concentration of 1×107 cells per ml in HBSS containing 0.10% fetal bovine serum at 4° C. Cell suspensions were maintained on ice, diluted to a concentration of 1×106 cells per ml, and examined for mCherry fluorescence by flow cytometry on a Guava easyCyte flow cytometer (Luminex) set to the appropriate excitation and detection wavelengths. The relative brightness was determined for thousands of individual cells in each cell line using InCyte software (Luminex) and replotted with FlowJo (Beckton Dickinson), version 10, as scatter plots, average relative brightness, and CV.

For measurement of surface CD63 abundance and Cy5-CP05 peptide binding, cells were washed in HBSS, released from tissue culture plates using trypsin/EDTA solution, and resuspended at a concentration of 1×107 cells per ml in HBSS. Cell suspensions were maintained on ice, diluted to a concentration of 1×106 cells per ml, incubated with either (i) fluorescently labeled anti-CD63 antibody (catalog no.: 353006; BioLegend) or (ii) CY5-labeled CP05 peptide (NH2-CRHSQMTVTSRL(K/Cy5)-amide; Vivitide; molecular weight=11,910). Following the binding reaction, cells were washed five times in PBS and then examined by flow cytometry on a Beckman CytoFlex flow cytometer set at the appropriate excitation and emission wavelengths. The relative brightness was determined for thousands of individual cells in each cell line using Beckman CytExpert software (version 2.3.1.22) to calculate average brightness and CV as well as to generate scatter plots and histograms.

### Microscopy

Cells were grown overnight on poly-L-lysine-coated cover glasses. Cells were then washed in PBS and fixed in 3.7% formaldehyde in PBS for 30 minutes. Cover glasses were then placed cells-side-down on 0.050 mL of FITC-labeled anti-CD63 antibody, diluted in PBS, for 30 minutes (catalog #353006, Biolegend), washed 5 times in PBS, and then fixed to glass slides using 0.007 mL of Fluoromount-G (Electron Microscopy Sciences).

### Exosome Purification

293F cells and derivative clones were seeded into FreeStyle 293 Expression Medium (catalog no.: 12338-018; Gibco) at a density of 1.5×10⁶ cells/ml in shaker flasks in a volume of approximately one-fourth the flask volume and grown for 3 days at a shaking speed of 110 rpm. Cells and large cell debris were removed by centrifugation at 5000 g for 15 min. The supernatant was passed through an 200 nm pore size diameter sterile vacuum filtration unit (SteriFlip; Millipore/Sigma) to yield a clarified tissue culture supernatant. The clarified tissue culture supernatant was then subjected to two 30 min-long spins at 10,000 g to remove any contaminating microvesicles, followed by collection of exosomes by spinning the samples at 100,000 g for 2 h, discarding the supernatant, and resuspending the exosomes in PBS.

### Immunoblot of Cell and Exosome Fractions

Cells and exosomes were lysed in SDS-PAGE sample buffer, separated by SDS-PAGE, transferred to polyvinylidene difluoride membranes, and blocked for 1 h in blocking solution (5% nonfat milk in TBST (50 mM Tris-HCl, pH 8.0, 138 mM NaCl, 2.7 mM KCl, 0.05% Tween-20). The blocked membranes were then incubated overnight with primary antibody diluted into blocking solution at 4° C., with gentle rocking. Following extensive washes in TBST, the polyvinylidene difluoride membranes were incubated with horseradish peroxidase (HRP)-conjugated secondary antibodies, again in blocking solution. The membranes were again washed extensively in TBST, followed by chemiluminescent detection of HRP using ECL Plus detection reagents (GE Healthcare). Antibodies were obtained from commercial sources (mouse monoclonal anti-CD63 antibody [catalog no.: NBP2-32830] was from NOVUS, anti-heat shock protein 90 antibody [catalog no.: sc-13119] was from Santa Cruz Biotechnology, and secondary HRP-conjugated antibodies were from Jackson ImmunoResearch).

### Creation and Validation of CD63-Deficient 293F Cells

293F cells were transfected with purified Cas9 protein (catalog no.: A36498; Invitrogen) and a gRNA (Invitrogen) targeting the sequence 5′-AACGAGAAGG CGATCCATA[AGG]-3′ (SEQ ID NO: 1) in exon 5 of the CD63 gene. Following 3 days of incubation, CD63-deficient cells were isolated by fluorescence activated cell sorting (FACS) using an FITC-conjugated anti-CD63 antibody (catalog no.: NBP2-32830, NOVUS) on a Sony SH800S FACS machine. Single cells were seeded into a 96-well plate and expanded until the media began to turn acidic. To identify the CD63 mutations in different single cell clones (SCCs), genomic DNA was extracted using DNeasy Blood & Tissue Kit (Qiagen), amplified by PCR using locus-specific primers, subcloned into a plasmid vector, expanded as single clones in bacteria, and then 6 individual clones were sequenced from plasmids derived from each genomic DNA PCR. To measure CD63 mRNA abundance, RNA was extracted by Quick-RNA Microprep Kit (Zymo Research), followed by reverse transcription with High-Capacity RNA-to-cDNA Kit (Applied Biosystems) and quantitative real-time PCR using SYBR Green qPCR Master Mix (Bio-Rad), gene-specific primers for CD63 mRNA (5′-CAGTGGTCATCATCGCAGTG-3′ (SEQ ID NO: 4) and 5′-ATCGAAGCAGTGTGGTTGTTT-3′ (SEQ ID NO: 5)) and 18S rRNA (5′-CGGCGACGACCCATTCGAAC-3′ (SEQ ID NO: 6) and 5′-GAATCGAACCC TGATTCCCCGTC-3′ (SEQ ID NO: 7)), and the CFX96 Real-Time PCR detection system (Bio-Rad). The expression of CD63 mRNA relative to 18S ribosomal RNA was calculated using the AACT method.

### Statistical Analysis

Statistical analysis was performed using GraphPad Prism 8 software for Windows/Mac (GraphPad Software, La Jolla California USA) or Excel (Microsoft). Flow data are reported as mean, median and coefficient of variation, while immunoblot data is reported as mean±standard error, with significance determined by one-way analysis of variance.

### Example 2

### Degron Tagging of AR Proteins

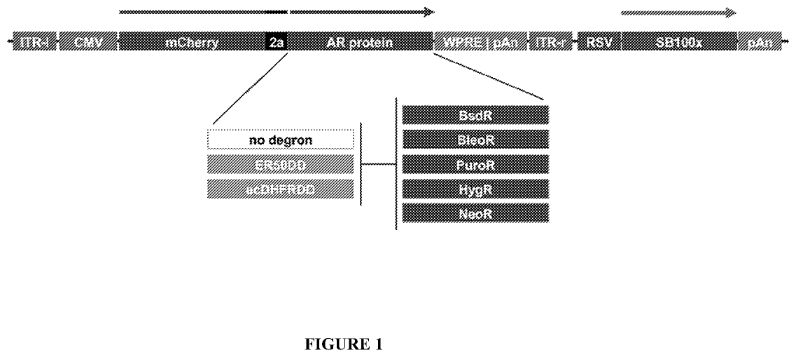

To determine whether destabilization domains could impact the levels of transgene expression, pITRSB-based Sleeping Beauty transposons were generated (Guo et al., 2021; Ochmann and Ivics, 2021) that use CMV enhancer/promoter sequences to drive the expression of bicistronic open reading frames (ORFs) (FIG. 1). These ORFs encode (i) the fluorescent protein mCherry (Shaner et al., 2004), (ii) an 18 amino acid-long viral 2a peptide (p2a) (Kjaer and Belsham, 2018; Luke et al., 2010), and (iii) one of 15 different AR proteins, which correspond to untagged, ER50-tagged, or ecDHFR-tagged forms of the BsdR, NeoR, HygR, PuroR, and BleoR proteins. These 15 transposon-carrying vectors were transfected into 293F cells, followed two days later by transfer of the transfected cell populations into selective media. Selective media were changed every 3 to 5 days for 2 weeks, resulting in the death of all untransfected cells, as well as 11 transgenic cell lines that failed to express enough of the AR protein to confer survival and growth in the antibiotic-containing media. At the end of this period, thousands of single-cell clones (SCCs) were apparent, which together represent the range of transgene expression levels generated by each vector. To capture this range, all SCCs from each transfection were pooled to create a single polyclonal cell line. SCCs were generated from all transfections except for one (cells transfected with the ecDHFRBleoR-based vector yielded only a very small number of poorly adherent and poorly growing cells, and the resulting line was unsuitable for further analysis).

The 14 polyclonal cell lines that emerged from this process were expanded for 2 additional weeks in selective media and then interrogated by flow cytometry to measure the mean, median, and individual mCherry expression levels across 20,000 cells from each cell line. Degron-tagged forms of the BsdR, BleoR, PuroR, HygR, and NeoR proteins were selected for higher levels of expression of the linked recombinant protein mCherry (Table 1). However, the results varied significantly between these different AR genes, warranting a more in-depth discussion of each group of antibiotic-resistant cell lines.

TABLE 1
Flow cytometry measurement mCherry fluorescence brightness
(a.u.) of transgenic 293F-derived cell lines

| | | Increase | | Increase | Mean/ | |
|---|---|---|---|---|---|---|
| AR Protein | Mean | in Mean | Median | in | Median | CV |
| BsdR | 1308 | n.a. | 28 | n.a. | 47 | 370 |
| ER50BsdR | 6646 | 5.1x | 3424 | 122x | 2.0 | 140 |
| ecDHFRBsdR | 7978 | 6.1x | 4696 | 168x | 1.7 | 136 |
| BleoR | 16025 | n.a. | 13093 | n.a. | 1.2 | 84 |
| ER50BleoR | 37141 | 2.3x | 33678 | 2.6x | 1.1 | 48 |
| ecDHFRBleoR | n.d. | n.d. | n.d. | n.d. | n.d. | n.d. |
| PuroR | 6539 | n.a. | 4595 | n.a. | 1.4 | 107 |
| ER50PuroR | 10808 | 1.7x | 8592 | 1.9x | 1.3 | 77 |
| ecDHFRPuroR | 10898 | 1.7x | 8780 | 1.9x | 1.2 | 81 |
| HygR | 6807 | n.a. | 3912 | n.a. | 1.7 | 125 |
| ER50HygR | 6532 | 0.9x | 4567 | 1.2x | 1.4 | 97 |
| ecDHFRHygR | 8455 | 1.2x | 5961 | 1.5x | 1.4 | 99 |
| NeoR | 4498 | n.a. | 2543 | n.a. | 1.8 | 126 |
| ER50NeoR | 5748 | 1.3x | 3828 | 1.5x | 1.5 | 110 |
| ecDHFRNeoR | 5790 | 1.3x | 3852 | 1.5x | 1.5 | 107 |

### Example 3

### Debron Tagging Improved Blasticidin/Bsdr-Selected Transgene Expression by 6-Fold

Of the five antibiotics commonly used in mammalian cell transgenesis experiments (blasticidin, geneticin, hygromycin B, puromycin, and zeocin), blasticidin kills fastest of all, as addition of blasticidin killed 100% of 293F cells within 48 h. Curiously, when 293F cells were transfected with the transposon carrying the CMV-mCherry-2a-BsdR transgene, relatively little cell death was observed, and nearly all cells in the population exhibited robust growth. These results indicate that most of the cells (i) had been transfected, (ii) continued to express the BsdR protein, and (iii) expressed enough blasticidin deaminase enzyme activity to confer blasticidin resistance on the cells, regardless of the level of transgene expression.

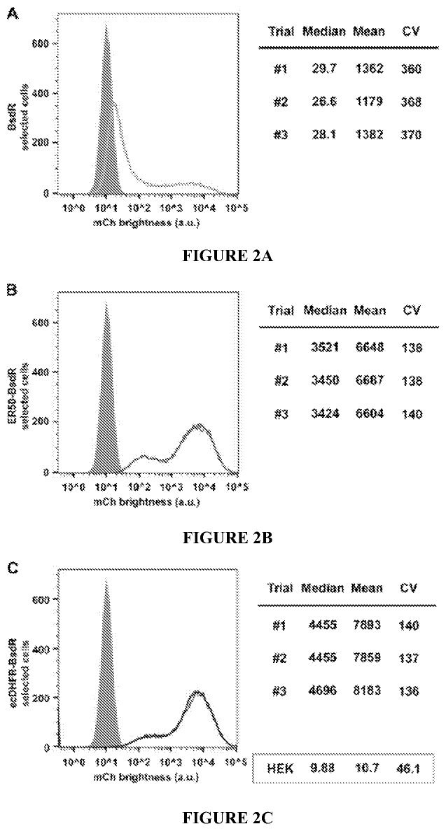

Consistent with this notion, the blasticidin-resistant cell line resulting from transfection with this BsdR-based vector displayed the lowest level of mCherry expression and the greatest cell-to-cell heterogeneity in mCherry of any cell line tested (Table 1 and FIG. 2A). Many of the blasticidin-resistant cells in this polyclonal cell line displayed levels of mCherry fluorescence that were no higher than the background fluorescence of 293F control cells. In contrast to the BsdR-selected cell line, the ER50BsdR-selected cell line displayed mCherry expression levels that were, on average, approximately fivefold higher than the BsdR-selected cell line, while the ecDHFRBsdR-selected cell line displayed average mCherry expression approximately six-fold higher than the BsdR-selected cell line (Table 1, FIG. 2B, FIG. 2C). The pronounced effect of BsdR degron tagging on transgene expression is evident from a number of perspectives, which include (i) 122-fold higher and 168-fold higher median fluorescence levels, (ii) the threefold lower CV values (365), and (iii) the drop in mean/median ratio from 45 to 2.

### Example 4

### ER50BleoR, the Most Restrictive Dominant Selectable Marker

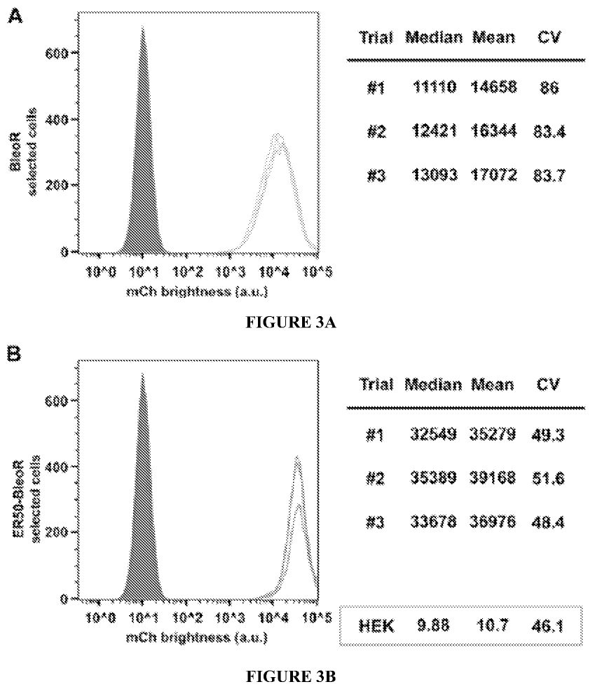

The BleoR gene selects for higher and more homogeneous transgene expression than the BsdR, PuroR, HygR or NeoR selectable marker genes (Guo et al., 2021). The same was observed in the present study, as the mean mCherry fluorescence of the BleoR-selected cell line was >10-fold higher than that of the BsdR-selected cell line (Table 1). In perhaps the most important invention of this study, a degron-tagged form of BleoR, ER50BleoR, exhibited a further 2.3-fold increase in average mCherry expression relative to the BleoR-selected cell line (Table 1; FIG. 3A, FIG. 3B).

This 2.3-fold increase in mean transgene expression led to a further reduction in the cell-to-cell heterogeneity of mCherry expression, shown here by the drop in the coefficient of variation from 84 to 48. It should, however, be noted that >99% of the cells in both the BleoR and ER50BleoR-selected cell line expressed mCherry fluorescence that was >1000-fold above background, evidence that both forms of the BleoR gene are quite good at generating cell lines in which nearly every surviving cell expresses a high level of transgene expression. That being said, the superior performance characteristics of the ER50BleoR marker can be seen throughout these data, even in the mean/median ratio that fell to 1.1, just shy of the theoretical optimum of 1. Taken together, these results demonstrate that the ER50BleoR AR gene can be used to rapidly select for polyclonal cell lines that express the very highest levels of linked transgene expression, without the need for isolating and screening large numbers of SCCs. However, if SCCs are needed, selection with the ER50BleoR marker is likely to eliminate all but the highest expressing clones, reducing the number of clones that need to be screened to identify the highest-expressing cell lines with high and homogeneous expression of a protein of interest, without the need for isolating and screening large numbers of single cell clones (SCCs).

### Example 5

### Degron Tagging Increased PuroR-Selected Transgene Expression by 70%

PuroR and HygR genes yield the second-highest levels of linked recombinant protein expression, about 50% that of zeocin-resistant BleoR-derived cell lines, yet significantly higher than cell lines created using the BsdR or NeoR selectable marker genes (Guo et al., 2021). In the present study, PuroR- and HygR-based cell lines expressed nearly identical levels of mCherry fluorescence (6539 versus 6807 arbitrary units [a.u.], respectively), a level that was ˜40% the level selected by the BleoR marker (16,025 a.u.), and higher than the levels of transgene expression observed in cell lines selected via the NeoR (4498 a.u.) or BsdR (1308 a.u.) markers (Table 1).

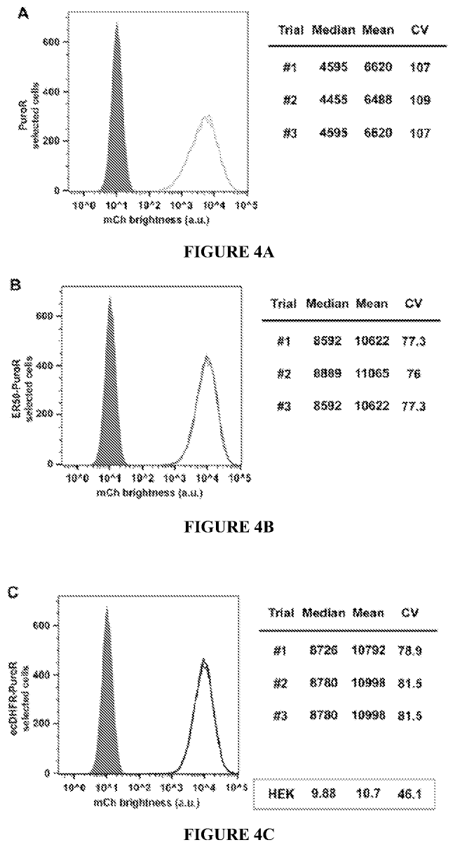

As for degron-tagged forms of PuroR, they selected for ˜70% higher mean mCherry expression (FIG. 4A-FIG. 4C). While the amplitude of this effect was less than observed for the BsdR and BleoR AR genes, it did lead to reduced heterogeneity of expression, shown here by a drop in CV from 107 to ˜80, and a fall in mean/median ration from 1.4 to 1.2.

### Example 6

### Deron Tagging had Only Minimal Effects on HyR- and NeoR-Selected Transgene Expression

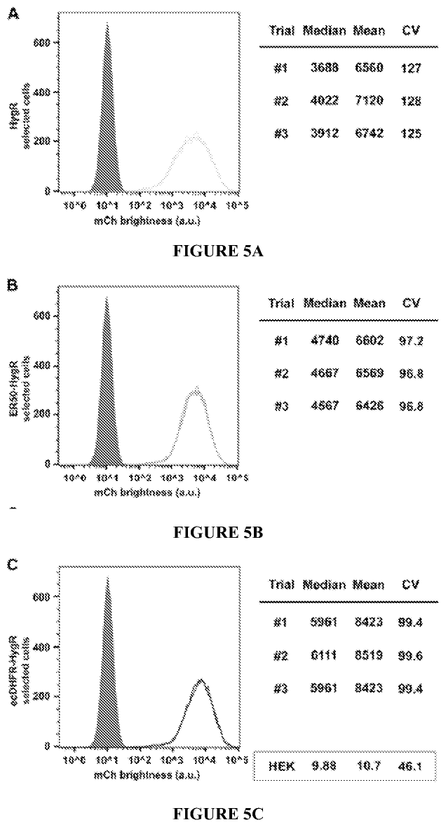

In contrast to BsdR, BleoR, and PuroR, HygR HygR was at most slightly sensitive to degron tagging. While both degron-tagged forms of the HygR marker resulted in slight increases in the homogeneity of expression, the increase in mean mCherry expression was either undetectable (in the case of ER50HygR-selected cells) or only 24% (in the case of ecDHFRHygR-selected cells) (FIG. 5A-FIG. 5C). This was unexpected, and raised questions about why degron tagging could have a strong effect on some AR proteins but little if any effect on others.

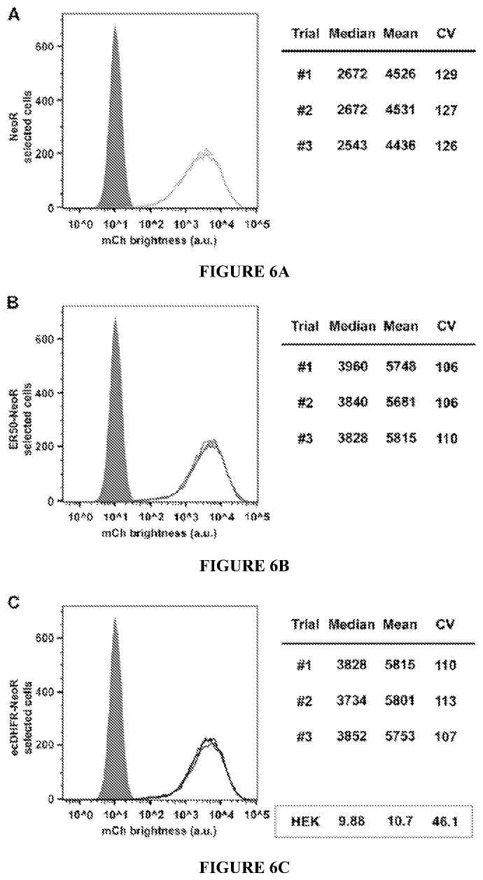

A similar question arose from analysis of the geneticin resistant cell lines (FIG. 6A-FIG. 6C). The NeoR-selected cell line displayed mCherry levels that were lower and more homogeneous than those selected by the BleoR, PuroR, and HygR markers, and higher only than the BsdR-selected cells (Table 1). However, degron tagging had only a minimal effect on mean mCherry expression, increasing it by only 30% and and effecting only a slight drop in CV values from 126 to about 110.

### Example 7

### ER50BleoR-Based Selection has a Preferential Effect on Exosome Engineering

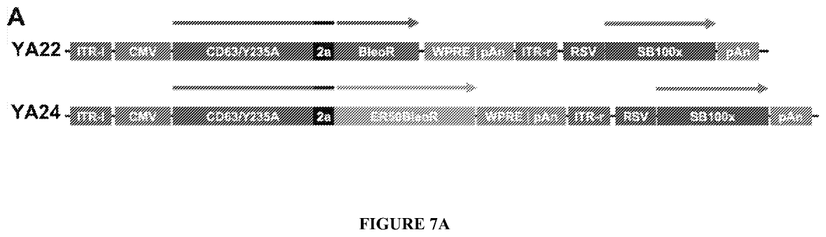

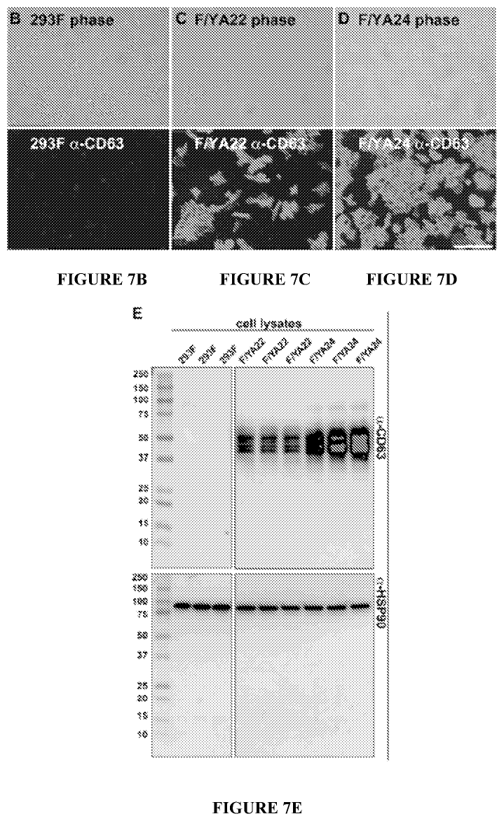

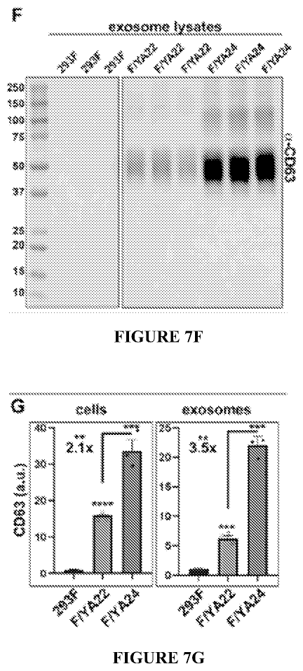

The primary question arising from these studies is whether use of the ER50BleoR marker dives improved cell engineering, and more specifically, an improvement in exosome engineering. Towards this end, a pair of Sleeping Beauty transposon vectors were designed to drive the chromosomal integration of a single transgene that expresses a bicistronic ORF encoding (i) CD63/Y235A (a mutant form CD63 that displays 6-fold higher budding than WT CD63 (Fordjour and Gould, 2019)); (ii) a viral 2a peptide (Luke et al., 2010); and (iii) either the BleoR or ER50BleoR proteins (FIG. 7A). These vectors were transfected into 293F cells, which were then incubated in nonselective media for two days, followed by addition of zeocin to select for transgenic cell lines. Two weeks later, all zeocin-resistant clones from each transfection were pooled, creating the cell lines F/YA22 (BleoR-based) and F/YA24 (ER50BleoR-based). These cell lines were expanded under selection for an additional two weeks and then examined for the expression and vesicular secretion of CD63.

Immunofluorescence microscopy confirmed that the F/YA22 and F/YA24 cell lines did indeed express far more CD63 than the parental 293F cell line (FIG. 7B-FIG. 7D). As for the magnitude of this increase, immunoblot analysis of cell and exosome fractions collected from all three cell lines (FIG. 7E-FIG. 7G) revealed that F/YA22 cells contained ˜15-fold more CD63 (n=3; p=0.00000013), whereas F/YA24 cells contained ˜30-fold more CD63 relative to 293F cells (n=3; p=0.00044) (FIG. 7E and FIG. 7G). The 2.1-fold higher level of CD63 expression seen for the ER50BleoR-selected cell line (n=3, p=0.0045) is consonant with the 2.3-fold increase observed for mCherry expression, indicating that the ER50BleoR drives higher recombinant protein expression, even for proteins as dissimilar as the soluble cytoplasmic mCherry and the polytopic membrane protein CD63/Y235A.

In addition to its utility for cell engineering, these data indicate that this marker may be of even greater utility for exosome engineering. Specifically, F/YA22-derived exosomes contained sixfold more CD63 than 293F-derived exosomes (n=3; p=0.00038) but F/YA24-derived exosomes contained 22-fold more CD63 than 293F cells (n=3; p=0.00051). This 3.5-fold increase in the loading of an exosomal cargo protein (n=3; p=0.0005) raises the intriguing possibility that exosome cargo protein loading operates preferentially at the very highest levels of exosome cargo protein expression.

### Example 8

### Cy5-Labeled CP05 Peptide Binds 293F Cells in a CD63-Independent Manner

It has been reported that a short peptide, CP05 (NH2-Cys-Arg-His-Ser-Gln-Met-Thr-Val-Thr-Ser-Arg-Leu-COOH, SEQ ID NO: 8), binds specifically to the extracellular domain of CD63, and moreover, that this peptide can be used to dock a diverse array of other peptides and oligonucleotides on the surface of exosomes, imbuing them with specific biological activities and tissue tropisms (Gao et al., 2018). These reports raise the interesting possibility that the exosomes released by F/YA24 cells, which contain 20-fold more CD63, might be the ideal type of exosomes for CP05-based exosome engineering.

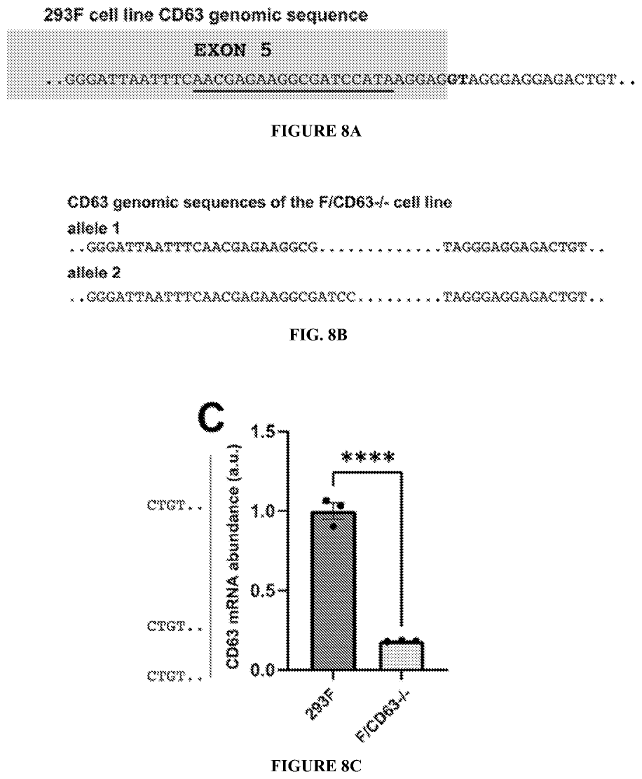

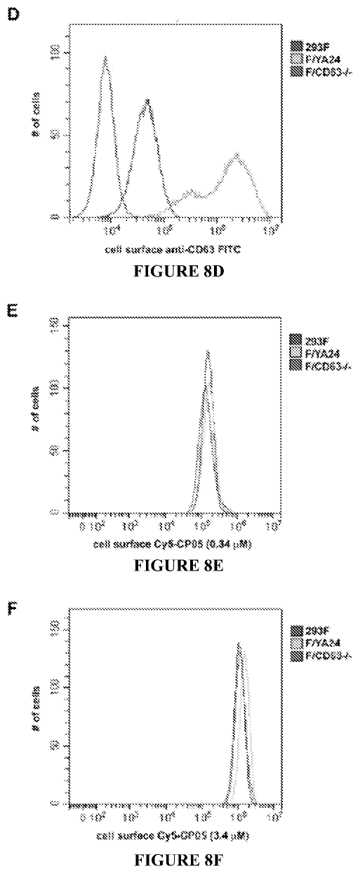

In anticipation of using CP05 peptides to engineer exosomes, a CD63^{-/-} cell line was envisioned that could be used as a negative control for CP05-based engineering studies. Toward this end, 293F cells were transfected with recombinant Cas9 protein mixed with a CD63-specific guide RNA (gRNA) that targets the 3′ end of fifth common exon of the CD63 gene (5′-AACGAGAAGGCGATCCATAAGG-3′, SEQ ID NO: 1 nucleotides 13-31; protospacer adjacent motif site is given in bold; FIG. 8A). The transfected cells were grown for several days (to allow for CD63-deficient cells to allow for turnover of pre-existing CD63 mRNA and protein) and then processed for fluorescence-activated cell sorting (FACS) using a fluorescently tagged anti-CD63 antibody, with individual CD63-deficient cells sorted into distinct wells of a 96-well plate. After 2 weeks of culture, numerous SCCs were interrogated by quantitative RT-PCR to identify cell lines that had a significant reduction in CD63 mRNA abundance, as many of the most severe mutations in human genes (e.g., spice site, nonsense, and frameshift mutations) result in mRNA destruction by the nonsense-mediated RNA decay machinery. Candidate CD63^{-/-} cell lines were then interrogated by sequence analysis of genomic DNA in the vicinity of the Cas9/gRNA cut site. The 293F/CD63^{-/-} cell line used in the remainder of this study has two mutant CD63 alleles, both of which had deletions that removed the splice donor site at the 5′ end of intron 5 (FIG. 8B). These mutations preclude the proper splicing of the CD63 gene and appear to have induced nonsense-mediated RNA decay turnover of their cognate mRNAs, as seen in the approximately sevenfold reduction in total CD63 RNA abundance observed for this cell line (FIG. 8C). These data cannot, however, exclude the possibility that a small amount of C-terminally truncated CD63 proteins might be expressed in this cell line, though these CD63 proteins would lack its C-terminal 54 amino acids, including the last 18 amino acids of the second extracellular loop and the entirety of the fourth transmembrane domain and cytoplasmic tail.

To determine whether these mutations led to a drop in the cell surface expression of CD63, cell lines were interrogated: the 293F/CD63^{-/-} cell line, the parental 293F cell line, and the F/YA24 cell line by flow cytometry, using a commercially available fluorescently tagged monoclonal anti-CD63 antibody. The average staining intensity for the 293F/CD63^{-/-} cell line (FIG. 8C, purple line) was approximately fivefold lower than that of the WT 293F cell line (FIG. 8C, red line) and 200-fold lower than that of the F/YA24 cell line, which expresses high levels of CD63/Y235A (FIG. 8C, green line). Thus, while it is possible that CD63^{-/-} cells express some small amount of a truncated CD63 protein, they clearly display less cell surface CD63 than these other cell lines, even though the preferred control of a matched isotype staining was not included in these experiments. With these three cell lines in hand, the foundation of the planned CP05-based exosome engineering platform was tested by investigating whether a fluorescently tagged CP05 peptide would bind the surface of these cell lines in a CD63-dependent manner. Specifically, the peptide NH2-CRHSQMTVTSRL(K/Cy5)-amide (SEQ ID NO: 8) was synthesized, resuspended in cell-labeling buffer, incubated with all three cell lines, the cells were washed, and then each was interrogated by flow cytometry. Surprisingly, this peptide displayed strong binding to the surface of all three cell lines, evident here in the strong Cy5 fluorescence detected for all three cell populations (FIG. 8D). Furthermore, these experiments were repeated using a 10-fold higher concentration of CP05, a 10-fold increase in the cell surface labeling of all three cell lines was observed (FIG. 8E). Taken together, these results are consistent with prior observations showing that the CP05 peptide binds biological membranes but provide additional clarity to those results by showing that CP05 binds to cell membranes in a CD63-independent manner.

### Example 9

### Discussion

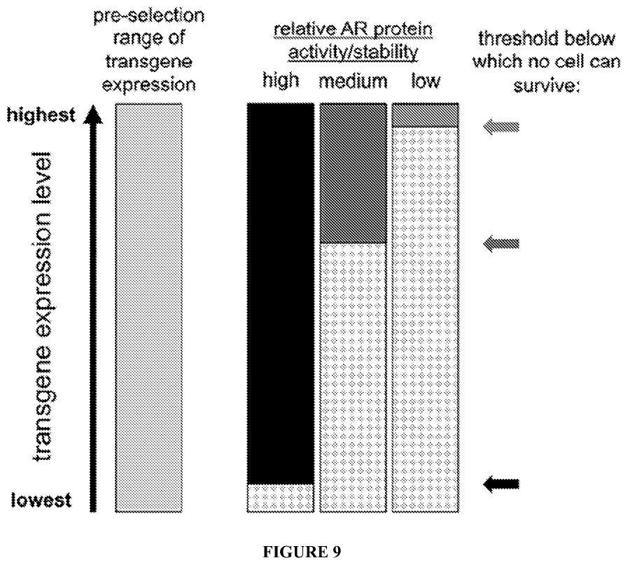

Choice of AR gene is a major factor in determining the level of transgene expression in antibiotic-resistant cell lines. The data presented in the present study confirm prior observations while also providing new empirical support for the operating hypothesis. Specifically, it appears that use of the shortest-lived and least-active AR proteins yield cell lines with the highest and most homogeneous levels of transgene expression (FIG. 9), thus explaining why AR proteins fused proteasome-targeting degrons selected for higher levels of linked transgene expression. Furthermore, this was observed for two of the most well-characterized degrons, the ER50 and ecDHFR destabilization domains, which have the added feature of being conditionally stabilized by small molecules (4-hydroxytamoxifen and trimethoprim, respectively), raising the possibility that transgene expression might be further tuned by carrying out antibiotic selection in the presence of different concentrations of these drugs.

While the degron-tagging approach was generally successful, the magnitude of the degron-induced increase in transgene expression varied dramatically, from a high of the 610% increase observed for degron-tagged BsdR, to the low of the 24% increase observed for degron-tagged HygR. It is not known why different AR proteins display differential sensitivity to degron tagging. However, several factors indicate that reaction mechanism may be a contributing factor. For example, BleoR is the only AR protein that is not an enzyme, does not act catalytically, and instead inactivates its cognate antibiotics (i.e., zeocin) by chelating them in complexes with a 1:1 stoichiometry. Given this nonenzymatic stoichiometric mode of action, degron tagging of the BleoR protein is predicted to have a relatively direct effect on linked transgene expression, and this is borne out by the fact that the ER50BleoR selected for approximately twofold higher levels in the linked expression of two unrelated recombinant proteins, mCherry and CD63/Y235A.

As for the four other AR proteins, they all encode an enzyme of one activity or another but differ rather dramatically in their requirement for essential cosubstrates, and in the nature of these cosubstrates. For example, BsdR encodes a blasticidin deaminase that acts by a simple hydrolysis reaction mechanism, with no known binding to any cosubstrate or cofactor present in the cell. This may render the BsdR protein particularly sensitive to degron tagging, as there is no other factor available to stabilize the protein against proteasomal degradation. On the opposite end of the scale are the HygR and NeoR proteins, both of which bind ATP because of their phosphotransferase reaction mechanisms. Without any data, it is pure speculation to suggest this, but it is at least formally possible that the presence of ATP may stabilize these proteins against degron tagging-induced turnover, much as small molecules bound by the ER50 and ecDHFR proteins stabilize them against proteasomal turnover. A similar possibility exists for the puromycin acetyltransferase encoded by PuroR, which uses a different metabolite as cosubstrate, acetyl-CoA.

Regardless of why degron tagging has more pronounced effects on some markers than on others, these data clearly show that a degron-tagged BleoR gene selected for higher levels of linked transgene expression than any of the other AR genes tested, untagged or degron tagged. As for whether other AR genes can be developed that select for even higher levels of linked recombinant protein expression, it is an open question. After all, the operating hypothesis is that the choice of AR gene has no effect on the intrinsic levels or range of transgene expression, but rather, is made manifest by the killing of cells that do not express enough of the transgene to render cells resistant to the antibiotic (FIG. 9). This hypothesis predicts that there is a finite limit to the extent to which AR gene engineering will improve the expression of linked transgenes. In fact, the data suggest that this limit is already being approached in the case of the BleoR marker, as one degron tagged form, ER50BleoR, selected for a twofold increase in average expression whereas tagging BleoR with the more restrictive degron, ecDHFR, failed to yield any rapidly growing colonies. If this hypothesis is correct, the ER50BleoR marker may represent a starting point for identifying and optimizing the other variables that limit the expression of recombinant proteins.

While ER50BleoR may be approaching the limit to which the BleoR protein can be improved, these data indicate that there is still significant room for improvement of the four other AR genes. Specifically, there appears to be no reason why these selection systems cannot be improved to at least the level attained by ER50BleoR (Table 1). This suggests that there should be some way to improve puromycin selection by 3.4-fold higher than ecDHFRPuroR, improve hygromycin selection ˜4.4-fold more than ecDHFRHyg, improve blasticidin selection by ˜4.7-fold more than ecDHFRBsdR, and improve geneticin/G418 selection by ˜6.4-fold higher than ecDHFRNeoR.

### ER50BleoR-Mediated Exosome Engineering

The present invention was based on the question of whether the improved ER50BleoR marker might have an even stronger effect on exosome engineering. The data indicate that it does, as the approximately two-fold higher level of CD63/Y235A expression led to an even larger 3.5-fold increase in the exosomal secretion of CD63/Y235A.

### CD63-Independent Binding of Cell Membranes by CP05-Cy5

CD63 is an exosome marker protein. To identify a CD63-specific ligand, and therefore a way to selectively label exosomes, Gao et al. (2018) carried out a phage display screen using the purified second extracellular loop of CD63 as bait. This screen yielded a short peptide (NH2-CRHSQMTVTSRL-COOH) as a candidate CD63-binding ligand. Subsequently, this peptide was used for a technique known as “exosome painting” in which CP05 peptides were used to noncovalently attach a variety of functional and tropism-altering macromolecules to the outer surface of exosomes. In the context of these results, development of cells and exosomes that display significantly higher levels of CD63 seemed a logical partner to the CP05 technology; however, in analysis of the articles published on the CP05 peptide, there was no data on the affinity of the CP05 peptide for recombinant CD63 protein. There was no direct evidence of CP05-CD63 interaction of any kind. In addition, there was no evidence that the CP05 peptide requires the presence of CD63 for its membrane-binding activity.

In light of these considerations, the observation that the CP05 peptide bound equally well to F/CD63^{-/-} cells, to WT 293F cells, and to 293F cells that express 40-fold higher levels of surface CD63 is not in conflict with any prior published data. It is, however, in stark conflict with the hypothesis that CP05 binds to exosome membranes in a CD63-dependent manner. While it is formally possible that some unknown variable causes nonspecific binding of the CP05 peptide used in this study, the simplest explanation for the results is that CP05 peptides bind biological membranes in a nonspecific fashion. Furthermore, nonspecific binding of the CP05 peptide to the surface of biological membranes is entirely consistent with its chemical structure (CRHSQMTVTSRL, SEQ ID NO: 8), which in just 12 amino acids displays three positively charged side chains (R2, H3, and R11), four hydroxylated side chains (S4, T7, T9, and S10), and two polar side chains (C1 and Q5). In short, the CP05 peptide is a highly polar polycationic peptide nearly ideally suited for nonspecific binding to biological membranes, which are known to have a nearly unsaturable capacity for binding polar polycationic polymers. In conclusion, the CP05 peptide may be a way to “paint” the exosome surface with CP05-coupled molecules.

### Example 10

The high clinical potential of exosomes remains largely unrealized. A new model of exosome biogenesis proposes that exosome biogenesis is mediated by a shared, stochastic mechanism that operates along a spectrum of plasma and endosome membrane, that most exosomes and exosome cargo proteins bud directly from the plasma membrane, and that exosome biogenesis is primarily a cargo-driven process (FIG. 10). This is a critical advance for the field of exosome engineering, as success in engineering is always dependent upon a robust mechanistic understanding of the process to be engineered.

Highly enriched exosome cargo proteins appear to bud best when localized to the plasma membrane, and far worse when they are localized to the endosome membrane. This was established through observations that: (i) proteins that bud from cells efficiently can be found at discrete domains of the plasma membrane; (ii) plasma membrane targeting is in many cases sufficient to target oligomerized proteins into exosomes; (iii) plasma membrane anchors support the budding of exosome cargo proteins whereas endosome membrane anchors do not; (iv) CD81 and CD9 are the most highly enriched proteins in human exosomes and are localized at the plasma membrane, whereas CD63 is far less enriched and localized to endosomes; (v) redirecting CD9 from the plasma membrane to endosomes greatly reduced its exosomal secretion from the cells, (vi) redirecting CD63 from endosomes to the plasma membrane greatly increased its exosomal secretion from the cell; that; (vii) syntenin drives the biogenesis of CD63 exosomes by blocking CD63 endocytosis, (viii) clathrin-mediated and actin-dependent endocytosis inhibit exosome biogenesis, and (ix) high level expression of otherwise endocytosed cargoes like CD63 block all AP-2-mediated endocytosis and lead to their high level accumulation at the plasma membrane and drive their loading into exosomes. The present application shows that high-level expression of highly-enriched exosome carrier proteins results in 20-30-fold increases in exosome yield, indicating that exosome biogenesis is, at its most fundamental level, a cargo-driven process. In other words, while the working model of exosome biogenesis presented herein asserts that some exosomes are generated by a delayed pathway in which endocytosed cargoes drive the formation of intralumenal vesicles (ILVs), with some ILVs released as exosomes via endolysosomal exocytsosis rather than being destroyed in lysosomes, this is a secondary pathway that contributes relatively little to exosome biogenesis under normal conditions and is almost completely bypassed by any exosome engineering project that involves high-level expression of exosome cargo proteins.

The model presented herein draws a clear roadmap for the recombinant engineering of exosome protein content and exosome yield. In brief, there may be as few as three key steps to the process: (i) delivery of the protein to the plasma membrane, (ii) loading of the protein into exosomes, and (iii) maximizing its expression by the cell. If this roadmap is accurate, as well as the mechanistic model of exosome biogenesis, it should guide the production of exosomes that are loaded with a type-1 membrane protein that is not normally secreted from the cell in exosomes. Using SARS-CoV-2 as the test protein, this disclosure shows that the roadmap led to the production of Spike-display exosomes (S-DEX) and other display exosomes; and moreover, that S^{delta}-DEX exosomes elicited robust, protective immune responses at low dose, low cost, and without adjuvant, highlighting the potential of exosomes as a vaccine delivery platform.

### Example 11

### Methods

### Cell Lines, Culture, and Transfection

HEK293 cells were obtained from ATCC. 293F cells were obtained from Thermo. Cells were grown in either complete medium (Dulbecco's modified Eagle's medium high glucose with glutamine (Gibco Cat #11965118), containing 10% fetal bovine serum (Gibco Cat #26140079) and 1% penicillin/streptomycin solution (Gibco Cat #15140122)) at 37° C., 90% H₂O, and 5% CO₂, or in Freestyle medium (Gibco Cat #12338018) containing 1% penicillin/streptomycin solution at 110 rpm, 37° C., 90% H₂O, and 8% CO₂. Doxycycline-inducible, Tet-on derivatives of these cell lines (HtetZ, FtetZ, and FtetP) were generated by transfection with rtTAv16 expression vectors pJM1463 (expresses rtTAv16-2a-BleoR) or pJM1464 (expresses rtTAv16-2a-PuroR2), followed by selection with either zeocin or puromycin, respectively. Derivatives of Htet1, FtetZ, and FtetP cells were created by transfecting the cells lines with pITRSB-based Sleeping Beauty vectors carrying (i) an EFS-driven selectable marker gene and (ii) a TRE3G-driven gene encoding a form of SARS-CoV-2 Spike, as previously described (Guo et al., 2022). Transfections were performed using Lipofectamine 3000 (Invitrogen Cat #L3000001) according to the manufacturer's instructions, and the transgenic cell lines were selected as previously described (Guo et al., 2022). Zeocin was used at a concentration of 200 μg/mL and puromycin was used at a concentration of 3 μg/mL. For cell lines carrying GS-2a-BleoR constructs, complete medium was supplemented with a daily addition of 2 uM glutamate. Tet-on cell lines were induced in the presence of 1 μg/ml doxycycline.

### Plasmids

pJM1463 and pJM1464 were based on the pS vector described in Guo et al. (2021) and contain bicistronic ORFs encoding rtTAv16-2a-BleoR and rtTAv16-2a-PuroR2, respectively, downstream of the SFFV transcriptional control region. Plasmids designed to express S-D614G, S-D614G-2P, and S-D614G-2P-ΔC-ERES were based on the pC vector described in Guo et al. (2021) and contain spike genes downstream of the CMV transcriptional control region. Sleeping beauty transposons carrying an EFS-PuroR puromycin resistance gene and a TRE3G-spike gene were based on S149 (Quek et al., 2021) and generated by mutation of the spike gene in this vector. Sleeping beauty transposons carrying an EFS-GS-BleoR zeocin resistance gene and a TRE3G-spike gene were based on S149 and generated by swapping the PuroR ORF with the GS-2a-BleoR ORF, as well as mutation of the spike gene in this vector.

### Exosome Purification

For adherent cell cultures, 6 million cells were seeded onto 150 mm dishes in 30 ml of complete medium, allowed to adhere to the plates overnight, then incubated for three days in complete medium containing 1 μg/mL doxycycline. For suspension cell cultures, FtetZ, FtetP, and their spike-expressing derivatives, cells were seeded into 30 ml of Freestyle medium at a density of 1×10\hat{ }6 cells per ml and grown for 72 hours, with shaking. Culture media was collected and cells and cell debris were removed by centrifugation at 5,000 g at 4° C. for 15 minutes and passage through a 200 nm pore size diameter filtration unit. To collect exosomes by centrifugation, supernatants were spun for 30 minutes at 10,000×g, spun a second time for 30 minutes at 10,000×g, then spun at 100,000 g for 2 hours, all at 4° C. To collect exosomes by size exclusion chromatography and filtration, the 200 nm filtrate was concentrated ˜100-fold by centrifugal flow filtration across a 100 kDa pore size diameter filter (Centricon-70, MilliporeSigma), followed by size exclusion chromatography using qEV nano columns (Izon Sciences).

### Immunoblot

Cells were lysed in Laemmli/SDS-PAGE sample buffer lacking reducing agent. Samples were either maintained in reducing agent-free sample buffer or adjusted to 5% 8-mercaptoethanol, then heated to 100° C. for 10 minutes, and spun at 13,000×g for 2 minutes. Supernatants of these samples were then separated by SDS-PAGE and processed for immunoblots as previously described (Fordjour et al., 2022 and Fordjour et al., 2019).

### Antibodies and Drugs

For primary antibodies, anti-spike S1 (MM42) antibody was obtained from Sino Biological (Cat #40591-MM42), anti-spike S2 (1A9) antibody was obtained from Abcam (Cat #ab273433), anti-CD9 (HI9a) antibody was obtained from BioLegend (Cat #312102), anti-Hsp90 (F-8) antibody was obtained from Santa Cruz Biotechnology (Cat #sc-13119), and anti-alpha galactosidase A antibody was obtained from Proteintech (Cat #66121-1-Ig). For secondary antibodies, anti-human IgG (H+L) HRP conjugated antibody was obtained from Invitrogen (Cat #31410), and anti-mouse IgG HRP conjugated antibody was obtained from Cell Signaling (Cat #7076S). Doxycycline hyclate was obtained from PeproTech (Cat #2431450), zeocin was obtained from ThermoFisher (Cat #R25001), and puromycin was obtained from MilliporeSigma (Cat #P8833-25MG). To make A647-labeled anti-S2 antibody, 100 μg of anti-SARS-CoV-2/S2 (1A9) antibody was conjugated to A647 using the Lightning-Link Conjugation Kit (Abcam, Cat #ab269823) according to the manufacturer's protocol.

### Flow Cytometry

Cells were released by trypsinization and cell clumps were removed using a cell-strainer (Falcon Cat #352235). Approximately 500,000 cells were then concentrated by a brief spin at 13,000×g and resuspended in 100 μL of 4° C. FACS buffer (1% FBS in PBS) containing 2 mL of the A647 conjugated 1A9 antibody and incubated on ice in dark for 30 min with gentle mixing every 10 min. Cells were washed 3 times with 1 mL of 4° C. FACS buffer, with cells recovered by 500×g spin for 4 min at 4° C. After the final wash, cells were resuspended in 250 mL of chilled FACS buffer containing 0.5 mg/mL of DAPI, incubated on ice in the dark for 5 min, and analyzed using CytoFLEX S flow cytometer (Beckman Coulter). Cells were gated based on (1) FSC-A vs SSC-A (P1), (2) FSC-A vs FCS-H (P2), (3) FSC-A vs PB450-A (P3), and (4) FSC-A vs APC-A (P4). Approximately 20,000 singlet, live cells (after gate P3) were recorded on the APC channel (A647 fluorescence), and the positive signals (after gate P4) were analyzed by % parent (P4/P3) and mean APC-A at P4.

### Electron Microscopy

Electron microscopy grids were obtained from (Electron Microscopy Sciences) and pre-charged at negative glo for 30 seconds using a GloQube Plus Glow Discharge system (Electron Microscopy Sciences). These charged grids were incubated with exosome samples for 2 minutes followed by three washes, then stained twice in 1% uranyl acetate for 2 minutes each. Grids were then dried by brief vacuum aspiration and subsequently imaged on a Hitachi 7600 transmission electron microscope.

### Formal Analysis

Quantitative results were evaluated using Student's unpaired t-test or ANOVA, assuming Gaussian distribution and equal variances in GraphPad Prism.

### Example 12

### Human Cells do not Secrete Spike on Exosomes

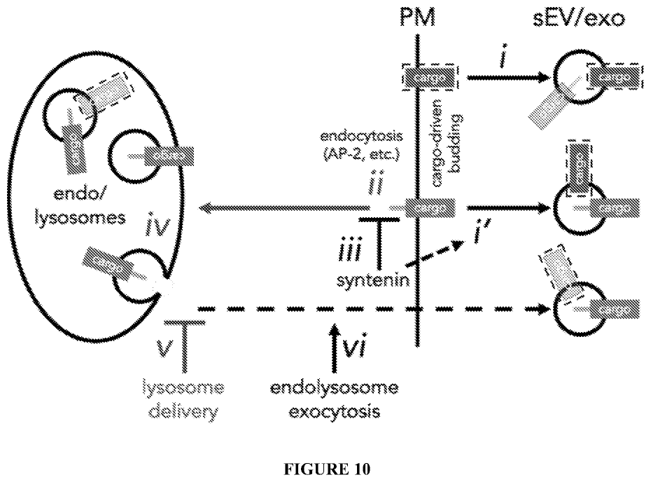

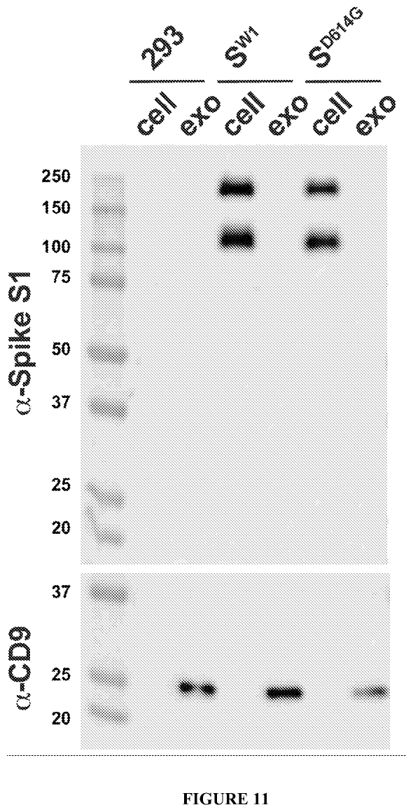

The first essential step in the interrogation of exosome engineering roadmap was to identify a type-1 membrane protein that is not released from the cell in exosomes. The SARS-CoV-2 Spike protein was used, in part because it is of high biomedical significance, in part because an exosome-based Spike vaccine may be of clinical utility, and in part because there is a parallel study on the cell biology of Spike protein biogenesis and trafficking within the cell (Guo et al., 2022). In brief, that parallel study established that Spike is a lysosome membrane protein, and that D614G and other key mutations enhance the lysosomal trafficking of Spike. Using stable Tet-On, HEK293-derived cell lines that carry doxycycline-inducible, Spike-expressing transgenes, this disclosure shows that the Spike protein encoded by the original Wuhan-1 strain of SARS-CoV-2 is not released from the cell in exosomes, and that neither is the D614G form of Spike that arose shortly after the start of the pandemic (FIG. 11) (all current and former strains of concern carry the D614G mutation). The presence of Spike at two sizes reflects its processing by furin at the S1/S2 boundary due to the furin cleavage site insertion mutation (FCSI) that at or just prior the virus' zoonotic leap into the human population.

### Example 13

Redirecting Spike from Lysosomes to the Plasma Membrane

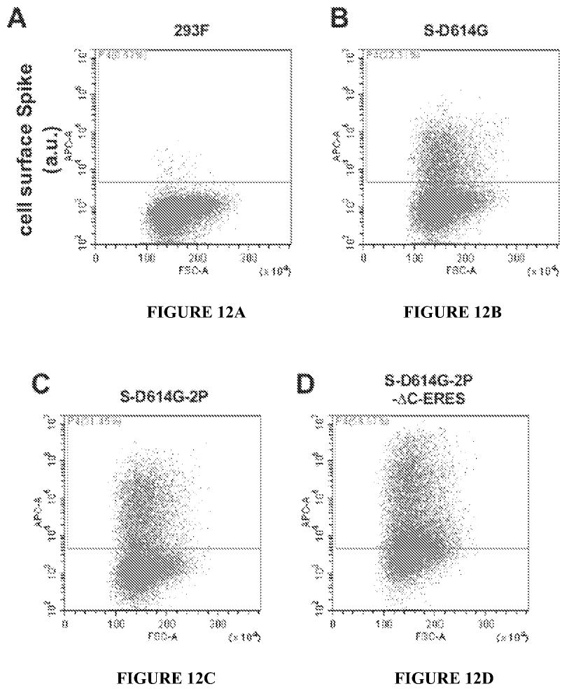

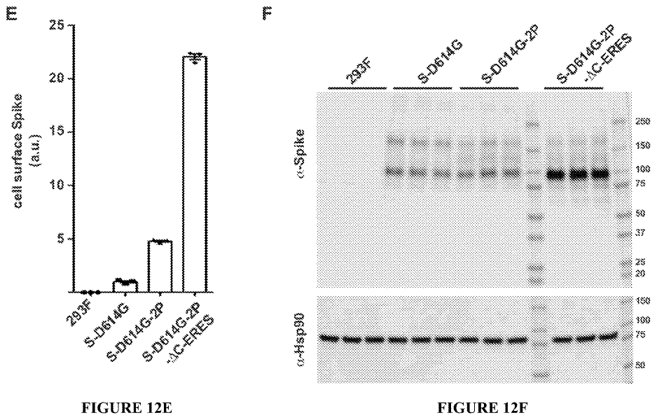

The analysis of Spike protein trafficking established that the lysosomal trafficking information in Spike is localized to its extracellular domain (ECD), that mutations in the Spike ECD can disrupt Spike protein trafficking to lysosomes, and that mutations that disrupt Spike trafficking to lysosomes lead to an accumulation of Spike expression at the cell surface. Given that the roadmap of exosome engineering highlights the need to direct all protein to the plasma membrane while the more specific goal is to make Spike display exosomes as a SARS-CoV-2 vaccine, the Spike protein was mutated in a way that might (i) disrupt the lysosome sorting information in Spike and thereby increase Spike's delivery to the plasma membrane, while also (ii) increasing Spike's immunogenicity. The Spike mutations most likely to have these dual effects are the 986KV987-to-986PP987 diproline (2P) substitutions. The 2P substitutions enhance Spike immunogenicity and the production of virus-neutralizing antibodies, dramatically alter ECD structure by locking Spike proteins in their trimeric, prefusion conformation, and are present in all FDA-approved Spike vaccines. Surprisingly, the present disclosure shows that 2P substitutions increased cell surface Spike expression by ˜500% (FIGS. 12A, 12B, 12C, 12E).

While the 2P substitutions alone had a dramatic effect on Spike protein trafficking, previous studies have established that Spike also carries an ER retrieval signal in its cytoplasmic C-terminal tail (CTT) peptide, a motif that binds to COP-I, the machinery that mediates the return of escaped proteins back to the ER. Given that the delivery of a type-1 membrane protein to the plasma membrane requires its efficient export from the ER, the robust cell surface expression of S^{D614G}-2P should be further increased by replacing the COPI-binding CTT of Spike with a different CTT peptide carrying a diacidic ER export signal (ERES), which binds the COPII machinery involved in ER-to-Golgi protein transport. The protein S^{D614G}-2P-ΔC-ERES has precisely this structure and displayed 22-fold higher cell surface expression than S^{D614G} and 4-fold higher cell surface expression than S^{D614G}-2P (FIGS. 12A-12E). Immunoblot analysis revealed that the 2P substitution had no effect on Spike expression, that the ΔC-ERES substitutions increased Spike abundance by only 2-fold, and that the primary reason for their differences in cell surface expression reflected a redirection of Spike from internal compartments of the cell to plasma membrane (FIG. 12F).

### Example 14

### FtetZ/S^{delta}-CSM-2P-ΔC-ERES Cells Produce Spike-Display Exosomes

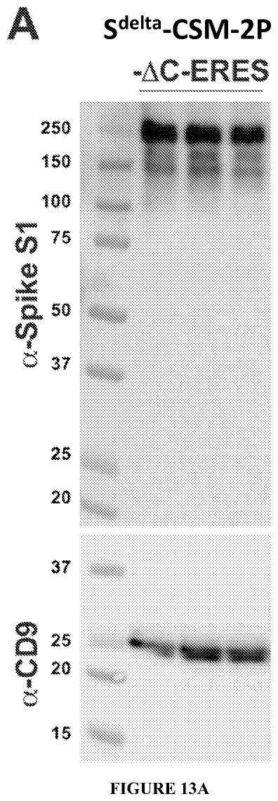

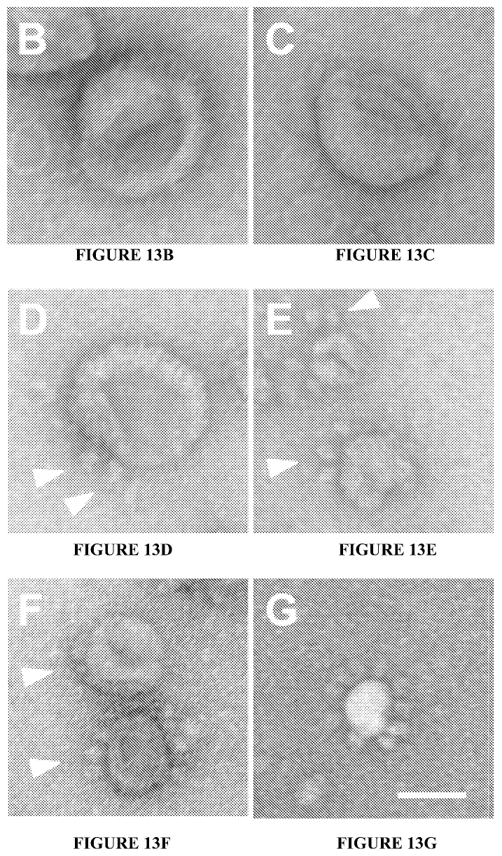

This disclosure sets forth a roadmap for exosome engineering, a mechanism of exosome biogenesis, and compositions and methods to produce Spike display exosomes that can be used as a safe, effective, and inexpensive vaccine for SARS-CoV-2. The 2P and ΔC-ERES substitutions were combined with a furin cleavage site mutation (CSM; 682RRAR685-to-682GSAG685) that eliminates the biogenic processing of full-length Spike into its S1 and S2 components, thereby eliminating the potential loss of the S1 fragment, which carries the N-terminal domain and receptor binding domain to which most neutralizing antibodies bind. Into this increasingly mutated Spike transgene, all of the selected Spike mutations present in the delta strain of the SARS-CoV-2 (T19R, G142D, D157-158, L452R, T478K, D614G, P681R, and D950N) were also inserted. This protein, S^{delta}-CSM-2P-ΔC-ERES, was then expressed from a stably-inserted, doxycycline-inducible transgene delivered on a Sleeping Beauty transposon into a Tet-on derivative of 293F cells (FtetZ). These cells were grown for 3 days in chemically-defined media, in triplicate culture, supplemented with doxycycline to induce Spike protein expression. Three days later, exosomes were purified from these cultures and interrogated by immunoblot and by electron microscopy. These three independent exosome preparations each contained full-length S^{delta}-CSM-2P-ΔC-ERES, un-cleaved due to the CSM mutation, as well as the exosome marker protein CD9 (FIG. 13A). Negative stain electron microscopy revealed that exosomes from the control 293F cell culture had the typical appearance of human exosomes (FIGS. 13B, 13C), whereas some of the exosomes secreted by the S^{delta}-CSM-2P-ΔC-ERES-expressing cell line displayed Spike trimer structures emanating from the exosome surface (FIGS. 13D-13G). These results validated the basic outline of the roadmap presented herein. Because only ˜10% of the exosomes released by this cell line (FtetZ/CG473) carried a Spike trimer on their surface, the design of the protein, transgene, vector, and cell line had room for improvement.

### Example 15

### Validation of Enhanced Type-1 Exosome Membrane Anchor (T1EMA) Peptides

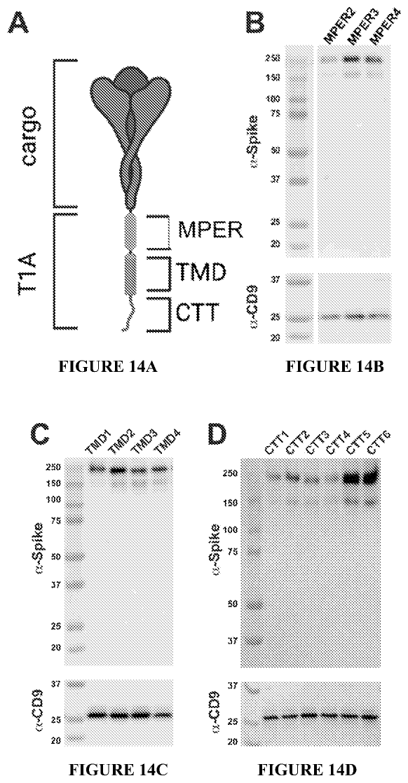

The loading of Spike proteins into exosomes was improved upon, while type-1 exosome membrane anchors (T1EA) comprised of a non-Spike membrane-proximal external region (MPER), transmembrane domain (TMD), and carboxy-terminal tail (CTT) were also developed (FIG. 14A). When the 13 amino acid-long Spike MPER peptide (-DLQELGKYEQYIK-) was replaced with paralogous segments from the proteins HIV ENV (membrane-proximal external region of the human immunodeficiency virus type 1, Zwick et al., 2005), MLV ENV (murine leukemia virus envelope glycoprotein, Salamango et al., 2016), and VSVG (vesicular stomatitis virus glycoprotein, Rose et al., 1980), all three forms of Spike were still secreted from the cell in exosomes (FIG. 14B), though the MLV ENV-derived peptide appeared to support the highest level of Spike loading into exosomes. Replacement of the Spike TMD revealed that those from the type-1 exosomal membrane proteins from the immunoglobulin superfamily IgSF2, IgSF3, and IgSF8 also supported the exosomal loading of Spike (FIG. 14C). As for the CTT, each of the ERES-containing replacements (CTT2-CTT6) supported the vesicular secretion of Spike, but those from the Carajas virus G protein (CTT5) or its fusion with the Golgi export signal of reovirus p14 (CTT6) appeared to increase the amount of Spike loaded into exosomes (FIG. 13D). These exosomal forms of Spike migrated at the expected size of a Spike trimer even though the samples had been boiled in SDS-PAGE sample buffer prior to loading on the gels.

### Example 16

Improved Spike Expression Leads to Improved Loading of Spike into Exosomes

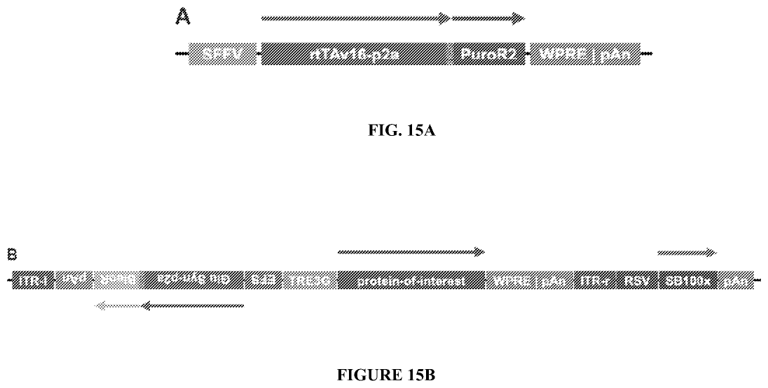

As presented above, novel antibiotic resistance genes can augment the amount of protein expressed from integrated transgenes. These advances were used to create a new vector (FIG. 15A) designed to express the doxycycline-regulated rtTAv16 transcription factor from a polycistronic ORF linked to the viral p2a peptide and the PuroR2 gene encoding a novel puromycin acetyltransferase gene. This allowed modification of the pITRSB Sleeping Beauty vector previously used for driving the doxycycline-induced expression of Spike proteins. The PuroR gene was removed and replaced with a new, bicistronic selectable marker gene not previously described, GS-2a-BleoR (FIG. 15B), which encodes (i) human glutamine synthetase, (ii) the porcine teschovirus 2a peptide, and (iii) BleoR. This new selectable marker combines the most restrictive antibiotic resistance gene, BleoR, with a metabolic selectable marker gene, glutamine synthetase (GS), which can complement the auxotrophy of glutamine synthetase-deficient (i.e. GLUL^{-/-}) cell lines, confer resistance to the GS inhibitor methionine sulfoxamine, while at the same time providing cells with high levels of glutamine. The increased bioavailability of glutamine is important, as it feeds into the most common metabolic demands of all cell culture systems regardless of whether GS is being used as a selectable marker.

As a preliminary test of this new system, 293F cells were transfected with the plasmid pJM1464 (FIG. 15A), selected for puromycin-resistant cells, and the resulting clones were pooled to create the cell line FtetP. Next, these cells were transfected with pITRSB-based vectors (FIG. 15B) in which the selectable marker gene encoded either BleoR or GS-2a-BleoR, and the ‘protein-of-interest’ was mCherry. Two days later, cells were switched to culture medium containing zeocin and supplemented daily with 3 mM glutamate, followed by pooling of all antibiotic-resistant clones from each transfection to make two cell lines that differed only in the selection by the BleoR or GS-2a-BleoR selectable marker genes. Measurement of mCherry expression levels in these cell lines by flow cytometry revealed that GS-2a-BleoR-selected cell line expressed a ˜2.7-fold higher level of mCherry (mCherry fluorescence brightness 131,216 for BleoR vs 357,672 for GS-2a-BleoR selectable marker). Furthermore, when these two cell lines were cultured in complete medium lacking zeocin but containing 100 uM methionine sulfoxamine, the BleoR-selected cells died whereas the GS-2a-BleoR-selected cell lines survived; and moreover, that passaging the cells in increasing concentrations of methionine sulfoxamine led to progressively higher levels of mCherry expression.

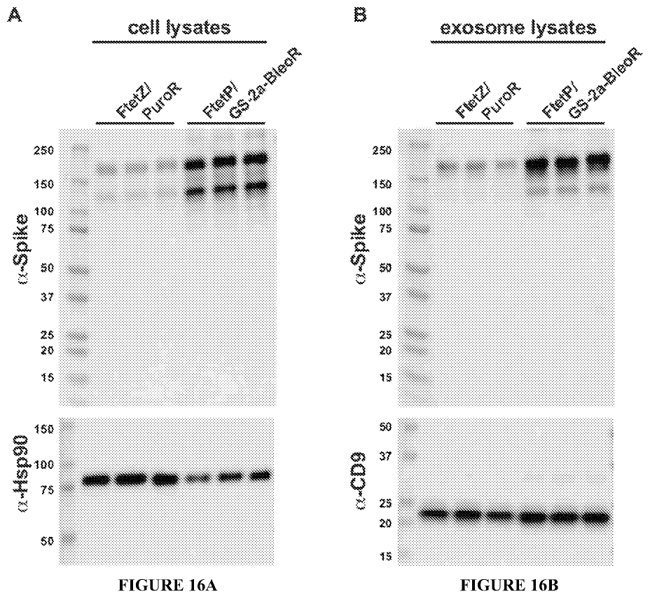

To determine whether the FtetP/GS-2a-BleoR cell transgenesis system led to increased expression of Spike and increased amounts of Spike in exosomes relative to the FtetZ/PuroR transgenesis system, a Sleeping Beauty transposon was designed to express an exosomal form of S^{delta}-CSM-2P in both transposons, the PuroR-based transposon was transfected into FtetZ cells, and the GS-2a-BleoR-based transposon was transfected into FtetP cells. Single cell clones were selected in media containing the appropriate antibiotic, followed by pooling the cells from each transfection to make two distinct cell lines. Triplicate cultures of these two cell lines were grown in parallel in chemically defined media containing 1 μg/mL doxycycline for three days, with shaking, after which cells and exosomes were collected and interrogated by immunoblot. The FtetP/GS-2a-BleoR system selected for a 3-fold increase in Spike expression within the cell and a 5-fold increase in the amount of Spike present in exosomes (FIG. 16). Once again, it is interesting to note that the exosome-associated forms of Spike migrated at the expected size of a Spike trimer whereas the cell-associated Spike had a higher proportion of monomeric Spike.

### Example 17

### Enhanced Production of Spike Display Exosomes

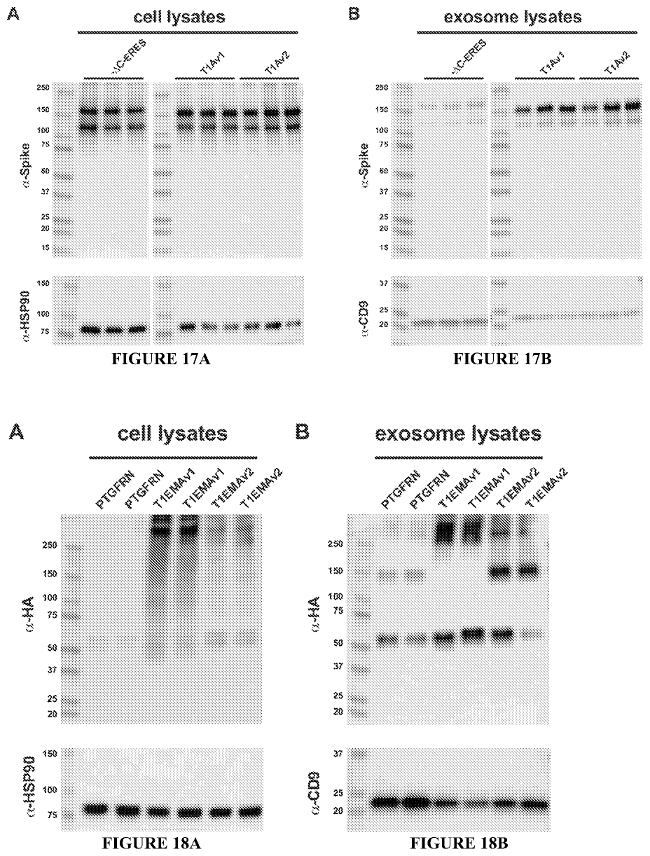

Our next experiment was to measure the relative expression and exosomal secretion of different anchored forms of S^{delta}-CSM-2P at the elevated levels afforded by the FtetP/GS-2a-BleoR transgene expression system. Specifically, FtetP cells carrying GS-2a-BleoR-selected transposons encoding S^{delta}-CSM-2P-ΔC-ERES, S^{delta}-CSM-2P-T1EMAv1, or S^{delta}CSM-2P-T1EMAv2 were created. T1EMAv1 combines the MLV MPER, IgSF3 TMD, and CTT6, while T1EMAv2 combines the MLV MPER, IgFS8 TMD, and CTT6. These three cell lines were grown in triplicate in CDM containing doxycycline and glutamate, with shaking, for 3 days, followed by collection of cells and exosomes, and interrogation of these samples by IB. All three proteins were expressed to similar levels (FIG. 17A), demonstrating that the different MPER-TMD-CTT sequences had little to no effect on the overall expression of these type-1 anchored S^{delta}-CSM-2P proteins. However, the S^{delta}-CSM-2P-T1EMAv1 and S^{delta}-CSM-2P-T1EMAv2 were both loaded into exosomes ˜5-fold more efficiently than S^{delta}-CSM-2P-ΔC-ERES (FIG. 17B). In addition, when these exosomes were interrogated by negative stain electron microscopy, morphologically recognizable Spike structures were observed on 50% of exosomes released by cells expressing the S^{delta}-CSM-2P-T1EMAv2, a significant increase over the percentage of exosomes carrying Spike in the initial Spike-display exosomes (see FIG. 13).

### Example 18

### Exosome Display of Influenza Hemagglutinin (HA)

To determine whether the combination of the T1EMAv2 anchor and FtetP/GS-2a-BleoR expression system can be used to display other viral antigens on the exosome surface, and to compare its utility to that the of PTGFRN-derived exosome membrane anchor described by Dooley et al. (2021), pITRSB/GS-2a-BleoR transposons were designed to express the extracellular domain of influenza hemagglutinin (HA) fused to the PTGFRN C-terminal domain, T1EMAv1, and T1EMAv2. These transposons were transfected into FtetP cells followed by selection of zeocin-resistant cell clones, and pooling of clones from each transfection to generate three polyclonal cell lines. These cells were grown side-by-side in duplicate cultures in CDM supplemented with doxycycline and glutamate, with shaking, for three days. Cells and exosomes were then collected and processed by immunoblot (FIG. 18). These experiments revealed that the highest levels of exosomal HA were achieved using the T 1EMAv2 anchor, and that the exosomal forms of HA appeared to include monomers, a particularly high level of trimers, and also some higher molecular mass forms of HA. Although the T1EMAv1 anchor appears to have loaded more HA material in total, none of this migrated at the expected mass of HA trimers. The PTGFRN-anchored form of HA was expressed poorly, and while its relative loading may have been high (i.e. E/C ratio), the exosomes produced by this cell line had the lowest overall levels of HA protein.

### Example 19

### Exosome Display of VEGFR, GLA, and Trastuzumab.

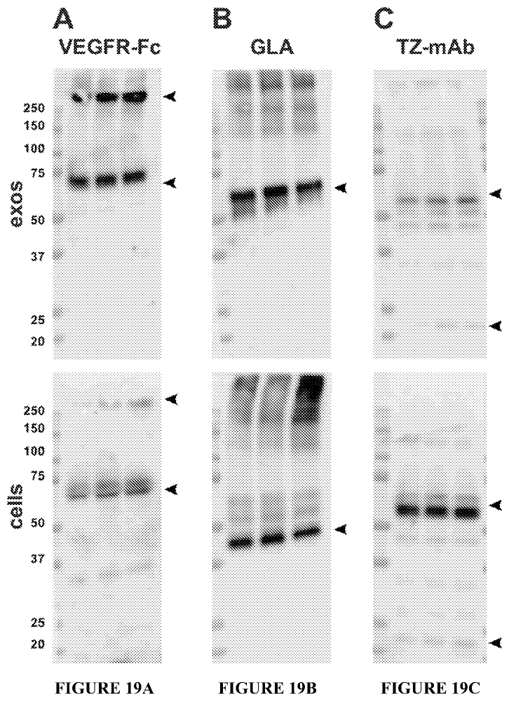

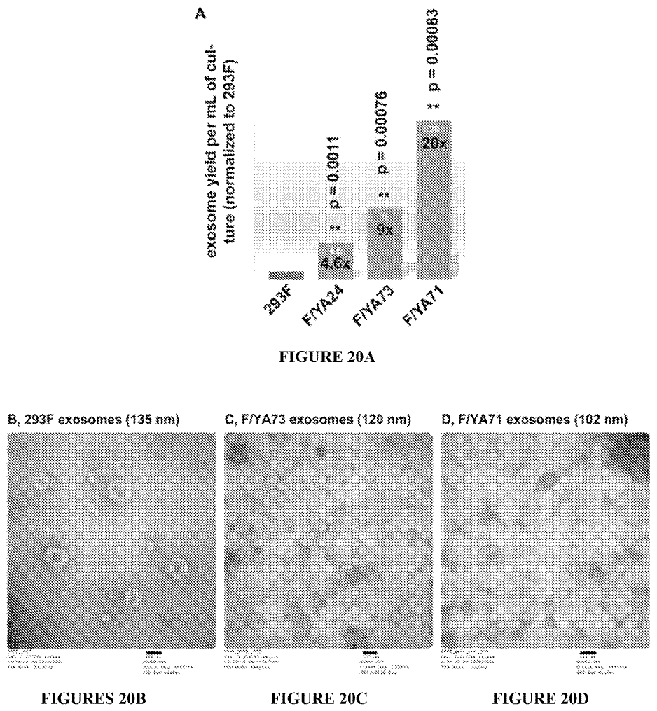

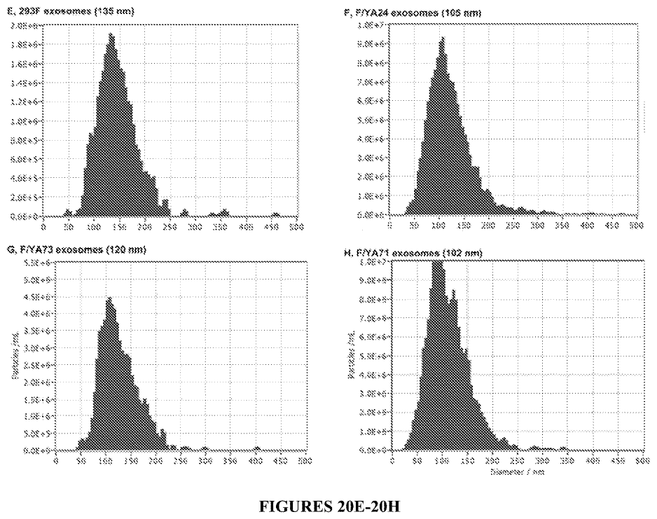

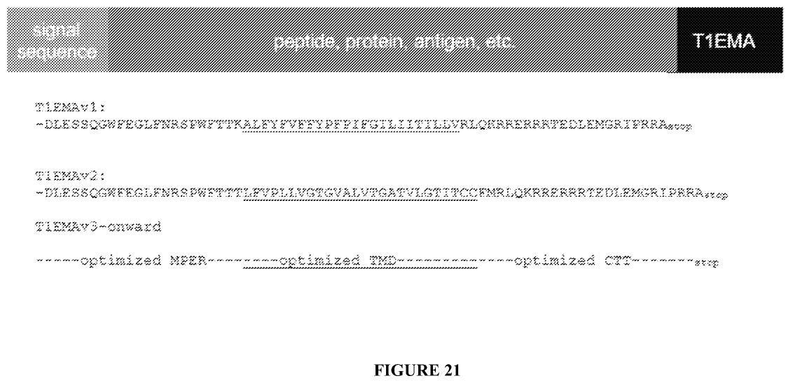

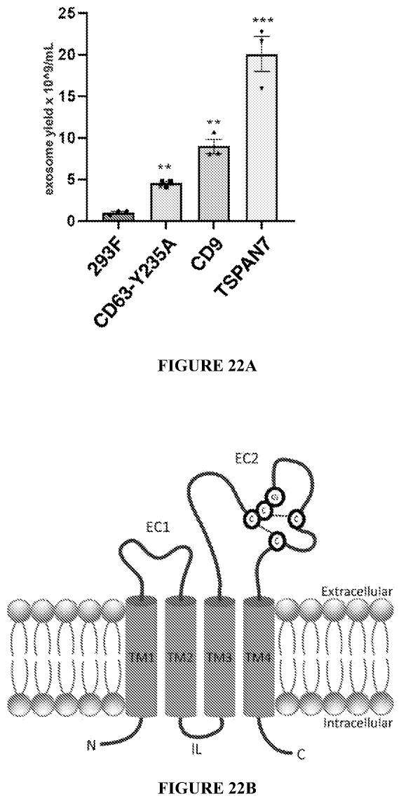

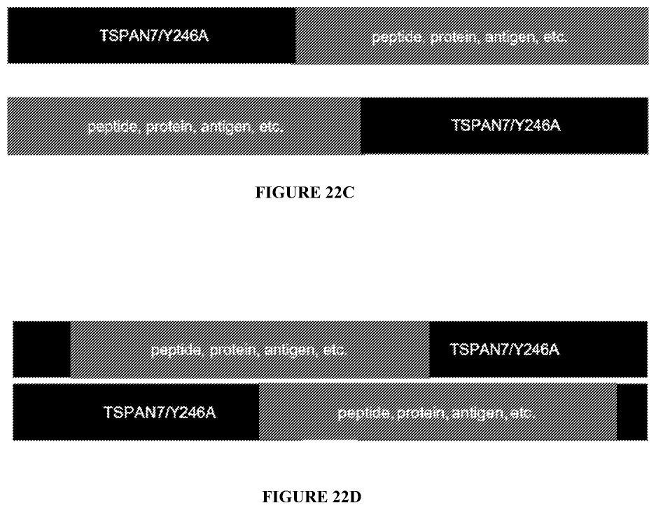

Next, this system was tested to verify whether it would support the exosome display of non-viral cargoes, and more specifically, non-viral proteins of potential clinical utility. Toward this end, FtetP/GS-2a-BleoR expression vectors were designed to express T1EMAv2 fusion proteins to (i) the extracellular domain of vascular endothelial growth factor receptor (VEGFR) fused to the constant region of the human immunoglobulin heavy chain (Fc domain of human IgG), (ii) the lysosomal enzyme alpha galactosidase A (GLA), and (iii) the heavy chain of the HER2-inhibiting monoclonal antibody trastuzumab, co-expressed with a third gene encoding an unaltered form of the trastuzumab light chain. These transposons were transfected into FtetP cells followed by selection of zeocin-resistant cells, and pooling of all zeocin-resistant clones from each transfection to create four cell lines designed for the doxycycline-induced expression of their encoded proteins. These cell lines were grown side-by-side in 3 independent cultures in CDM supplemented with doxycycline and glutamate, with shaking for three days. Cells and exosomes were then collected and processed by immunoblot (FIG. 19). All three of these proteins were detected in the exosome fraction, confirming that the expression system and synthetic exosome membrane anchors described in this study can also be used to generate VEGFR-display exosomes, GLA-display exosomes, and trastuzumab-display exosomes.

### Example 20

### Discussion

The production of highly engineered exosomes of defined content and function requires a robust mechanistic understanding of exosome biogenesis, and especially how cargo proteins are loaded into exosomes. The instant application shows that exosome biogenesis occurs primarily at the plasma membrane, that loading of cargo proteins into exosomes is inhibited strongly by endocytosis, and that the delayed, endosome-dependent pathway of ILV secretion is at best a secondary pathway that is refractory to high level engineering. Furthermore, the model of exosome biogenesis presented here (FIG. 10) draws a roadmap of exosome engineering in which the three key considerations are (i) delivery of the protein to the plasma membrane, (ii) loading of the protein into exosomes, and (iii) maximizing its expression by the cell.

### Improved Type-1 Exosome Membrane Anchors

Recombinant exosome engineering takes many forms. For example, nearly any protein can be loaded into exosomes by a combination of high-order oligomerization and plasma membrane associated peptides, and synthetic cargoes combining these features can be efficiently loaded into the lumen of exosomes. However, genetically encoded oligomerization of type-1 membrane proteins often results in their retention in the ER and/or degradation by the ER-associated protein degradation pathway (ERAD), limiting the utility of this approach for modifying the outer surface of the exosome membrane. Furthermore, while highly enriched tetraspanins can be used to deliver peptides and certain protein domains to the cell surface, these polytopic fusion proteins are not useful for displaying proteins in a type-1 topology.

These limitations can be overcome by inventing two type-1 exosome membrane anchors, T1EMAv1 and T1EMAv2, that can be used to load proteins into the membrane of nascent budding exosomes in a type-1 topology. Specifically, T1EMA peptides were built by combining validated ER export signals, adding Golgi export signals into their carboxy-terminal tails, combining these with transmembrane domains from known exosomal membrane proteins, and a membrane-proximal region from the MLV ENV proteins that enhances ENV loading into the Trojan exosomes that are MLV virus particles. These advances, together with the empirical utility of T1EMAv1 and v2 peptides, set the stage for further improvement in T 1EMA activity through screening for sequence variants that enhance cargo trafficking from the ER to the plasma membrane and/or increase the efficiency of cargo-T1EMA loading into secreted vesicles. In side-by side studies, the T1EMAv2 peptide supported vastly superior loading of influenza HA onto the exosome surface. The difference was minimal, however, for loading Spike onto the exosome surface.

### Importance of Minimizing Competing Sorting Information

The production of Spike display exosomes and the S-DEX vaccine demonstrated a second key variable in the production of recombinantly engineered exosomes; namely, the elimination of competing protein trafficking information from the cargo protein of interest. In the case of Spike, two distinct sorting pathways dominate its biogenesis within human cells and its trafficking to lysosomes, which is mediated by its extracellular domain and its retention in the ER via interaction of its C-terminal peptide with the COPI machinery. A diproline substitution incorporated into the extracellular domain of all Spike vaccines disrupts Spike trafficking to lysosomes; this mutation alone increased the cell surface expression of Spike by ˜5-fold. Moreover, when Spike's ER retrieval signal was deleted and replaced with an ER export signal, a further 4-fold increase was induced in the cell surface expression of Spike, with the combined effect of both mutations increasing the cell surface expression of Spike by >20-fold.

Enhanced Cargo Expression Further Increases Cargo Loading into Exosomes

The operating hypothesis (FIG. 10) is that exosome biogenesis is a cargo driven process in which vesicle biogenesis responds to the level of cargo protein expression. This led to development of optimized selectable marker genes and transgene delivery systems for high-level transgene expression. Moreover, to develop systems for clinical exosome production, the many advantages discovered regarding choice of antibiotic resistance gene on high-level protein expression were combined with the many advantages of glutamine synthetase as a selectable marker and methionine sulfoxamine as a selective agent.

This combination was brought to fruition in the GS-2a-BleoR selectable marker gene. Moreover, FtetP cells, generated by linking expression of the rtTAv16 transcription factor to the PuroR2 gene, then transduced with GS-2a-BleoR-linked, Spike-expressing transgenes and selected in zeocin-containing media, did indeed express significantly higher levels of Spike, and more importantly, loaded a disproportionately higher level of Spike in their exosomes. Because the GS-2a-BleoR gene also conferred resistance to methionine sulfoxamine, even higher levels of linked transgene expression might be obtained by continued culture in increasing concentrations of this glutamine synthetase inhibitor. This approach might also allow for the unanticipated benefits of methionine sulfoxamine selection that accrue due to secondary metabolic changes that amplify the production of proteins that depend on the host secretory pathway.

### Extension to Other Cargo Proteins

The T1EMAv2 peptide loaded influenza HA into the exosome membrane, and surprisingly, this peptide anchor sequence loaded significantly more HA antigen into the exosomes than the C-terminal fragment of PTGFRN, a member of the IgSF-EW1 immunoglobulin superfamily described by Dooley et al. (2021) for enrichment of extracellular vesicles. (The single transmembrane domain of PTGFRN contains an EW1 (glutamic acid-tryptophan-isoleucine) motif.) Additionally, the T1EMAv2 anchor peptide can decorate the exosome surface with proteins as diverse as a receptor trap protein based on the FDA-approved angiogenesis inhibitor aflibercept (a VEGFR-Fc fusion protein), the FDA approved lysosomal enzyme GLA (for treatment of Fabry disease), and the FDA approved breast cancer therapeutic trastuzumab (an inhibitory anti-HER2 monoclonal antibody). These results indicate that the T1EMA approach is broadly useful, and indicates that further improvements in T1EMA efficacy will lead to even more efficient production of exosomes of defined content and function.

### REFERENCES

Boni, M. F., Lemey, P., Jiang, X., Lam, T. T., Perry, B. W., Castoe, T. A., Rambaut, A., and Robertson, D. L. (2020). Evolutionary origins of the SARS-CoV-2 sarbecovirus lineage responsible for the COVID-19 pandemic. Nat Microbiol 5, 1408-1417. 10.1038/s41564-020-0771-4.
Boshart, M., Weber, F., Jahn, G., Dorsch-Hasler, K., Fleckenstein, B., and Schaffner, W. (1985). A very strong enhancer is located upstream of an immediate early gene of human cytomegalovirus. Cell 41, 521-530. 10.1016/s0092-8674(85)80025-8.
Chalberg, T. W., Portlock, J. L., Olivares, E. C., Thyagarajan, B., Kirby, P. J., Hillman, R. T., Hoelters, J., and Calos, M. P. (2006). Integration specificity of phage phiC31 integrase in the human genome. J Mol Biol 357, 28-48. 10.1016/j.jmb.2005.11.098.
Dhara, V. G., Naik, H. M., Majewska, N. I., and Betenbaugh, M. J. (2018). Recombinant Antibody Production in CHO and NSO Cells: Differences and Similarities. BioDrugs 32, 571-584. 10.1007/s40259-018-03 19-9.
Diamond, M., Chen, R., Xie, X., Case, J., Zhang, X., VanBlargan, L., Liu, Y., Liu, J., Errico, J., Winkler, E., et al. (2021). SARS-CoV-2 variants show resistance to neutralization by many monoclonal and serum-derived polyclonal antibodies. Res Sq. 10.21203/rs.3.rs-228079/v1.
Donello, J. E., Loeb, J. E., and Hope, T. J. (1998). Woodchuck hepatitis virus contains a tripartite posttranscriptional regulatory element. J Virol 72, 5085-5092. 10.1128/JVI.72.6.5085-5092.1998.
Dong, X., Lei, Y., Yu, Z., Wang, T., Liu, Y., Han, G., Zhang, X., Li, Y., Song, Y., Xu, H., et al. (2021). Exosome-mediated delivery of an anti-angiogenic peptide inhibits pathological retinal angiogenesis. Theranostics 11, 5107-5126. 10.7150/thno.54755.
Fonseca, J. P., Bonny, A. R., Kumar, G. R., Ng, A. H., Town, J., Wu, Q. C., Aslankoohi, E., Chen, S. Y., Dods, G., Harrigan, P., et al. (2019). A Toolkit for Rapid Modular Construction of Biological Circuits in Mammalian Cells. ACS Synth Biol 8, 2593-2606. 10.1021/acssynbio.9b00322.
Fordjour, F. K. D., G. G., and Gould, S. J. (2019). A shared pathway of exosome biogenesis operates at plasma and endosome membranes. BiorXiv, 1-42. https://doi.org/10.1101/545228.
Gao, X., Ran, N., Dong, X., Zuo, B., Yang, R., Zhou, Q., Moulton, H. M., Seow, Y., and Yin, H. (2018). Anchor peptide captures, targets, and loads exosomes of diverse origins for diagnostics and therapy. Sci Transl Med 10. 10.1126/scitranslmed.aat0195.
Gaspar, V., de Melo-Diogo, D., Costa, E., Moreira, A., Queiroz, J., Pichon, C., Correia, I., and Sousa, F. (2015). Minicircle DNA vectors for gene therapy: advances and applications. Expert Opin Biol Ther 15, 353-379. 10.1517/14712598.2015.996544.
Gatignol, A., Durand, H., and Tiraby, G. (1988). Bleomycin resistance conferred by a drug-binding protein. FEBS Lett 230, 171-175. 10.1016/0014-5793(88)80665-3.
Ghilardi, N., Pappu, R., Arron, J. R., and Chan, A. C. (2020). 30 Years of Biotherapeutics Development-What Have We Learned? Annu Rev Immunol 38, 249-287. 10.1146/annurev-immunol-101619-031510.
Gruss, P., Lai, C. J., Dhar, R., and Khoury, G. (1979). Splicing as a requirement for biogenesis of functional 16S mRNA of simian virus 40. Proc Natl Acad Sci USA 76, 4317-4321. 10.1073/pnas.76.9.4317.
Guo, C., Fordjour, F. K., Tsai, S. J., Morrell, J. C., and Gould, S. J. (2021). Choice of selectable marker affects recombinant protein expression in cells and exosomes. J Biol Chem, 100838. 10.1016/j.jbc.2021.100838.
Iwamoto, M., Bjorklund, T., Lundberg, C., Kirik, D., and Wandless, T. J. (2010). A general chemical method to regulate protein stability in the mammalian central nervous system. Chem Biol 17, 981-988. 10.1016/j.chembiol.2010.07.009.
Joyner, A., Yamamoto, Y., and Bernstein, A. (1982). Retrovirus long terminal repeats activate expression of coding sequences for the herpes simplex virus thymidine kinase gene. Proc Natl Acad Sci USA 79, 1573-1577. 10.1073/pnas.79.5.1573.
Kaster, K. R., Burgett, S. G., Rao, R. N., and Ingolia, T. D. (1983). Analysis of a bacterial hygromycin B resistance gene by transcriptional and translational fusions and by DNA sequencing. Nucleic Acids Res 11, 6895-6911. 10.1093/nar/11.19.6895.
Kimura, M., Kamakura, T., Tao, Q. Z., Kaneko, I., and Yamaguchi, I. (1994). Cloning of the blasticidin S deaminase gene (BSD) from Aspergillus terreus and its use as a selectable marker for Schizosaccharomyces pombe and Pyricularia oryzae. Mol Gen Genet 242, 121-129. 10.1007/BF00391004.
Kjaer, J., and Belsham, G. J. (2018). Modifications to the Foot-and-Mouth Disease Virus 2A Peptide: Influence on Polyprotein Processing and Virus Replication. J Virol 92. 10.1128/JVI.02218-17.
Luke, G., Escuin, H., De Felipe, P., and Ryan, M. (2010). 2A to the fore—research, technology and applications. Biotechnol Genet Eng Rev 26, 223-260. 10.5661/bger-26-223.
Miyazaki, Y., Imoto, H., Chen, L. C., and Wandless, T. J. (2012). Destabilizing domains derived from the human estrogen receptor. J Am Chem Soc 134, 3942-3945. 10.1021/ja209933r. Mulligan, R. C., and Berg, P. (1980). Expression of a bacterial gene in mammalian cells. Science 209, 1422-1427. 10.1126/science.6251549.
Mulsant, P., Gatignol, A., Dalens, M., and Tiraby, G. (1988). Phleomycin resistance as a dominant selectable marker in CHO cells. Somat Cell Mol Genet 14, 243-252. 10.1007/BF01534585.
Ochmann, M. T., and Ivics, Z. (2021). Jumping Ahead with Sleeping Beauty: Mechanistic Insights into Cut-and-Paste Transposition. Viruses 13. 10.3390/v13010076.
Ohno, H., Stewart, J., Fournier, M. C., Bosshart, H., Rhee, I., Miyatake, S., Saito, T., Gallusser, A., Kirchhausen, T., and Bonifacino, J. S. (1995). Interaction oftyrosine-based sorting signals with clathrin-associated proteins. Science 269, 1872-1875. 10.1126/science.7569928. Okayama, H., and Berg, P. (1992). A cDNA cloning vector that permits expression of cDNA inserts in mammalian cells. 1983. Biotechnology 24, 270-279.
Pegtel, D. M., and Gould, S. J. (2019). Exosomes. Annu Rev Biochem 88, 487-514. 10.1146/annurev-biochem-013118-111902.
Pols, M. S., and Klumperman, J. (2009). Trafficking and function of the tetraspanin CD63. Exp Cell Res 315, 1584-1592. 10.1016/j.yexcr.2008.09.020.
Rakhit, R., Navarro, R., and Wandless, T. J. (2014). Chemical biology strategies for posttranslational control of protein function. Chem Biol 21, 1238-1252. 10.1016/j.chembiol.2014.08.011.
Shaner, N. C., Campbell, R. E., Steinbach, P. A., Giepmans, B. N., Palmer, A. E., and Tsien, R. Y. (2004). Improved monomeric red, orange and yellow fluorescent proteins derived from Discosoma sp. red fluorescent protein. Nat Biotechnol 22, 1567-1572. 10.1038/nbt1037. Southern, P. J., and Berg, P. (1982). Transformation of mammalian cells to antibiotic resistance with a bacterial gene under control of the SV40 early region promoter. J Mol Appl Genet 1, 327-341.
Tipanee, J., VandenDriessche, T., and Chuah, M. K. (2017). Transposons: Moving Forward from Preclinical Studies to Clinical Trials. Hum Gene Ther 28, 1087-1104. 10.1089/hum.2017.128.
Turan, S., Zehe, C., Kuehle, J., Qiao, J., and Bode, J. (2013). Recombinase-mediated cassette exchange (RMCE)—a rapidly-expanding toolbox for targeted genomic modifications. Gene 515, 1-27. 10.1016/j.gene.2012.11.016.
Vara, J., Malpartida, F., Hopwood, D. A., and Jimenez, A. (1985). Cloning and expression of a puromycin N-acetyl transferase gene from Streptomyces alboniger in Streptomyces lividans and Escherichia coli. Gene 33, 197-206. 10.1016/0378-1119(85)90094-0.
Varshavsky, A. (2019). N-degron and C-degron pathways of protein degradation. Proc Natl Acad Sci USA 116, 358-366. 10.1073/pnas.1816596116.
Yates, J. L., Warren, N., and Sugden, B. (1985). Stable replication of plasmids derived from Epstein-Barr virus in various mammalian cells. Nature 313, 812-815. 10.1038/313812a0.
Zhang, F., Thornhill, S. I., Howe, S. J., Ulaganathan, M., Schambach, A., Sinclair, J., Kinnon, C., Gaspar, H. B., Antoniou, M., and Thrasher, A. J. (2007). Lentiviral vectors containing an enhancer-less ubiquitously acting chromatin opening element (UCOE) provide highly reproducible and stable transgene expression in hematopoietic cells. Blood 110, 1448-1457. 10.1182/blood-2006-12-060814.
Zuo, B., Qi, H., Lu, Z., Chen, L., Sun, B., Yang, R., Zhang, Y., Liu, Z., Gao, X., You, A., et al. (2020). Alarmin-painted exosomes elicit persistent antitumor immunity in large established tumors in mice. Nat Commun 11, 1790. 10.1038/s41467-020-15569-2.
R. Kalluri, V. S. LeBleu, The biology, function, and biomedical applications of exosomes. Science 367, (2020).
O. M. Elsharkasy, J. Z. Nordin, D. W. Hagey, O. G. de Jong, R. M. Schiffelers, S. E. Andaloussi, P. Vader, Extracellular vesicles as drug delivery systems: Why and how? Adv Drug Deliv Rev 159, 332-343 (2020).
S. J. Tsai, N. A. Atai, M. Cacciottolo, J. Nice, A. Salehi, C. Guo, A. Sedgwick, S. Kanagavelu, S. J. Gould, Exosome-mediated mRNA Delivery in vivo is safe and can be used to induce SARS-CoV-2 immunity. J Biol Chem, 101266 (2021).
K. W. Witwer, C. Thery, Extracellular vesicles or exosomes? On primacy, precision, and popularity influencing a choice of nomenclature. J Extracell Vesicles 8, 1648167 (2019).
A. Bobrie, M. Colombo, G. Raposo, C. Thery, Exosome secretion: molecular mechanisms and roles in immune responses. Traffic 12, 1659-1668 (2011).
C. Thery, M. Ostrowski, E. Segura, Membrane vesicles as conveyors of immune responses. Nat Rev Immunol 9, 581-593 (2009).
Y. Fang, N. Wu, X. Gan, W. Yan, J. C. Morrell, S. J. Gould, Higher-order oligomerization targets plasma membrane proteins and HIV gag to exosomes. PLoS Biol 5, e158 (2007).
B. Shen, Y. Fang, N. Wu, S. J. Gould, Biogenesis of the posterior pole is mediated by the exosome/microvesicle protein-sorting pathway. J Biol Chem 286, 44162-44176 (2011).
B. Shen, N. Wu, J. M. Yang, S. J. Gould, Protein targeting to exosomes/microvesicles by plasma membrane anchors. J Biol Chem 286, 14383-14395 (2011).
X. Gan, S. J. Gould, Identification of an inhibitory budding signal that blocks the release of HIV particles and exosome/microvesicle proteins. Mol Biol Cell 22, 817-830 (2011).
S. J. Tsai, Y. Ai, C. Guo, S. J. Gould, Degron-tagging of BleoR and other antibiotic-resistance genes selects for higher expression of linked transgenes and improved exosome engineering. J Biol Chem, 101846 (2022).
J. M. Yang, S. J. Gould, The cis-acting signals that target proteins to exosomes and microvesicles. Biochem Soc Trans 41, 277-282 (2013).
F. K. Fordjour, C. Guo, Y. Ai, G. G. Daaboul, S. J. Gould, A shared, stochastic pathway mediates exosome protein budding along plasma and endosome membranes. J Biol Chem, 102394 (2022).
F. K. Fordjour, G. G. Daaboul, S. J. Gould, A shared pathway of exosome biogenesis operates at plasma and endosome membranes. BiorXiv, 1-42 (2019).
A. M. Booth, Y. Fang, J. K. Fallon, J. M. Yang, J. E. Hildreth, S. J. Gould, Exosomes and HIV Gag bud from endosome-like domains of the T cell plasma membrane. J Cell Biol 172, 923-935 (2006).
C. Guo, S. J. Tsai, Y. Ai, M. Li, E. Anaya, A. Pekosz, A. Cox, S. J. Gould, The D614G mutation redirects SARS-CoV-2 spike to lysosomes and suppresses deleterious traits of the furin cleavage site insertion mutation. Sci Adv 8, eade5085 (2022).
J. Pallesen, N. Wang, K. S. Corbett, D. Wrapp, R. N. Kirchdoerfer, H. L. Turner, C. A. Cottrell, M. M. Becker, L. Wang, W. Shi, W. P. Kong, E. L. Andres, A. N. Kettenbach, M. R. Denison, J. D. Chappell, B. S. Graham, A. B. Ward, J. S. McLellan, Immunogenicity and structures of a rationally designed prefusion MERS-CoV spike antigen. Proc Natl Acad Sci USA 114, E7348-E7357 (2017).
D. Wrapp, N. Wang, K. S. Corbett, J. A. Goldsmith, C. L. Hsieh, O. Abiona, B. S. Graham, J. S. McLellan, Cryo-EM structure of the 2019-nCoV spike in the prefusion conformation. Science 367, 1260-1263 (2020).
D. R. Feikin, M. M. Higdon, L. J. Abu-Raddad, N. Andrews, R. Araos, Y. Goldberg, M. J. Groome, A. Huppert, K. L. O'Brien, P. G. Smith, A. Wilder-Smith, S. Zeger, M. Deloria Knoll, M. K. Patel, Duration of effectiveness of vaccines against SARS-CoV-2 infection and COVID-19 disease: results of a systematic review and meta-regression. Lancet 399, 924-944 (2022).
C. E. McBride, J. Li, C. E. Machamer, The cytoplasmic tail of the severe acute respiratory syndrome coronavirus spike protein contains a novel endoplasmic reticulum retrieval signal that binds COPI and promotes interaction with membrane protein. J Virol 81, 2418-2428 (2007).
B. C. Jennings, S. Kornfeld, B. Doray, A weak COPI binding motif in the cytoplasmic tail of SARS-CoV-2 spike glycoprotein is necessary for its cleavage, glycosylation, and localization. FEBS Lett, (2021).
C. Barlowe, A. Helenius, Cargo Capture and Bulk Flow in the Early Secretory Pathway. Annu Rev Cell Dev Biol 32, 197-222 (2016).
A. V. Weigel, C. L. Chang, G. Shtengel, C. S. Xu, D. P. Hoffman, M. Freeman, N. Iyer, J. Aaron, S. Khuon, J. Bogovic, W. Qiu, H. F. Hess, J. Lippincott-Schwartz, ER-to-Golgi protein delivery through an interwoven, tubular network extending from ER. Cell 184, 2412-2429 e2416 (2021).
C. S. Sevier, O. A. Weisz, M. Davis, C. E. Machamer, Efficient export of the vesicular stomatitis virus G protein from the endoplasmic reticulum requires a signal in the cytoplasmic tail that includes both tyrosine-based and di-acidic motifs. Mol Biol Cell 11, 13-22 (2000).
E. A. Miller, T. H. Beilharz, P. N. Malkus, M. C. Lee, S. Hamamoto, L. Orci, R. Schekman, Multiple cargo binding sites on the COPII subunit Sec24p ensure capture of diverse membrane proteins into transport vesicles. Cell 114, 497-509 (2003).
J. D. Mancias, J. Goldberg, Structural basis of cargo membrane protein discrimination by the human COPII coat machinery. EMBO J 27, 2918-2928 (2008).
M. B. Zwick, R. Jensen, S. Church, M. Wang, G. Stiegler, R. Kunert, H. Katinger, D. R. Burton, Anti-human immunodeficiency virus type 1 (HIV-1) antibodies 2F5 and 4E10 require surprisingly few crucial residues in the membrane-proximal external region of glycoprotein gp41 to neutralize HIV-1. J Virol 79, 1252-1261 (2005).
D. J. Salamango, K. K. Alam, D. H. Burke, M. C. Johnson, In Vivo Analysis of Infectivity, Fusogenicity, and Incorporation of a Mutagenic Viral Glycoprotein Library Reveals Determinants for Virus Incorporation. J Virol 90, 6502-6514 (2016).
J. K. Rose, W. J. Welch, B. M. Sefton, F. S. Esch, N. C. Ling, Vesicular stomatitis virus glycoprotein is anchored in the viral membrane by a hydrophobic domain near the COOH terminus. Proc Natl Acad Sci USA 77, 3884-3888 (1980).
M. Feary, A. J. Racher, R. J. Young, C. M. Smales, Methionine sulfoximine supplementation enhances productivity in GS-CHOKISV cell lines through glutathione biosynthesis. Biotechnol Prog 33, 17-25 (2017).
L. E. Quek, S. Dietmair, J. O. Kromer, L. K. Nielsen, Metabolic flux analysis in mammalian cell culture. Metab Eng 12, 161-171 (2010).
K. Dooley, R. E. McConnell, K. Xu, N. D. Lewis, S. Haupt, M. R. Youniss, S. Martin, C. L. Sia, C. McCoy, R. J. Moniz, O. Burenkova, J. Sanchez-Salazar, S. C. Jang, B. Choi, R. A. Harrison, D. Houde, D. Burzyn, C. Leng, K. Kirwin, N. L. Ross, J. D. Finn, L. Gaidukov, K. D. Economides, S. Estes, J. E. Thornton, J. D. Kulman, S. Sathyanarayanan, D. E. Williams, A versatile platform for generating engineered extracellular vesicles with defined therapeutic properties. Mol Ther 29, 1729-1743 (2021).
S. J. Gould, A. M. Booth, J. E. Hildreth, The Trojan exosome hypothesis. Proc Natl Acad Sci USA 100, 10592-10597 (2003).
S. J. Gould, G. Raposo, As we wait: coping with an imperfect nomenclature for extracellular vesicles. J Extracell Vesicles 2, (2013).
X. Wu, T. A. Rapoport, Mechanistic insights into ER-associated protein degradation. Curr Opin Cell Biol 53, 22-28 (2018).
Z. Sun, J. L. Brodsky, Protein quality control in the secretory pathway. J Cell Biol 218, 3171-3187 (2019).
B. H. Sung, T. Ketova, D. Hoshino, A. Zijlstra, A. M. Weaver, Directional cell movement through tissues is controlled by exosome secretion. Nat Commun 6, 7164 (2015).
W. Zheng, R. He, X. Liang, S. Roudi, J. Bost, P. M. Coly, G. V. Niel, S. E. L. Andaloussi, Cell-specific targeting of extracellular vesicles though engineering the glycocalyx. J Extracell Vesicles 11, e12290 (2022).
S. Kol, D. Ley, T. Wulff, M. Decker, J. Arnsdorf, S. Schoffelen, A. H. Hansen, T. L. Jensen, J. M. Gutierrez, A. W. T. Chiang, H. O. Masson, B. O. Palsson, B. G. Voldborg, L. E. Pedersen, H. F. Kildegaard, G. M. Lee, N. E. Lewis, Multiplex secretome engineering enhances recombinant protein production and purity. Nat Commun 11, 1908 (2020).

TABLE 2
DNA sequences

| Sequence | |
|---|---|
| ID | |
| Number | Sequence |
| SEQ ID NO: 1 | Cas9/gRNA target site |
| | gggattaatt tcaacgagaa ggcgatccat |
| | aaggaggtag ggaggagact gt |
| SEQ ID NO: 2 | Allele 1 in the F/CD63-/- cell line |
| | gggattaatt tcaacgagaa |
| | ggcgtaggga ggagactgt |
| SEQ ID NO: 3 | Allele 2 in the F/CD63-/- cell line |
| | gggattaatt tcaacgagaa |
| | ggcgatccta gggaggagac tgt |
| SEQ ID NO: 4 | Primer 1 for CD63 |
| | cagtggtcat catcgcagtg |
| SEQ ID NO: 5 | Primer 2 for CD63 |
| | atcgaagcag tgtggttgtt t |
| SEQ ID NO: 6 | Primer 1 for 18S rRNA |
| | cggcgacgac ccattcgaac |
| SEQ ID NO: 7 | Primer 2 for 18S rRNA |
| | gaatcgaacc ctgattcccc gtc |

TABLE 3
Amino acid sequences

| Sequence | |
|---|---|
| ID | |
| Number | Sequence |
| SEQ ID NO: 8 | CP05 peptide |
| | CRHSQMTVTS RL |
| SEQ ID NO: 9 | T1EMAv1 |
| | DLESSQGWFE GLFNRSPWFT TKALFYFVFF |
| | YPFPIFGILI ITILLVRLQK |
| | RRERRRTEDL EMGRIPRRA |
| SEQ ID NO: 10 | T1EMAv2 |
| | DLESSQGWFE GLFNRSPWFT TTLFVPLLVG |
| | TGVALVTGAT VLGTITCCFM |
| | RLQKRRERRR TEDLEMGRIP RRA |

### ILLUSTRATIVE EMBODIMENTS

1. An isolated polynucleotide sequence encoding a polypeptide comprising a selectable marker (SM) protein, a degron domain (DD) and an exosome cargo (EC) protein in operable linkage.
2. The isolated polynucleotide of claim 1, wherein the polynucleotide encodes a fusion protein.
3. The isolated polynucleotide of claim 2, comprising from a 5′ end to a 3′ end, an EC protein, a DD, and an SM protein.
4. The isolated polynucleotide of claim 3, further comprising a first linker between the EC and the DD, and a second linker between the DD and the SM.
5. The isolated polynucleotide of claim 4, wherein the second linker is cleavable or self-cleavable.
6. The isolated polynucleotide of any of claims 1-5, wherein the SM protein is zeocin resistance protein (BleoR), blasticidin resistance protein (BsdR), G418 resistance protein (NeoR), puromycin resistance protein (PuroR), hygromycin resistance protein (HygR), or a combination thereof.
7. The isolated polynucleotide of claim 6, wherein the SM protein is BleoR.
8. The isolated polynucleotide of claim 6, wherein the SM protein is PuroR.
9. The isolated polynucleotide of any of claims 1-5, wherein the DD is ER50 derived from a human estrogen receptor, or ecDHFR derived from E. coli DHFR.
10. The isolated polynucleotide of claim 4, wherein the second linker is a self-cleavable viral 2a peptide.
11. The isolated polynucleotide of any of claims 1-8, wherein one or more of the coding sequences are operably linked to a regulatory control element.
12. The isolated polynucleotide of claim 11, wherein the regulatory control element comprises a CMV promoter.
13. The isolated polynucleotide of any of claims 1-12, wherein expression of one or more of the coding sequences in a cell increases the amount of exosomes produced by the cell.
14. The isolated polynucleotide of any of claims 1-12, wherein expression of one or more of the coding sequences in a cell increases the amount of EC protein within exosomes produced by the cell.
15. The isolated polynucleotide of claim 14, wherein the amount of exosomes produced by the cell increases by about at least 500% as compared to a cell that does not comprise the isolated polynucleotide.
16. The isolated polynucleotide of claim 14, wherein the amount of EC protein within the exosomes is at least about 20-fold higher than an amount of EC protein in exosomes produced by a cell comprising the isolated polynucleotide lacking a sequence encoding a DD.
17. An isolated polynucleotide comprising a sequence encoding a modified antigen, wherein the sequence encoding the modified antigen comprises the isolated polynucleotide of any of claims 1-16.
18. An isolated cell comprising the polynucleotide of any of claims 1-17.
19. A method of producing exosomes comprising:
   a) introducing an isolated polynucleotide of any of claims 1-17 into a cell in a first culture media;
   b) contacting the cells of a) with an antibiotic in a second culture media comprising the antibiotic, thereby selecting antibiotic resistant cells;
   c) optionally contacting the cells of a) with culture media that does not comprise a compound for cell growth;
   c) expanding the antibiotic resistant cells of b) in a third culture media;
   d) culturing the expanded antibiotic resistant cells of c) in a fourth culture media; and
   e) harvesting exosomes from the fourth culture media, thereby producing exosomes.
20. The method of claim 19, wherein the antibiotic is zeocin.
21. The method of claim 19, wherein the antibiotic is puromycin.
22. The method of claim 19, wherein the compound essential for cell growth is glutamine.
23. A pharmaceutical composition comprising an exosome produced by the method of any of claims 19-22.
24. A method for producing extracellular vesicle (EV) in a culture media comprising: (i) inserting an isolated polynucleotide encoding a coding region for an exosome cargo protein (EC) into an expression vector configured to drive recombinant EC expression; (ii) transfecting the expression vector into a cell suitable for producing EVs, thereby generating a transgenic cell; (iii) contacting the transgenic cells with an antibiotic, thereby producing a transgenic cell that expresses a high level of the recombinant EC; (iv) expanding the cell of (iii) in culture media to produce a conditioned culture media; and (iv) collecting EVs from the conditioned culture media.
25. The method of claim 24, wherein the coding region comprises from a 5′ to a 3′ end:
   a) a first inverted tandem repeat (ITR-1) flanking
   b) a region comprising: a promoter, an exosome cargo protein (EC), a linker peptide (LP), an antibiotic resistance protein (AR), a woodchuck hepatitis virus post-transcriptional regulatory element (WPRE) and a polyadenylation site (pAn);
   c) a second ITR (ITR-r);
   d) a Rous sarcoma virus long-terminal repeat (RSV);
   e) a Sleeping Beauty transposase SB100x; and
   f) a polyadenylation site (pAn).
26. The method of claim 25, wherein the AR is selected from the group consisting of zeocin resistance protein (BleoR), blasticidin resistance protein (BsdR), G418 resistance protein (NeoR), puromycin resistance protein (PuroR), hygromycin resistance protein (HygR), and a combination thereof.
27. The method of claim 26, wherein the AR is linked to a degron domain (DD).
28. The method of claim 27, wherein the DD is ER50 derived from the human estrogen receptor, or ecDHFR derived from E. coli DHFR.
29. The method of claim 25, wherein the EC is CD63/Y235A.
30. The method of claim 29, wherein high-level expression of CD63/Y235A leads to about 5-fold increase in EV production yield.
31. A method for producing an extracellular vesicle (EV) in a culture media comprising: (i) inserting an isolated polynucleotide encoding a coding region for an exosome cargo protein (EC) into an expression vector configured to drive recombinant EC expression; (ii) transfecting the expression vector into a cell line suitable for producing EVs, thereby generating a transgenic cell; (iii) contacting the transgenic cell with an antibiotic, thereby producing a transgenic cell that expresses a high level of the recombinant EC; (iv) contacting the transgenic cell with a culture media that does not comprise an essential compound for cell growth; (v) expanding the cell of (iv) in culture media to produce a conditioned culture media; and (vi) collecting EVs from the conditioned culture media.
32. The method of claim 31, wherein the coding region comprises from a 5′ to a 3′ end:
   a) a first inverted tandem repeat (ITR-1) flanking
   b) a region comprising: a selectable marker system (SMS), a promoter, an exosome cargo protein (EC), a woodchuck hepatitis virus post-transcriptional regulatory element (WPRE) and a polyadenylation site (pAn);
   c) a second ITR (ITR-r);
   d) a Rous sarcoma virus long-terminal repeat (RSV);
   e) a Sleeping Beauty transposase SB100x; and
   f) a polyadenylation site (pAn).
33. The method of claim 32, wherein the SMS encodes a polypeptide comprising a glutamine synthase (GS) protein, a porcine teschovirus 2a peptide linker and an antibiotic resistance (AR) protein.
34. The method of claim 32, wherein the SMS further comprises a promoter.
35. The method of claim 32, wherein the EC comprises a modified antigen.
36. The method of claim 31, wherein the EVs are exosomes or microvesicles.
37. The method of claim 31, wherein the cell suitable for producing EVs is a 293F-derived cell.
38. The method of claim 35, wherein about 50% of the EVs comprise a modified SARS-CoV-2 Spike protein.
39. A pharmaceutical composition comprising an exosome produced by the method of any of claims 31-38.
40. An expression vector wherein the coding region comprises from a 5′ to a 3′ end:
   a) a first inverted tandem repeat (ITR-1) flanking
   b) a region comprising: a promoter, an exosome cargo protein (EC), a linker peptide (LP), an antibiotic resistance protein (AR), a woodchuck hepatitis virus post-transcriptional regulatory element (WPRE) and a polyadenylation site (pAn);
   c) a second ITR (ITR-r)
   d) a Rous sarcoma virus long-terminal repeat (RSV);
   e) a Sleeping Beauty transposase SB100x; and
   f) a polyadenylation site (pAn).
41. The expression vector of claim 40, wherein the EC is CD63/Y235A.
42. The expression vector of claim 40, wherein the EC comprises a modified antigen.
43. The expression vector of claim 40, wherein the AR is selected from the group consisting of zeocin resistance protein (BleoR), blasticidin resistance protein (BsdR), G418 resistance protein (NeoR), puromycin resistance protein (PuroR), hygromycin resistance protein (HygR), and a combination thereof.
44. The expression vector of claim 40, wherein the AR is linked to a degron domain (DD).
45. The expression vector of claim 44, wherein the DD is ER50 derived from a human estrogen receptor, or ecDHFR derived from E. coli DHFR.
46. An isolated polynucleotide sequence encoding a modified antigen, wherein the modified antigen comprises a modified lysosome sorting peptide, a linker protein, and a modified COPI-binding CTT peptide.
47. The isolated polynucleotide of claim 46, wherein the modified lysosome sorting peptide disrupts delivery of the modified antigen to lysosomes and increases delivery of the modified antigen to a plasma membrane.
48. The isolated polynucleotide of claim 46, wherein the modified antigen has increased immunogenicity compared to a naturally occurring antigen.
49. The isolated polynucleotide of claim 46, wherein the linker protein is the porcine teschovirus 2a peptide.
50. The isolated polynucleotide of claim 46, wherein the modified antigen is a SARS-CoV-2 Spike protein.
51. The modified SARS-CoV-2 Spike protein of claim 50 wherein the modified lysosome sorting peptide comprises a diproline substitution.
52. The modified SARS-CoV-2 Spike protein of claim 51, wherein the diproline substitution comprises 986KV987-to-986PP987.
53. The modified SARS-CoV-2 Spike protein of claim 52, wherein the diproline substitution increases cell surface Spike protein expression by about 500%.
54. The modified SARS-CoV-2 Spike protein of claim 50, wherein the modified COPI-binding CTT peptide comprises a diacidic ER export signal (ERES).
55. The modified SARS-CoV-2 Spike protein of claim 50, wherein the SARS-CoV-2 Spike protein is a SARS-CoV-2 Spike protein from a SARS-CoV-2 delta (S^{delta}) virus.
56. The modified SARS-CoV-2 Spike protein of claim 55, wherein the modified SARS-CoV-2 Spike protein comprises a mutation selected from the group consisting of T19R, G142D, D157-158, L452R, T478K, D614G, P681R, and D950N.
57. The modified SARS-CoV-2 Spike protein of claim 50, wherein the modified SARS-CoV-2 Spike protein further comprises a furin cleavage site mutation.
58. The modified SARS-CoV-2 Spike protein of claim 57, wherein the furin cleavage site eliminates biogenic processing of full-length Spike into S1 and S2 components.
59. The modified SARS-CoV-2 Spike protein of claim 57, wherein the furin cleavage site mutation comprises 682RRAR685-to-682GSAG685.
60. The modified SARS-CoV-2 Spike protein of claims 50-59, wherein the modified SARS-CoV-2 Spike protein comprises mutations present in a virulent strain of the SARS-CoV-2 virus (S^{delta}) a furin cleavage site mutation (CSM), diproline substitutions (2P), and a deleted COPI-binding CTT peptide replaced with a CTT peptide carrying a diacidic ER export signal (ΔC-ERES).
61. The modified SARS-CoV-2 Spike protein of claim 60, wherein the modified SARS-CoV-2 spike protein is expressed on a cell surface about 500% more than an unmodified SARS-CoV-2 Spike protein.
62. An isolated polynucleotide comprising a sequence encoding a metabolic selectable marker (MSM), a linker peptide (LP), and an antibiotic resistance protein (AR).
63. The isolated polynucleotide of claim 62, wherein the MSM is a doxycycline-regulated Tet-on sequence rtTAv16 transcription factor or a glutamine synthetase.
64. The isolated polynucleotide of claim 62, wherein the MSM is glutamine synthetase.
65. The isolated polynucleotide of claim 62, wherein the LP is a viral p2a peptide.
66. The isolated polynucleotide of claim 62, wherein the LP is a porcine teschovirus 2a peptide.
67. The isolated polynucleotide of claim 62, wherein the AR is selected from the group consisting of BleoR, PuroR, PuroR2, BsdR, NeoR, and HygR.
68. The isolated polynucleotide of claim 72, wherein the AR is BleoR, PuroR, or PuroR2.
69. An isolated polynucleotide sequence encoding a modified antigen comprising a non-Spike membrane-proximal external region (MPER), a transmembrane domain (TMD) and a carboxy-terminal tail (CTT).
70. The isolated polynucleotide of claim 69, wherein the MPER is a murine leukemia virus envelope glycoprotein (MLV MPER), a membrane-proximal external region of the human immunodeficiency virus type 1 (HIV MPER), or a vesicular stomatitis virus glycoprotein (VSVG).
71. The isolated polynucleotide of claim 69, wherein the MPER is MLV MPER.
72. The isolated polynucleotide of claim 69, wherein the TMD is a type-1 exosomal membrane protein from an immunoglobulin superfamily.
73. The isolated polynucleotide of claim 69, wherein the TMD is IgSF2, IgSF3, or IgSF8.
74. The isolated polynucleotide of claim 69, wherein the TMD is IgSF3 type-1 exosomal membrane protein (IgSF3 TMD).
75. The isolated polynucleotide of claim 69, wherein the CTT comprises a diacidic ER export signal (ERES) and a Carajas virus G protein (CTT5).
76. The isolated polynucleotide of claim 69, wherein the CTT comprises an ERES and a fusion of CTT5 with a Golgi export signal of reovirus p14 (CTT6).
77. The isolated polynucleotide of claim 69, wherein the MPER is MLV, the TMD is IgSF3, and the CTT is CTT6.
78. The isolated polynucleotide of claim 69, wherein the MPER is MLV, the TMD is IgFS8, and the CTT is CTT6.
79. The isolated polynucleotide of claim 69, wherein the modified antigen is a SARS-CoV-2 Spike protein.
80. The isolated polynucleotide of claim 79, wherein the SARS-CoV-2 Spike protein is a SARS-Cov2-Spike protein from a SARS-CoV-2 delta (S^{delta}) virus.
81. The isolated polynucleotide of claim 69, wherein the modified antigen is an extracellular domain of influenza hemagglutinin (HA).
82. The isolated polynucleotide of claim 69, wherein the modified antigen is an extracellular domain of vascular endothelial growth factor (VEGFR) and a constant region of the human immunoglobulin heavy chain (IgG Fc).
83. The isolated polynucleotide of claim 69, wherein the modified antigen is a modified alpha galactosidase A (GLA).
84. An exosome-based vaccine, wherein the vaccine comprises an exosome comprising the modified antigen of any of claims 69-83.
85. An expression vector wherein the coding region comprises from a 5′ to a 3′ end:
   a) a first inverted tandem repeat (ITR-1) flanking
   b) a region comprising: a selectable marker system (SMS), a promoter, an exosome cargo protein (EC), a woodchuck hepatitis virus post-transcriptional regulatory element (WPRE) and a polyadenylation site (pAn);
   c) a second ITR (ITR-r)
   d) a Rous sarcoma virus long-terminal repeat (RSV);
   e) a Sleeping Beauty transposase SB100x; and
   f) a polyadenylation site (pAn).
86. The expression vector of claim 85, wherein the SMS encodes a polypeptide comprising a glutamine synthase (GS) protein, a porcine teschovirus 2a peptide linker and an antibiotic resistance (AR) protein.
87. The expression vector of claim 86, wherein the SMS further comprises a promoter.
88. The expression vector of claim 85, wherein the EC is a modified SARS-CoV-2 Spike protein.
89. The expression vector of claim 85, wherein the EC is a modified alpha galactosidase A (GLA).
90. The expression vector of claim 85, wherein the EC is an extracellular domain of vascular endothelial growth factor (VEGFR) and a constant region of a human immunoglobulin heavy chain (IgG Fc).
91. The expression vector of claim 85, wherein the EC is a heavy chain of trastuzumab and a light chain of trastuzumab.
92. A method for producing extracellular vesicles (“EVs”), comprising the steps of: (i) inserting the coding region for an exosome carrier protein (“ECP”) into an expression vector that is configured to drive the recombinant ECP expression; (ii) transfecting the expression vector into a cell line suitable for producing EVs; (iii) selecting and growing a transgenic cell line that expresses a high level of the recombinant ECP in culture media; and (iv) collecting EVs from the conditioned tissue culture media.
93. The method of claim 92, wherein the transgenic cell line that expresses a high level of the recombinant ECP in the step (iii) is a transgenic cell line that expresses the highest level of the recombinant ECP.
94. The method of claim 92-93, wherein the ECP is CD63/Y235A, CD9, or TSPAN7.
95. The method of claim 94, wherein the high-level expression of CD63/Y235A leads to approximately 5-fold increase in the EV production yield.
96. The method of claim 94, wherein the high-level expression of CD9 leads to approximately 10-fold increase in the EV production yield.
97. The method of claim 94, wherein the high-level expression of TSPAN7 leads to approximately 20-fold increase in the EV production yield.
98. The method of any of the preceding claims, wherein the EVs are exosomes or microvesicles.
99. The method of any of the preceding claims, wherein the cell line suitable for producing EVs is a 293F-derived cell.
100. An expression vector for producing EVs, comprising the coding region for an ECP.
101. The expression vector of claim 100, wherein the ECP is CD63/Y235A, CD9, or TSPAN7.
102. The expression vector of claims 100-101, wherein the EVs are exosomes or microvesicles.
103. A cell line for producing EVs, comprising the expression vector of claims 100-102.
104. The cell line of claim 103, wherein the EVs are exosomes or microvesicles.

Although the invention has been described with reference to the presently preferred embodiments, it should be understood that various modifications can be made without departing from the spirit of the invention. Accordingly, the invention is limited only by the following claims.

1. An isolated polynucleotide sequence encoding a polypeptide comprising from 5′ to 3′, an exosome cargo (EC) protein, a degron domain (DD), and a selectable marker (SM) protein in operable linkage, wherein the polypeptide is a modified antibiotic resistance (AR) protein.

2. The isolated polynucleotide of claim 1, further comprising a first linker between the EC and the DD, and a second linker between the DD and the SM, wherein the second linker is cleavable or self-cleavable.

3. The isolated polynucleotide of claim 1, wherein the SM protein is zeocin resistance protein (BleoR), blasticidin resistance protein (BsdR), G418 resistance protein (NeoR), puromycin resistance protein (PuroR), hygromycin resistance protein (HygR), a protein homologous to PuroR (PuroR2), a protein homologous to HygR, or a combination thereof.

4. The isolated polynucleotide of claim 1, wherein the DD is ER50 derived from a human estrogen receptor, or ecDHFR derived from E. coli DHFR.

5. The isolated polynucleotide of claim 1, wherein expression of one or more of the coding sequences in a cell increases the amount of exosomes produced by the cell.

6. An isolated polynucleotide sequence encoding a modified antigen comprising a type-1 exosome membrane anchor (T1EMA) peptide, wherein the T1EMA comprises a membrane-proximal external region (MPER), a transmembrane domain (TMD) and a carboxy-terminal tail (CTT).

7. The isolated polynucleotide of claim 6, wherein the MPER is a murine leukemia virus envelope glycoprotein (MLV MPER), a membrane-proximal external region of the human immunodeficiency virus type 1 (HIV MPER), or a vesicular stomatitis virus glycoprotein (VSVG).

8. The isolated polynucleotide of claim 6, wherein the TMD is a type-1 exosomal membrane protein from an immunoglobulin superfamily.

9. The isolated polynucleotide of claim 6, wherein the CTT comprises a diacidic ER export signal (ERES) and a Carajas virus G protein (CTT5).

10. The isolated polynucleotide of claim 6, wherein the CTT comprises an ERES and a fusion of CTT5 with a Golgi export signal of reovirus p14 (CTT6).

11. The isolated polynucleotide of claim 6, wherein the MPER is MLV, the TMD is IgSF3, and the CTT is CTT6.

12. The isolated polynucleotide of claim 6, wherein the MPER is MLV, the TMD is IgFS8, and the CTT is CTT6.

13. The isolated polynucleotide of claim 6, wherein the modified antigen comprises a SARS-CoV-2 Spike protein.

14. The isolated polynucleotide of claim 13, wherein the SARS-CoV-2 Spike protein is a SARS-Cov2-Spike protein from a SARS-CoV-2 delta (S^{delta}) virus.

15. The isolated polynucleotide of claim 6, wherein the modified antigen comprises an extracellular domain of influenza hemagglutinin (HA).

16. The isolated polynucleotide sequence of claim 6, wherein expression of the isolated polynucleotide sequence increases the amount of exosomes produced by a cell.

17. An isolated polynucleotide sequence encoding a polypeptide comprising a TSPAN7/Y246A, wherein expression of the isolated polynucleotide sequence increases the amount of exosomes produced by a cell.

18. The polypeptide of claim 17, further comprising a heterologous peptide.

19. A method of increasing the number of exosomes produced by a cell comprising:
a) introducing an isolated polynucleotide of claim 18 into a cell in a first culture media;
b) culturing the cells of a) in a second culture media; and
c) harvesting exosomes from the third culture media, wherein the number of exosomes produced is increased relative to a cell not including the polynucleotide of a),
thereby increasing the number of exosomes produced by the cell.

20. A method of delivering a heterologous peptide to an exosome comprising introducing the isolated polynucleotide of claim 18 into a cell and culturing the cell in a culture media, thereby delivering the heterologous peptide to an exosome.

21.-33. (canceled)


SEQUENCE LISTING (10 sequences)

SEQ ID NO: 1 - DNA, 52 bp, synthetic construct
  misc_feature 1..52: note = Cas9/gRNA target site
  misc_feature 13..30: note = gRNATarget
  source 1..52: mol_type = other DNA
gggattaatttcaacgagaaggcgatccataaggaggtagggaggagactgt

SEQ ID NO: 2 - DNA, 39 bp, synthetic construct
  misc_feature 1..39: note = Allele 1 in the F/CD63-/- cell line
  source 1..39: mol_type = other DNA
gggattaatttcaacgagaaggcgtagggaggagactgt

SEQ ID NO: 3 - DNA, 43 bp, synthetic construct
  misc_feature 1..43: note = Allele 2 in the F/CD63-/- cell line
  source 1..43: mol_type = other DNA
gggattaatttcaacgagaaggcgatcctagggaggagactgt

SEQ ID NO: 4 - DNA, 20 bp, synthetic construct
  misc_feature 1..20: note = primer for CD63
  source 1..20: mol_type = other DNA
cagtggtcatcatcgcagtg

SEQ ID NO: 5 - DNA, 21 bp, synthetic construct
  misc_feature 1..21: note = Primer for CD63
  source 1..21: mol_type = other DNA
atcgaagcagtgtggttgttt

SEQ ID NO: 6 - DNA, 20 bp, synthetic construct
  misc_feature 1..20: note = primer for 18S rRNA
  source 1..20: mol_type = other DNA
cggcgacgacccattcgaac

SEQ ID NO: 7 - DNA, 23 bp, synthetic construct
  misc_feature 1..23: note = Primer for 18S rRNA
  source 1..23: mol_type = other DNA
gaatcgaaccctgattccccgtc

SEQ ID NO: 8 - protein, 12 aa, synthetic construct
  REGION 1..12: note = CP05 peptide
CRHSQMTVTSRL

SEQ ID NO: 9 - protein, 69 aa, synthetic construct
  REGION 1..69: note = T1EMAv1
DLESSQGWFEGLFNRSPWFTTKALFYFVFFYPFPIFGILIITILLVRLQKRRERRRTEDLEMGRIPRRA

SEQ ID NO: 10 - protein, 73 aa, synthetic construct
  REGION 1..73: note = T1EMAv2
DLESSQGWFEGLFNRSPWFTTTLFVPLLVGTGVALVTGATVLGTITCCFMRLQKRRERRRTEDLEMGRIPRRA